(19) United States
(12) Patent Application Publication
(10) Pub. No.: US 2023/0088452 A1
(43) Pub. Date: Mar. 23, 2023

(54) METHODS FOR DETECTING NTRK GENE FUSION USING RNA IN SITU HYBRIDIZATION

(71) Applicants: ADVANCED CELL DIAGNOSTICS, INC., Newark, CA (US); THE GENERAL HOSPITAL CORPORATION, Boston, MA (US)

(72) Inventors: Xiao-Jun Ma, Newark, CA (US); Robert J. Monroe, Newark, CA (US); David T. Ting, Boston, MA (US); Miguel N. Rivera, Boston, MA (US); Vikram Deshpande, Boston, MA (US); Azfar Neyaz, Boston, MA (US); Baris Boyraz, Boston, MA (US)

(21) Appl. No.: 17/904,568

(22) PCT Filed: Feb. 24, 2021
(86) PCT No.: PCT/US2021/019426

§ 371 Date: Aug. 18, 2022

Related U.S. Application Data
(60) Provisional application No. 62/983,513, filed on Feb. 28, 2020.

(51) Int. Cl. C12Q 1/6886

(52) U.S. Cl. CPC C12Q 1/6886; C12Q 2600/156

(57) ABSTRACT

A method of detecting a neurotrophic tyrosine receptor kinase (NTRK) fusion gene, comprising obtaining a sample and detecting a NTRK fusion gene in the sample by RNA in situ hybridization using a probe comprising a nucleic acid sequence complementary to a region of mRNA encoding a kinase domain of NTRK1, NTRK2, or NTRK3.

### 1. RELATED APPLICATIONS

This application claims priority to and the benefit of U.S. Provisional Patent Application No. 62/983,513 filed Feb. 28, 2020, which is incorporated herein by reference in its entirety for all purposes.

### 2. INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 69,084 Byte ASCII (Text) file named “2021-02-23_38638-601_SQL ST25.txt,” created on Feb. 23, 2021.

### 3. FIELD

The present disclosure relates generally to detection of neurotrophic tyrosine kinase (NTRK) gene fusion.

### 4. BACKGROUND

Cancer is characterized primarily by an increase in the number of abnormal cells derived from a given normal tissue, invasion of adjacent tissues by these abnormal cells, or lymphatic or blood-borne spread of malignant cells to regional lymph nodes and to distant sites (metastasis). Cancers can be categorized by where in the body it started or the type of tissue from which it developed, such as breast cancer or lung cancer. Cancers can also be categorized by shared molecular alterations, regardless of the location of the cancers. In other words, the common denominator of the cancers with different tumor sites or histology in the same group is the presence of certain biomarkers for the shared molecular alterations. Accordingly, several tissue-agnostic treatments that target the specific molecular alterations have been developed for the corresponding group of cancers. The accompanying diagnostic testing for the molecular alterations, therefore, become essential in the clinicians' decision in proper administration of the tissue-agnostic treatments.

Different technologies have the potential to be employed in the detection of certain specific molecular alterations on levels of DNA, RNA, or protein. Each technology differs in sensitivity, specificity, multiplexity, turnaround time, requirement for special facilities, tissue-efficiency, cost-efficiency. Considering the different profiles of the above attributes for a certain technology and in the context of prevalence of the related cancers, one could be best suited for a screening tool, and the other represents a better candidate for a confirmation method. For certain molecular alterations which are scattered across different cancers and only enriched in certain cancers, it can be challenging to detect them. Thus, there exists a need for optimizing detection methods for specific molecular alterations. The present disclosure satisfies this need and provides related advantages as well.

### 5. SUMMARY

In one aspect, provided herein is a method of detecting a neurotrophic tyrosine receptor kinase (NTRK) fusion gene, comprising: (i) obtaining a sample; and (ii) detecting a NTRK fusion gene in the sample by RNA in situ hybridization using a probe comprising a nucleic acid sequence complementary to a region of mRNA encoding a kinase domain of NTRK1, NTRK2, or NTRK3.

In some embodiments, the probe comprises a nucleic acid sequence complementary to a region of the mRNA encoding the kinase domain of NTRK1. In one embodiment, the probe comprises a nucleic acid sequence complementary to a region within SEQ ID NO:3, SEQ ID NO:5, SEQ ID NO:6, or SEQ ID NO:7.

In some embodiments, the probe comprises a nucleic acid sequence complementary to a region of the mRNA encoding the kinase domain of NTRK2. In one embodiment, the probe comprises a nucleic acid sequence complementary to a region within SEQ ID NO:10, SEQ ID NO:12, SEQ ID NO:13 or SEQ ID NO:14.

In some embodiments, the probe comprises a nucleic acid sequence complementary to a region of the mRNA encoding the kinase domain of NTRK3. In one embodiment, the probe comprises a nucleic acid sequence complementary to a region within SEQ ID NO:17, SEQ ID NO:19, SEQ ID NO:20 or SEQ ID NO:21.

In some embodiments, the method comprises detecting a NTRK fusion gene in the sample by RNA in situ hybridization using a probe pool comprising at least two of (a) a first probe comprising a nucleic acid sequence complementary to a region of the mRNA encoding the kinase domain of NTRK1; (b) a second probe comprising a nucleic acid sequence complementary to a region of the mRNA encoding the kinase domain of NTRK2; and (c) a third probe comprising a nucleic acid sequence complementary to a region of the mRNA encoding the kinase domain of NTRK3. In one embodiment, the probe pool comprises the first probe comprising a nucleic acid sequence complementary to a region of the mRNA encoding the kinase domain of NTRK1; and the second probe comprising a nucleic acid sequence complementary to a region of the mRNA encoding the kinase domain of NTRK2. In another embodiment, the probe pool comprises the first probe comprising a nucleic acid sequence complementary to a region of the mRNA encoding the kinase domain of NTRK1; and the third probe comprising a nucleic acid sequence complementary to a region of the mRNA encoding the kinase domain of NTRK3. In yet another embodiment, the probe pool comprises the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3. In still another embodiment, the probe pool comprises the first probe comprising a nucleic acid sequence complementary to a region of the mRNA encoding the kinase domain of NTRK1; the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3. In some embodiments, the first probe comprises a nucleic acid sequence complementary to a region within SEQ ID NO:3, SEQ ID NO:5, SEQ ID NO:6, or SEQ ID NO:7, the second probe comprises a nucleic acid sequence complementary to a region within SEQ ID NO:10, SEQ ID NO:12, SEQ ID NO:13, or SEQ ID NO:14, and the third probe comprises a nucleic acid sequence complementary to a region within SEQ ID NO:17, SEQ ID NO:19, SEQ ID NO:20, or SEQ ID NO:21.

In some embodiments, the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, SQSTM1-NTRK2, ETV6-NTRK3, BTBD1-NTRK3, EML4-NTRK3, TFG-NTRK3, RBPMS-NTRK3, LYN-NTRK3, and NTRK3-HOMER2. In specific embodiments, the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, and MRPL24-NTRK1. In other specific embodiments, the NTRK fusion is selected from a group consisting of OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, and SQSTM1-NTRK2. In yet other specific embodiments, the NTRK fusion is selected from a group consisting of ETV6-NTRK3, BTBD1-NTRK3, EML4-NTRK3, TFG-NTRK3, RBPMS-NTRK3, LYN-NTRK3, and NTRK3-HOMER2.

In another aspect, provided herein is a method of determining if a subject has cancer or is likely to develop cancer, comprising: (i) obtaining a sample from the subject; and (ii) detecting a NTRK fusion gene in the sample by RNA in situ hybridization using a probe comprising a nucleic acid sequence complementary to a region of mRNA encoding a kinase domain of NTRK1, NTRK2, or NTRK3.

In some embodiments, the probe comprises a nucleic acid sequence complementary to a region of the mRNA encoding the kinase domain of NTRK1. In one embodiment, the probe comprises a nucleic acid sequence complementary to a region within SEQ ID NO:3, SEQ ID NO:5, SEQ ID NO:6, or SEQ ID NO:7.

In some embodiments, the probe comprises a nucleic acid sequence complementary to a region of the mRNA encoding the kinase domain of NTRK2. In one embodiment, the probe comprises a nucleic acid sequence complementary to a region within SEQ ID NO:10, SEQ ID NO:12, SEQ ID NO:13 or SEQ ID NO:14.

In some embodiments, the probe comprises a nucleic acid sequence complementary to a region of the mRNA encoding the kinase domain of NTRK3. In one embodiment, the probe comprises a nucleic acid sequence complementary to a region within SEQ ID NO:17, SEQ ID NO:19, SEQ ID NO:20 or SEQ ID NO:21.

In some embodiments, the method comprises detecting a NTRK fusion gene in the sample by RNA in situ hybridization using a probe pool comprising at least two of (a) a first probe comprising a nucleic acid sequence complementary to a region of the mRNA encoding the kinase domain of NTRK1; (b) a second probe comprising a nucleic acid sequence complementary to a region of the mRNA encoding the kinase domain of NTRK2; and (c) a third probe comprising a nucleic acid sequence complementary to a region of the mRNA encoding the kinase domain of NTRK3. In one embodiment, the probe pool comprises the first probe comprising a nucleic acid sequence complementary to a region of the mRNA encoding the kinase domain of NTRK1; and the second probe comprising a nucleic acid sequence complementary to a region of the mRNA encoding the kinase domain of NTRK2. In another embodiment, the probe pool comprises the first probe comprising a nucleic acid sequence complementary to a region of the mRNA encoding the kinase domain of NTRK1; and the third probe comprising a nucleic acid sequence complementary to a region of the mRNA encoding the kinase domain of NTRK3. In yet another embodiment, the probe pool comprises the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3. In still another embodiment, the probe pool comprises the first probe comprising a nucleic acid sequence complementary to a region of the mRNA encoding the kinase domain of NTRK1; the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3. In some embodiments, the first probe comprises a nucleic acid sequence complementary to a region within SEQ ID NO:3, SEQ ID NO:5, SEQ ID NO:6, or SEQ ID NO:7, the second probe comprises a nucleic acid sequence complementary to a region within SEQ ID NO:10, SEQ ID NO:12, SEQ ID NO:13, or SEQ ID NO:14, and the third probe comprises a nucleic acid sequence complementary to a region within SEQ ID NO:17, SEQ ID NO:19, SEQ ID NO:20, or SEQ ID NO:21.

In some embodiments, the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, SQSTM1-NTRK2, ETV6-NTRK3, BTBD1-NTRK3, EML4-NTRK3, TFG-NTRK3, RBPMS-NTRK3, LYN-NTRK3, and NTRK3-HOMER2. In specific embodiments, the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, and MRPL24-NTRK1. In other specific embodiments, the NTRK fusion is selected from a group consisting of OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, and SQSTM1-NTRK2. In yet other specific embodiments, the NTRK fusion is selected from a group consisting of ETV6-NTRK3, BTBD1-NTRK3, EML4-NTRK3, TFG-NTRK3, RBPMS-NTRK3, LYN-NTRK3, and NTRK3-HOMER2.

In some embodiments, the cancer is associated with NTRK fusion. In specific embodiments, the cancer is selected from a group consisting of colorectal cancer (CRC), papillary thyroid cancer (PTC), non-small-cell lung carcinoma (NSCLC), sarcoma, pediatric glioma, breast cancer, gallbladder, cholangiocarcinoma, spitzoid melanoma, astrocytoma, glioblastoma (GBM), pancreatic cancer, uterus carcinoma, pilocytic astrocytoma, pediatric glioma, head and neck squamous cell carcinoma (HNSCC), glioma, salivary gland tumor (including acinic cell carcinoma), adult acute myeloid leukemia (AML), nephroma, and inflammatory myofibroblastic tumor (IMT).

In some embodiments, the presence of the NTRK fusion gene indicates that the subject has cancer or is likely to develop cancer. In some embodiments, the method further comprises administering a treatment compound to the subject in whom the NTRK fusion gene is detected. In some embodiments, the treatment compound is a NTRK kinase inhibitor. In specific embodiments, the treatment compound is selected from a group consisting of larotrectinib, entrectinib, TPX-0005/repotrectinib, crizotinib, cabozantinib, altiratinib, foretinib, ponatinib, nintedanib, merestinib, BAY2731954 (LOXO-195), MGCD516, PLX7486, DS-6051b, and TSR-011. In one embodiment, the treatment compound is larotrectinib. In another embodiment, the treatment compound is entrectinib.

In yet another aspect, provided herein is a method of treating cancer comprising administering a NTRK kinase inhibitor to a subject determined to have a NTRK fusion gene using a method comprising: (i) obtaining a sample from the subject; and (ii) detecting a NTRK fusion gene in the sample by RNA in situ hybridization using a probe comprising a nucleic acid sequence complementary to a region of mRNA encoding a kinase domain of NTRK1, NTRK2, or NTRK3.

In some embodiments, the probe comprises a nucleic acid sequence complementary to a region of the mRNA encoding the kinase domain of NTRK1. In one embodiment, the probe comprises a nucleic acid sequence complementary to a region within SEQ ID NO:3, SEQ ID NO:5, SEQ ID NO:6, or SEQ ID NO:7.

In some embodiments, the probe comprises a nucleic acid sequence complementary to a region of the mRNA encoding the kinase domain of NTRK2. In one embodiment, the probe comprises a nucleic acid sequence complementary to a region within SEQ ID NO:10, SEQ ID NO:12, SEQ ID NO:13 or SEQ ID NO:14.

In some embodiments, the probe comprises a nucleic acid sequence complementary to a region of the mRNA encoding the kinase domain of NTRK3. In one embodiment, the probe comprises a nucleic acid sequence complementary to a region within SEQ ID NO:17, SEQ ID NO:19, SEQ ID NO:20 or SEQ ID NO:21.

In some embodiments, the method comprises detecting a NTRK fusion gene in the sample by RNA in situ hybridization using a probe pool comprising at least two of (a) a first probe comprising a nucleic acid sequence complementary to a region of the mRNA encoding the kinase domain of NTRK1; (b) a second probe comprising a nucleic acid sequence complementary to a region of the mRNA encoding the kinase domain of NTRK2; and (c) a third probe comprising a nucleic acid sequence complementary to a region of the mRNA encoding the kinase domain of NTRK3. In one embodiment, the probe pool comprises the first probe comprising a nucleic acid sequence complementary to a region of the mRNA encoding the kinase domain of NTRK1; and the second probe comprising a nucleic acid sequence complementary to a region of the mRNA encoding the kinase domain of NTRK2. In another embodiment, the probe pool comprises the first probe comprising a nucleic acid sequence complementary to a region of the mRNA encoding the kinase domain of NTRK1; and the third probe comprising a nucleic acid sequence complementary to a region of the mRNA encoding the kinase domain of NTRK3. In yet another embodiment, the probe pool comprises the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3. In still another embodiment, the probe pool comprises the first probe comprising a nucleic acid sequence complementary to a region of the mRNA encoding the kinase domain of NTRK1; the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3. In some embodiments, the first probe comprises a nucleic acid sequence complementary to a region within SEQ ID NO:3, SEQ ID NO:5, SEQ ID NO:6, or SEQ ID NO:7, the second probe comprises a nucleic acid sequence complementary to a region within SEQ ID NO:10, SEQ ID NO:12, SEQ ID NO:13, or SEQ ID NO:14, and the third probe comprises a nucleic acid sequence complementary to a region within SEQ ID NO:17, SEQ ID NO:19, SEQ ID NO:20, or SEQ ID NO:21.

In some embodiments, the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, SQSTM1-NTRK2, ETV6-NTRK3, BTBD1-NTRK3, EML4-NTRK3, TFG-NTRK3, RBPMS-NTRK3, LYN-NTRK3, and NTRK3-HOMER2. In specific embodiments, the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, and MRPL24-NTRK1. In other specific embodiments, the NTRK fusion is selected from a group consisting of OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, and SQSTM1-NTRK2. In yet other specific embodiments, the NTRK fusion is selected from a group consisting of ETV6-NTRK3, BTBD1-NTRK3, EML4-NTRK3, TFG-NTRK3, RBPMS-NTRK3, LYN-NTRK3, and NTRK3-HOMER2.

In some embodiments, the cancer is associated with NTRK fusion. In specific embodiments, the cancer is selected from a group consisting of colorectal cancer (CRC), papillary thyroid cancer (PTC), non-small-cell lung carcinoma (NSCLC), sarcoma, pediatric glioma, breast cancer, gallbladder, cholangiocarcinoma, spitzoid melanoma, astrocytoma, glioblastoma (GBM), pancreatic cancer, uterus carcinoma, pilocytic astrocytoma, pediatric glioma, head and neck squamous cell carcinoma (HNSCC), glioma, salivary gland tumor (including acinic cell carcinoma), adult acute myeloid leukemia (AML), nephroma, and inflammatory myofibroblastic tumor (IMT).

In some embodiments, the presence of the NTRK fusion gene indicates that the subject has cancer or is likely to develop cancer. In some embodiments, the method further comprises administering a treatment compound to the subject in whom the NTRK fusion gene is detected. In some embodiments, the treatment compound is a NTRK kinase inhibitor. In specific embodiments, the treatment compound is selected from a group consisting of larotrectinib, entrectinib, TPX-0005/repotrectinib, crizotinib, cabozantinib, altiratinib, foretinib, ponatinib, nintedanib, merestinib, BAY2731954 (LOXO-195), MGCD516, PLX7486, DS-6051b, and TSR-011. In one embodiment, the treatment compound is larotrectinib. In another embodiment, the treatment compound is entrectinib.

In some embodiments, the sample is a tissue specimen or is derived from a tissue specimen. In some embodiments, the tissue specimen is formalin-fixed paraffin-embedded (FFPE). In some embodiments, the tissue specimen is fresh frozen. In some embodiments, the tissue specimen is prepared with a fixative other than formalin. In some embodiments, the fixative other than formalin is selected from the group consisting of ethanol, methanol, Bouin's, B5, and I.B.F. In some embodiments, the sample is a blood sample or is derived from a blood sample. In other embodiments, the sample is a cytological sample or is derived from a cytological sample. In yet other embodiments, the sample is a tumor sample. In still yet other embodiments, the sample is processed for RNA in situ hybridization. In some embodiments, the sample is paraffin embedded and/or formalin fixed.

In some embodiments, the probe is entirely complementary to the region of mRNA encoding a kinase domain of NTRK1, NTRK2, or NTRK3. In some embodiments, the probe further comprises a nucleic acid sequence not complementary to the region of mRNA encoding a kinase domain of NTRK1, NTRK2, or NTRK3. In other embodiments, a 3′ region of the probe is complementary to the region of mRNA encoding a kinase domain of NTRK1, NTRK2, or NTRK3. In yet other embodiments, a 5′ region of the probe is complementary to the region of mRNA encoding a kinase domain of NTRK1, NTRK2, or NTRK3.

In yet another aspect, provided herein is a kit for detecting a neurotrophic tyrosine receptor kinase (NTRK) fusion gene, comprising an agent for detecting a NTRK fusion gene in the sample by RNA in situ hybridization. In some embodiments, the agent comprises a probe comprising a nucleic acid sequence complementary to a region of mRNA encoding a kinase domain of NTRK1, NTRK2, or NTRK3.

In some embodiments, the probe comprises a nucleic acid sequence complementary to a region of the mRNA encoding the kinase domain of NTRK1. In one embodiment, the probe comprises a nucleic acid sequence complementary to a region within SEQ ID NO:3, SEQ ID NO:5, SEQ ID NO:6, or SEQ ID NO:7.

In some embodiments, the probe comprises a nucleic acid sequence complementary to a region of the mRNA encoding the kinase domain of NTRK2. In one embodiment, the probe comprises a nucleic acid sequence complementary to a region within SEQ ID NO:10, SEQ ID NO:12, SEQ ID NO:13 or SEQ ID NO:14.

In some embodiments, the probe comprises a nucleic acid sequence complementary to a region of the mRNA encoding the kinase domain of NTRK3. In one embodiment, the probe comprises a nucleic acid sequence complementary to a region within SEQ ID NO:17, SEQ ID NO:19, SEQ ID NO:20 or SEQ ID NO:21.

In some embodiments, the method comprises detecting a NTRK fusion gene in the sample by RNA in situ hybridization using a probe pool comprising at least two of (a) a first probe comprising a nucleic acid sequence complementary to a region of the mRNA encoding the kinase domain of NTRK1; (b) a second probe comprising a nucleic acid sequence complementary to a region of the mRNA encoding the kinase domain of NTRK2; and (c) a third probe comprising a nucleic acid sequence complementary to a region of the mRNA encoding the kinase domain of NTRK3. In one embodiment, the probe pool comprises the first probe comprising a nucleic acid sequence complementary to a region of the mRNA encoding the kinase domain of NTRK1; and the second probe comprising a nucleic acid sequence complementary to a region of the mRNA encoding the kinase domain of NTRK2. In another embodiment, the probe pool comprises the first probe comprising a nucleic acid sequence complementary to a region of the mRNA encoding the kinase domain of NTRK1; and the third probe comprising a nucleic acid sequence complementary to a region of the mRNA encoding the kinase domain of NTRK3. In yet another embodiment, the probe pool comprises the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3. In still another embodiment, the probe pool comprises the first probe comprising a nucleic acid sequence complementary to a region of the mRNA encoding the kinase domain of NTRK1; the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3. In some embodiments, the first probe comprises a nucleic acid sequence complementary to a region within SEQ ID NO:3, SEQ ID NO:5, SEQ ID NO:6, or SEQ ID NO:7, the second probe comprises a nucleic acid sequence complementary to a region within SEQ ID NO:10, SEQ ID NO:12, SEQ ID NO:13, or SEQ ID NO:14, and the third probe comprises a nucleic acid sequence complementary to a region within SEQ ID NO:17, SEQ ID NO:19, SEQ ID NO:20, or SEQ ID NO:21.

In some embodiments, the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, SQSTM1-NTRK2, ETV6-NTRK3, BTBD1-NTRK3, EML4-NTRK3, TFG-NTRK3, RBPMS-NTRK3, LYN-NTRK3, and NTRK3-HOMER2. In specific embodiments, the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, and MRPL24-NTRK1. In other specific embodiments, the NTRK fusion is selected from a group consisting of OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, and SQSTM1-NTRK2. In yet other specific embodiments, the NTRK fusion is selected from a group consisting of ETV6-NTRK3, BTBD1-NTRK3, EML4-NTRK3, TFG-NTRK3, RBPMS-NTRK3, LYN-NTRK3, and NTRK3-HOMER2.

In some embodiments, the probe is entirely complementary to the region of mRNA encoding a kinase domain of NTRK1, NTRK2, or NTRK3. In some embodiments, the probe further comprises a nucleic acid sequence not complementary to the region of mRNA encoding a kinase domain of NTRK1, NTRK2, or NTRK3. In other embodiments, a 3′ region of the probe is complementary to the region of mRNA encoding a kinase domain of NTRK1, NTRK2, or NTRK3. In yet other embodiments, a 5′ region of the probe is complementary to the region of mRNA encoding a kinase domain of NTRK1, NTRK2, or NTRK3.

### 6. BRIEF DESCRIPTION OF THE DRAWINGS

FIGS. 1A-1C: Representative images of sample sections obtained from a patient's thyroid gland (Table 1, Case 1-7) and stained for hematoxylin-eosin (A), pan-TRK immunohistochemistry (B), and pan-NTRK in situ hybridization (C).

FIGS. 2A-2C: Representative images of sample sections obtained from a patient's thyroid gland (Table 1, Case 1-6) and stained for hematoxylin-eosin (A), pan-TRK immunohistochemistry (B), and pan-NTRK in situ hybridization (C).

FIGS. 3A-3C: Representative images of sample sections obtained from a patient's thyroid gland (Table 1, Case 1-7) and stained for hematoxylin-eosin (A), pan-TRK immunohistochemistry (B), and pan-NTRK in situ hybridization (C).

FIG. 4: Representative schematic diagram of a NTRK1/2/3 gene fusion detection method workflow, according to one embodiment of the present disclosure.

### 7. DETAILED DESCRIPTION

### 7.1. Definitions

As used herein, the term “cancer” refers to all neoplastic cell growth and proliferation. The term “cancer” includes, but is not limited to, solid cancer and blood borne cancer. The term “cancer” refers to disease of tissues or organs, including but not limited to, cancers of the bladder, bone, blood, brain, breast, cervix, chest, colon, endometrium, esophagus, eye, head, kidney, liver, lymph nodes, lung, mouth, neck, ovaries, pancreas, prostate, rectum, skin, stomach, testis, throat, and uterus. Specific cancers include, but are not limited to, advanced malignancy, amyloidosis, neuroblastoma, meningioma, hemangiopericytoma, multiple brain metastases, glioblastoma multiforme, glioblastoma, brain stem glioma, poor prognosis malignant brain tumor, malignant glioma, recurrent malignant glioma, anaplastic astrocytoma, anaplastic oligodendroglioma, neuroendocrine tumor, rectal adenocarcinoma, colorectal cancer, including stage 3 and stage 4 colorectal cancer, unresectable colorectal carcinoma, metastatic hepatocellular carcinoma, Kaposi's sarcoma, karyotype acute myeloblastic leukemia, Hodgkin's lymphoma, non-Hodgkin's lymphoma, cutaneous T-Cell lymphoma, cutaneous B-Cell lymphoma, diffuse large B-Cell lymphoma, low grade follicular lymphoma, malignant melanoma, malignant mesothelioma, malignant pleural effusion mesothelioma syndrome, peritoneal carcinoma, papillary serous carcinoma, gynecologic sarcoma, soft tissue sarcoma, scleroderma, cutaneous vasculitis, Langerhans cell histiocytosis, leiomyosarcoma, fibrodysplasia ossificans progressive, hormone refractory prostate cancer, resected high-risk soft tissue sarcoma, unresectable hepatocellular carcinoma, Waldenstrom's macroglobulinemia, smoldering myeloma, indolent myeloma, fallopian tube cancer, androgen independent prostate cancer, androgen dependent stage IV non-metastatic prostate cancer, hormone-insensitive prostate cancer, chemotherapy-insensitive prostate cancer, papillary thyroid carcinoma, follicular thyroid carcinoma, medullary thyroid carcinoma, and leiomyoma.

As used herein, and unless otherwise specified, the terms “treat,” “treating,” and “treatment” refer to an action that occurs while a patient is suffering from the specified cancer, which reduces the severity of the cancer or retards or slows the progression of the cancer.

As used herein, the terms “compound” and “treatment compound” are used interchangeably. Non-limiting examples of compounds include those disclosed in Section 7.5 below.

As used herein, and unless otherwise specified, the term “therapeutically effective amount” of a compound is an amount sufficient to provide a therapeutic benefit in the treatment or management of a cancer, or to delay or minimize one or more symptoms associated with the presence of the cancer. A therapeutically effective amount of a compound means an amount of therapeutic agent, alone or in combination with other therapies, which provides a therapeutic benefit in the treatment or management of the cancer. The term “therapeutically effective amount” can encompass an amount that improves overall therapy, reduces or avoids symptoms or causes of cancer, or enhances the therapeutic efficacy of another therapeutic agent. The term also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician. An improvement in the cancer or cancer-related disease can be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about 10%, about 20%, about 30%, about 40%, about 50%, about 60%, about 70%, about 80%, or about 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

The terms “detecting” as used herein generally refer to any form of measurement, and include determining whether an element is present or not. This term includes quantitative and/or qualitative determinations.

The terms “nucleic acid” and “polynucleotide” are used interchangeably herein to describe a polymer of any length composed of nucleotides, e.g., deoxyribonucleotides or ribonucleotides, or compounds produced synthetically, which can hybridize with naturally occurring nucleic acids in a sequence specific manner analogous to that of two naturally occurring nucleic acids, e.g., can participate in Watson-Crick base pairing interactions. As used herein in the context of a polynucleotide sequence, the term “bases” (or “base”) is synonymous with “nucleotides” (or “nucleotide”), i.e., the monomer subunit of a polynucleotide. The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like. “Analogues” refer to molecules having structural features that are recognized in the literature as being mimetics, derivatives, having analogous structures, or other like terms, and include, for example, polynucleotides incorporating non-natural nucleotides, nucleotide mimetics such as 2′-modified nucleosides, peptide nucleic acids, oligomeric nucleoside phosphonates, and any polynucleotide that has added substituent groups, such as protecting groups or linking moieties.

The term “complementary” refers to specific binding between polynucleotides based on the sequences of the polynucleotides. As used herein, a first polynucleotide and a second polynucleotide are complementary if they bind to each other in a hybridization assay under stringent conditions, e.g., if they produce a given or detectable level of signal in a hybridization assay. Portions of polynucleotides are complementary to each other if they follow conventional base-pairing rules, e.g., A pairs with T (or U) and G pairs with C, although small regions (e.g., fewer than about 3 bases) of mismatch, insertion, or deleted sequence may be present.

The term “sample” as used herein relates to a material or mixture of materials containing one or more components of interest. The term “sample” includes “biological sample” which refers to a sample obtained from a biological subject, including a sample of biological tissue or fluid origin, obtained, reached, or collected in vivo or in situ. A biological sample also includes samples from a region of a biological subject containing precancerous or cancer cells or tissues. Such samples can be, but are not limited to, organs, tissues, and cells isolated from a mammal. Exemplary biological samples include but are not limited to cell lysate, a cell culture, a cell line, a tissue, oral tissue, gastrointestinal tissue, an organ, an organelle, a biological fluid, a blood sample, a urine sample, a skin sample, and the like. Preferred biological samples include, but are not limited to, whole blood, partially purified blood, PBMC, tissue biopsies, and the like.

The term “probe” as used herein refers to a capture agent that is directed to a specific target mRNA sequence. Accordingly, each probe of a probe set has a respective target mRNA sequence. In some embodiments, the probe provided herein is a “nucleic acid probe” or “oligonucleotide probe” which refers to a nucleic acid capable of binding to a target nucleic acid of complementary sequence, such as the mRNA biomarkers provided herein, usually through complementary base pairing by forming hydrogen bond. As used herein, a probe may include natural (e.g., A, G, C, or T) or modified bases (7-deazaguanosine, inosine, etc.). In addition, the bases in a probe may be joined by a linkage other than a phosphodiester bond, so long as it does not interfere with hybridization. The probes can be directly or indirectly labeled with tags, for example, chromophores, lumiphores, or chromogens. By assaying for the presence or absence of the probe, one can detect the presence or absence of a target mRNA biomarker of interest.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 7.2. NTRK Gene Fusion

The present disclosure relates to methods for detecting NTRK gene fusion by RNA in situ hybridization, and uses and kits thereof.

The NTRK family is a family of transmembrane tyrosine kinases that are important players in neural development. The three members of the family, TRKA, TRKB and TRKC, are encoded by the NTRK1, NTRK2 and NTRK3 genes, respectively, and each consists of an extracellular ligand-binding domain, a transmembrane region and an intracellular kinase domain. The amino acid sequences and mRNA sequences of TRKA, TRKB and TRKC and their kinase domains are as shown in the table below:

| SEQ ID # | Sequence | Name |
|---|---|---|
| SEQ ID NO: 1 | TGCAGCTGGGAGCGCACAGACGGCTGCCCCGCCTGA | NTRK1 full |
| | GCGAGGCGGGCGCCGCCGCGATGCTGCGAGGCGG | mRNA |
| | ACGGCGCGGGCAGCTTGGCTGGCACAGCTGGGCTGC | |
| | GGGGCCGGGCAGCCTGCTGGCTTGGCTGATACTG | |
| | GCATCTGCGGGCGCCGCACCCTGCCCCGATGCCTGCT | |
| | GCCCCCACGGCTCCTCGGGACTGCGATGCACCC | |
| | GGGATGGGGCCCTGGATAGCCTCCACCACCTGCCCGG | |
| | CGCAGAGAACCTGACTGAGCTCTACATCGAGAA | |
| | CCAGCAGCATCTGCAGCATCTGGAGCTCCGTGATCTG | |
| | AGGGGCCTGGGGGAGCTGAGAAACCTCACCATC | |
| | GTGAAGAGTGGTCTCCGTTTCGTGGCGCCAGATGCCT | |
| | TCCATTTCACTCCTCGGCTCAGTCGCCTGAATC | |
| | TCTCCTTCAACGCTCTGGAGTCTCTCTCCTGGAAAACT | |
| | GTGCAGGGCCTCTCCTTACAGGAACTGGTCCT | |
| | GTCGGGGAACCCTCTGCACTGTTCTTGTGCCCTGCGCT | |
| | GGCTACAGCGCTGGGAGGAGGAGGGACTGGGC | |
| | GGAGTGCCTGAACAGAAGCTGCAGTGTCATGGGCAA | |
| | GGGCCCCTGGCCCACATGCCCAATGCCAGCTGTG | |
| | GTGTGCCCACGCTGAAGGTCCAGGTGCCCAATGCCTC | |
| | GGTGGATGTGGGGGACGACGTGCTGCTGCGGTG | |
| | CCAGGTGGAGGGGCGGGGCCTGGAGCAGGCCGGCTG | |
| | GATCCTCACAGAGCTGGAGCAGTCAGCCACGGTG | |
| | ATGAAATCTGGGGGTCTGCCATCCCTGGGGCTGACCC | |
| | TGGCCAATGTCACCAGTGACCTCAACAGGAAGA | |
| | ACGTGACGTGCTGGGCAGAGAACGATGTGGGCCGGG | |
| | CAGAGGTCTCTGTTCAGGTCAACGTCTCCTTCCC | |
| | GGCCAGTGTGCAGCTGCACACGGCGGTGGAGATGCA | |
| | CCACTGGTGCATCCCCTTCTCTGTGGATGGGCAG | |
| | CCGGCACCGTCTCTGCGCTGGCTCTTCAATGGCTCCGT | |
| | GCTCAATGAGACCAGCTTCATCTTCACTGAGT | |
| | TCCTGGAGCCGGCAGCCAATGAGACCGTGCGGCACG | |
| | GGTGTCTGCGCCTCAACCAGCCCACCCACGTCAA | |
| | CAACGGCAACTACACGCTGCTGGCTGCCAACCCCTTC | |
| | GGCCAGGCCTCCGCCTCCATCATGGCTGCCTTC | |
| | ATGGACAACCCTTTCGAGTTCAACCCCGAGGACCCCA | |
| | TCCCTGACACTAACAGCACATCTGGAGACCCGG | |
| | TGGAGAAGAAGGACGAAACACCTTTTGGGGTCTCGGT | |
| | GGCTGTGGGCCTGGCCGTCTTTGCCTGCCTCTT | |
| | CCTTTCTACGCTGCTCCTTGTGCTCAACAAATGTGGAC | |
| | GGAGAAACAAGTTTGGGATCAACCGCCCGGCT | |
| | GTGCTGGCTCCAGAGGATGGGCTGGCCATGTCCCTGC | |
| | ATTTCATGACATTGGGTGGCAGCTCCCTGTCCC | |
| | CCACCGAGGGCAAAGGCTCTGGGCTCCAAGGCCACA | |
| | TCATCGAGAACCCACAATACTTCAGTGATGCCTG | |
| | TGTTCACCACATCAAGCGCCGGGACATCGTGCTCAAG | |
| | TGGGAGCTGGGGGAGGGCGCCTTTGGGAAGGTC | |
| | TTCCTTGCTGAGTGCCACAACCTCCTGCCTGAGCAGG | |
| | ACAAGATGCTGGTGGCTGTCAAGGCACTGAAGG | |
| | AGGCGTCCGAGAGTGCTCGGCAGGACTTCCAGCGTGA | |
| | GGCTGAGCTGCTCACCATGCTGCAGCACCAGCA | |
| | CATCGTGCGCTTCTTCGGCGTCTGCACCGAGGGCCGC | |
| | CCCCTGCTCATGGTCTTTGAGTATATGCGGCAC | |
| | GGGGACCTCAACCGCTTCCTCCGATCCCATGGACCTG | |
| | ATGCCAAGCTGCTGGCTGGTGGGGAGGATGTGG | |
| | CTCCAGGCCCCCTGGGTCTGGGGCAGCTGCTGGCCGT | |
| | GGCTAGCCAGGTCGCTGCGGGGATGGTGTACCT | |
| | GGCGGGTCTGCATTTTGTGCACCGGGACCTGGCCACA | |
| | CGCAACTGTCTAGTGGGCCAGGGACTGGTGGTC | |
| | AAGATTGGTGATTTTGGCATGAGCAGGGATATCTACA | |
| | GCACCGACTATTACCGTGTGGGAGGCCGCACCA | |
| | TGCTGCCCATTCGCTGGATGCCGCCCGAGAGCATCCT | |
| | GTACCGTAAGTTCACCACCGAGAGCGACGTGTG | |
| | GAGCTTCGGCGTGGTGCTCTGGGAGATCTTCACCTAC | |
| | GGCAAGCAGCCCTGGTACCAGCTCTCCAACACG | |
| | GAGGCAATCGACTGCATCACGCAGGGACGTGAGTTG | |
| | GAGCGGCCACGTGCCTGCCCACCAGAGGTCTACG | |
| | CCATCATGCGGGGCTGCTGGCAGCGGGAGCCCCAGC | |
| | AACGCCACAGCATCAAGGATGTGCACGCCCGGCT | |
| | GCAAGCCCTGGCCCAGGCACCTCCTGTCTACCTGGAT | |
| | GTCCTGGGCTAGGGGGCCGGCCCAGGGGCTGGG | |
| | AGTGGTTAGCCGGAATACTGGGGCCTGCCCTCAGCAT | |
| | CCCCCATAGCTCCCAGCAGCCCCAGGGTGATCT | |
| | CAAAGTATCTAATTCACCCTCAGCATGTGGGAAGGGA | |
| | CAGGTGGGGGCTGGGAGTAGAGGATGTTCCTGC | |
| | TTCTCTAGGCAAGGTCCCGTCATAGCAATTATATTTAT | |
| | TATCCCTTGAAAAAAAAAA | |
| SEQ ID NO: 2 | MLRGGRRGQLGWHSWAAGPGSLLAWLILASAGAAPC | NTRK1 full |
| | PDACCPHGSSGLRCTRDGALDSLHHLPGAENLTELYIE | amino acid |
| | NQQHLQHLELRDLRGLGELRNLTIVKSGLRFVAPDAFH | |
| | FTPRLSRLNLSFNALESLSWKTVQGLSLQELVLSGNPLH | |
| | CSCALRWLQRWEEEGLGGVPEQKLQCHGQGPLAHMP | |
| | NASCGVPTLKVQVPNASVDVGDDVLLRCQVEGRGLEQ | |
| | AGWILTELEQSATVMKSGGLPSLGLTLANVTSDLNRKN | |
| | VTCWAENDVGRAEVSVQVNVSFPASVQLHTAVEMHH | |
| | WCIPFSVDGQPAPSLRWLFNGSVLNETSFIFTEFLEPAA | |
| | NETVRHGCLRLNQPTHVNNGNYTLLAANPFGQASASI | |
| | MAAFMDNPFEFNPEDPIPDTNSTSGDPVEKKDETPFGVS | |
| | VAVGLAVFACLFLSTLLLVLNKCGRRNKFGINRPAVLA | |
| | PEDGLAMSLHFMTLGGSSLSPTEGKGSGLQGHIIENPQY | |
| | FSDACVHHIKRRDIVLKWELGEGAFGKVFLAECHNLLP | |
| | EQDKMLVAVKALKEASESARQDFQREAELLTMLQHQH | |
| | IVRFFGVCTEGRPLLMVFEYMRHGDLNRFLRSHGPDAK | |
| | LLAGGEDVAPGPLGLGQLLAVASQVAAGMVYLAGLHF | |
| | VHRDLATRNCLVGQGLVVKIGDFGMSRDIYSTDYYRV | |
| | GGRTMLPIRWMPPESILYRKFTTESDVWSFGVVLWEIFT | |
| | YGKQPWYQLSNTEAIDCITQGRELERPRACPPEVYAIM | |
| | RGCWQREPQQRHSIKDVHARLQALAQAPPVYLDVLG | |
| SEQ ID NO: 3 | ATCGTGCTCAAGTGGGAGCTGGGGGAGGGCGCCTTT | NTRK1 kinase |
| | GGGAAGGTCTTCCTTGCTGAGTGCCACAACCTCCTGC | domain mRNA |
| | CTGAGCAGGACAAGATGCTGGTGGCTGTCAAGGCAC | |
| | TGAAGGAGGCGTCCGAGAGTGCTCGGCAGGACTTCC | |
| | AGCGTGAGGCTGAGCTGCTCACCATGCTGCAGCACC | |
| | AGCACATCGTGCGCTTCTTCGGCGTCTGCACCGAGGG | |
| | CCGCCCCCTGCTCATGGTCTTTGAGTATATGCGGCAC | |
| | GGGGACCTCAACCGCTTCCTCCGATCCCATGGACCTG | |
| | ATGCCAAGCTGCTGGCTGGTGGGGAGGATGTGGCTC | |
| | CAGGCCCCCTGGGTCTGGGGCAGCTGCTGGCCGTGG | |
| | CTAGCCAGGTCGCTGCGGGGATGGTGTACCTGGCGG | |
| | GTCTGCATTTTGTGCACCGGGACCTGGCCACACGCAA | |
| | CTGTCTAGTGGGCCAGGGACTGGTGGTCAAGATTGG | |
| | TGATTTTGGCATGAGCAGGGATATCTACAGCACCGA | |
| | CTATTACCGTGTGGGAGGCCGCACCATGCTGCCCATT | |
| | CGCTGGATGCCGCCCGAGAGCATCCTGTACCGTAAG | |
| | TTCACCACCGAGAGCGACGTGTGGAGCTTCGGCGTG | |
| | GTGCTCTGGGAGATCTTCACCTACGGCAAGCAGCCCT | |
| | GGTACCAGCTCTCCAACACGGAGGCAATCGACTGCA | |
| | TCACGCAGGGACGTGAGTTGGAGCGGCCACGTGCCT | |
| | GCCCACCAGAGGTCTACGCCATCATGCGGGGCTGCT | |
| | GGCAGCGGGAGCCCCAGCAACGCCACAGCATCAAGG | |
| | ATGTGCACGCCCGG | |
| SEQ ID NO: 4 | IVLKWELGEGAFGKVFLAECHNLLPEQDKMLVAVKAL | NTRK1 kinase |
| | KEASESARQDFQREAELLTMLQHQHIVRFFGVCTEGRP | domain amino |
| | LLMVFEYMRHGDLNRFLRSHGPDAKLLAGGEDVAPGP | acid |
| | LGLGQLLAVASQVAAGMVYLAGLHFVHRDLATRNCL | |
| | VGQGLVVKIGDFGMSRDIYSTDYYRVGGRTMLPIRWM | |
| | PPESILYRKFTTESDVWSFGVVLWEIFTYGKQPWYQLS | |
| | NTEAIDCITQGRELERPRACPPEVYAIMRGCWQREPQQ | |
| | RHSIKDVHAR | |
| SEQ ID NO: 8 | ACCAGAGCCCTCGGAAGTGTCAGGCACTGCGGTGTA | NTRK2 full |
| | TTTTCCCCGTTCTCTTTTAAATGACTGCGGAAAA | mRNA |
| | ACAGATTCCGAGCCGCAAAAGGGAAGACGGATTCTC | |
| | AGACAAGGCTTGCAAATGCCCCGCAGCCATCATT | |
| | TAACTGCACCCGCAGAATAGTTACGGTTTGTCACCCG | |
| | ACCCTCCCGGATCGCCTAATTTGTCCCTAGTGA | |
| | GACCCCGAGGCTCTGCCCGCGCCTGGCTTCTTCGTAG | |
| | CTGGATGCATATCGTGCTCCGGGCAGCGCGGGC | |
| | GCAGGGCACGCGTTCGCGCACACCCTAGCACACATG | |
| | AACACGCGCAAGAGCTGAACCAAGCACGGTTTCC | |
| | ATTTCAAAAAGGGAGACAGCCTCTACCGCGATTGTA | |
| | GAAGAGACTGTGGTGTGAATTAGGGACCGGGAGG | |
| | CGTCGAACGGAGGAACGGTTCATCTTAGAGACTAAT | |
| | TTTCTGGAGTTTCTGCCCCTGCTCTGCGTCAGCC | |
| | CTCACGTCACTTCGCCAGCAGTAGCAGAGGCGGCGG | |
| | CGGCGGCTCCCGGAATTGGGTTGGAGCAGGAGCC | |
| | TCGCTGGCTGCTTCGCTCGCGCTCTACGCGCTCAGTC | |
| | CCCGGCGGTAGCAGGAGCCTGGACCCAGGCGCC | |
| | GCCGGCGGGCGTGAGGCGCCGGAGCCCGGCCTCGAG | |
| | GTGCATACCGGACCCCCATTCGCATCTAACAAGG | |
| | AATCTGCGCCCCAGAGAGTCCCGGGAGCGCCGCCGG | |
| | TCGGTGCCCGGCGCGCCGGGCCATGCAGCGACGG | |
| | CCGCCGCGGAGCTCCGAGCAGCGGTAGCGCCCCCCT | |
| | GTAAAGCGGTTCGCTATGCCGGGGCCACTGTGAA | |
| | CCCTGCCGCCTGCCGGAACACTCTTCGCTCCGGACCA | |
| | GCTCAGCCTCTGATAAGCTGGACTCGGCACGCC | |
| | CGCAACAAGCACCGAGGAGTTAAGAGAGCCGCAAGC | |
| | GCAGGGAAGGCCTCCCCGCACGGGTGGGGGAAAGCG | |
| | GCCGGTGCAGCGCGGGGACAGGCACTCGGGCTGGCA | |
| | CTGGCTGCTAGGGATGTCGTCCTGGATAAGGTGGCAT | |
| | GGACCCGCCATGGCGCGGCTCTGGGGCTTCTGCTGGC | |
| | TGGTTGTGGGCTTCTGGAGGGCCGCTTTCGCCTGTCC | |
| | CACGTCCTGCAAATGCAGTGCCTCTCGGATCTGGTGC | |
| | AGCGACCCTTCTCCTGGCATCGTG | |
| | GCATTTCCGAGATTGGAGCCTAACAGTGTAGATCCTG | |
| | AGAACATCACCGAAATTTTCATCGCAAACCAGA | |
| | AAAGGTTAGAAATCATCAACGAAGATGATGTTGAAG | |
| | CTTATGTGGGACTGAGAAATCTGACAATTGTGGA | |
| | TTCTGGATTAAAATTTGTGGCTCATAAAGCATTTCTG | |
| | AAAAACAGCAACCTGCAGCACATCAATTTTACC | |
| | CGAAACAAACTGACGAGTTTGTCTAGGAAACATTTC | |
| | CGTCACCTTGACTTGTCTGAACTGATCCTGGTGG | |
| | GCAATCCATTTACATGCTCCTGTGACATTATGTGGAT | |
| | CAAGACTCTCCAAGAGGCTAAATCCAGTCCAGA | |
| | CACTCAGGATTTGTACTGCCTGAATGAAAGCAGCAA | |
| | GAATATTCCCCTGGCAAACCTGCAGATACCCAAT | |
| | TGTGGTTTGCCATCTGCAAATCTGGCCGCACCTAACC | |
| | TCACTGTGGAGGAAGGAAAGTCTATCACATTAT | |
| | CCTGTAGTGTGGCAGGTGATCCGGTTCCTAATATGTA | |
| | TTGGGATGTTGGTAACCTGGTTTCCAAACATAT | |
| | GAATGAAACAAGCCACACACAGGGCTCCTTAAGGAT | |
| | AACTAACATTTCATCCGATGACAGTGGGAAGCAG | |
| | ATCTCTTGTGTGGCGGAAAATCTTGTAGGAGAAGATC | |
| | AAGATTCTGTCAACCTCACTGTGCATTTTGCAC | |
| | CAACTATCACATTTCTCGAATCTCCAACCTCAGACCA | |
| | CCACTGGTGCATTCCATTCACTGTGAAAGGCAA | |
| | CCCCAAACCAGCGCTTCAGTGGTTCTATAACGGGGC | |
| | AATATTGAATGAGTCCAAATACATCTGTACTAAA | |
| | ATACATGTTACCAATCACACGGAGTACCACGGCTGC | |
| | CTCCAGCTGGATAATCCCACTCACATGAACAATG | |
| | GGGACTACACTCTAATAGCCAAGAATGAGTATGGGA | |
| | AGGATGAGAAACAGATTTCTGCTCACTTCATGGG | |
| | CTGGCCTGGAATTGACGATGGTGCAAACCCAAATTA | |
| | TCCTGATGTAATTTATGAAGATTATGGAACTGCA | |
| | GCGAATGACATCGGGGACACCACGAACAGAAGTAAT | |
| | GAAATCCCTTCCACAGACGTCACTGATAAAACCGGT | |
| | CGGGAACATCTCTCGGTCTATGCTGTGGTGGTGATTG | |
| | CGTCTGTGGTGGGATTTTGCCTTTTGGTAAT | |
| | GCTGTTTCTGCTTAAGTTGGCAAGACACTCCAAGTTT | |
| | GGCATGAAAGATTTCTCATGGTTTGGATTTGGG | |
| | AAAGTAAAATCAAGACAAGGTGTTGGCCCAGCCTCC | |
| | GTTATCAGCAATGATGATGACTCTGCCAGCCCAC | |
| | TCCATCACATCTCCAATGGGAGTAACACTCCATCTTC | |
| | TTCGGAAGGTGGCCCAGATGCTGTCATTATTGG | |
| | AATGACCAAGATCCCTGTCATTGAAAATCCCCAGTAC | |
| | TTTGGCATCACCAACAGTCAGCTCAAGCCAGAC | |
| | ACATTTGTTCAGCACATCAAGCGACATAACATTGTTC | |
| | TGAAAAGGGAGCTAGGCGAAGGAGCCTTTGGAA | |
| | AAGTGTTCCTAGCTGAATGCTATAACCTCTGTCCTGA | |
| | GCAGGACAAGATCTTGGTGGCAGTGAAGACCCT | |
| | GAAGGATGCCAGTGACAATGCACGCAAGGACTTCCA | |
| | CCGTGAGGCCGAGCTCCTGACCAACCTCCAGCAT | |
| | GAGCACATCGTCAAGTTCTATGGCGTCTGCGTGGAG | |
| | GGCGACCCCCTCATCATGGTCTTTGAGTACATGA | |
| | AGCATGGGGACCTCAACAAGTTCCTCAGGGCACACG | |
| | GCCCTGATGCCGTGCTGATGGCTGAGGGCAACCC | |
| | GCCCACGGAACTGACGCAGTCGCAGATGCTGCATAT | |
| | AGCCCAGCAGATCGCCGCGGGCATGGTCTACCTG | |
| | GCGTCCCAGCACTTCGTGCACCGCGATTTGGCCACCA | |
| | GGAACTGCCTGGTCGGGGAGAACTTGCTGGTGA | |
| | AAATCGGGGACTTTGGGATGTCCCGGGACGTGTACA | |
| | GCACTGACTACTACAGGGTCGGTGGCCACACAAT | |
| | GCTGCCCATTCGCTGGATGCCTCCAGAGAGCATCATG | |
| | TACAGGAAATTCACGACGGAAAGCGACGTCTGG | |
| | AGCCTGGGGGTCGTGTTGTGGGAGATTTTCACCTATG | |
| | GCAAACAGCCCTGGTACCAGCTGTCAAACAATG | |
| | AGGTGATAGAGTGTATCACTCAGGGCCGAGTCCTGC | |
| | AGCGACCCCGCACGTGCCCCCAGGAGGTGTATGA | |
| | GCTGATGCTGGGGTGCTGGCAGCGAGAGCCCCACAT | |
| | GAGGAAGAACATCAAGGGCATCCATACCCTCCTT | |
| | CAGAACTTGGCCAAGGCATCTCCGGTCTACCTGGAC | |
| | ATTCTAGGCTAGGGCCCTTTTCCCCAGACCGATC | |
| | CTTCCCAACGTACTCCTCAGACGGGCTGAGAGGATG | |
| | AACATCTTTTAACTGCCGCTGGAGGCCACCAAGC | |
| | TGCTCTCCTTCACTCTGACAGTATTAACATCAAAGAC | |
| | TCCGAGAAGCTCTCGAGGGAAGCAGTGTGTACT | |
| | TCTTCATCCATAGACACAGTATTGACTTCTTTTTGGC | |
| | ATTATCTCTTTCTCTCTTTCCATCTCCCTTGGT | |
| | TGTTCCTTTTTCTTTTTTTAAATTTTCTTTTTCTTTTTTT | |
| | TTTCGTCTTCCCTGCTTCACGATTCTTACC | |
| | CTTTCTTTTGAATCAATCTGGCTTCTGCATTACTATTA | |
| | ACTCTGCATAGACAAAGGCCTTAACAAACGTA | |
| | ATTTGTTATATCAGCAGACACTCCAGTTTGCCCACCA | |
| | CAACTAACAATGCCTTGTTGTATTCCTGCCTTT | |
| | GATGTGGATGAAAAAAAGGGAAAACAAATATTTCAC | |
| | TTAAACTTTGTCACTTCTGCTGTACAGATATCGA | |
| | GAGTTTCTATGGATTCACTTCTATTTATTTATTATTAT | |
| | TACTGTTCTTATTGTTTTTGGATGGCTTAAGC | |
| | CTGTGTATAAAAAAGAAAACTTGTGTTCAATCTGTGA | |
| | AGCCTTTATCTATGGGAGATTAAAACCAGAGAG | |
| | AAAGAAGATTTATTATGAACCGCAATATGGGAGGAA | |
| | CAAAGACAACCACTGGGATCAGCTGGTGTCAGTC | |
| | CCTACTTAGGAAATACTCAGCAACTGTTAGCTGGGA | |
| | AGAATGTATTCGGCACCTTCCCCTGAGGACCTTT | |
| | CTGAGGAGTAAAAAGACTACTGGCCTCTGTGCCATG | |
| | GATGATTCTTTTCCCATCACCAGAAATGATAGCG | |
| | TGCAGTAGAGAGCAAAGATGGCTTCCGTGAGACACA | |
| | AGATGGCGCATAGTGTGCTCGGACACAGTTTTGT | |
| | CTTCGTAGGTTGTGATGATAGCACTGGTTTGTTTCTC | |
| | AAGCGCTATCCACAGAACCTTTGTCAACTTCAG | |
| | TTGAAAAGAGGTGGATTCATGTCCAGAGCTCATTTCG | |
| | GGGTCAGGTGGGAAAGCCAAGAACTTGGAAAAG | |
| | ATAAGACAAGCTATAAATTCGGAGGCAAGTTTCTTTT | |
| | ACAATGAACTTTTCAGATCTCACTTCCCTCCGA | |
| | CCCCTAACTTCCATGCCCACCCGTCCTTTTAACTGTG | |
| | CAAGCAAAATTGTGCATGGTCTTCGTCGATTAA | |
| | TACCTTGTGTGCAGACACTACTGCTCCAGACGTCGTT | |
| | TCCCTGATAGGTAGAGCAGATCCATAAAAAGGT | |
| | ATGACTTATACAATTAGGGGAAGCTAATGGAGTTTAT | |
| | TAGCTGAGTATCAATGTCTCTGCGTTGTACGGT | |
| | GGTGATGGGTTTTAATGAATATGGACCCTGAAGCCTG | |
| | GAAATCCTCATCCACGTCGAACCCACAGGACTG | |
| | TGGGAAGGGCAGAATCAATCCCTAAGGGAAAGGAA | |
| | ACCTCACCCTGAGGGCATCACATGCACTCATGTTC | |
| | AGTGTACACAGGTCAAGTCCCTTGCTCTGGGCTCTAG | |
| | TTGGGAGAGTGGTTTCATTCCAAGTGTACTCCA | |
| | TTGTCAGTATGCTGTTTTTGTTTCCTTCACTCCATTCA | |
| | AAAAGTCAAAATACAAAATTTGGCACAGCATG | |
| | CCAACGGGAGGCTGTGCCCAGACCAAGCACTGGAAG | |
| | TGTGCTTCTAGGCATAGTCATTGGTTTTGCAAAA | |
| | AGAGGGCTCAAATTTAAATAGAAATTTACAGCTATTT | |
| | GAATGGTCAGATATACCAAGAAAGAAAAATATT | |
| | TCTGTTCCTCAAGAAAACTTGCTACCCTCTGTGAGGG | |
| | GAATTTTGCTAAACTTGACATCTTTATAACATG | |
| | AGCCAGATTGAAAGGGAGTGATTTTCATTCATCTTAG | |
| | GTCATGTTATTTCATATTTGTTTCTGAAGGTGC | |
| | GATAGCTCTGTTTTAGGTTTTGCTTGCGCCTGTTAATT | |
| | ACTGGAACACCTTATTTTTCATTAAAGGCTTT | |
| | GAAAGCCAATTCTCAAAAATTCAAAAGTGCAAATTA | |
| | ACAGAACAAAAGGAAATCCAGTAGCAACTGCAGT | |
| | CAAGCGAGGGAGTTGACAAGATAAACCTTACGTCCA | |
| | TTCAAGTTATATGCTGGCCTATGAGAGATGAGAG | |
| | TTGGGTCGTTTGTTCTCTTTGTTGATGATTTTAAAAAA | |
| | ACCCTCTAGAATACACATAATAACATAATGAA | |
| | AGCCATATCTCCATGATATATATGTGCACATATATAT | |
| | ACATACATGTGCATGTATGTATCATATTAAGGA | |
| | CCCATGGTACTCTTAAAACACTGTAGAACTCTGTGAC | |
| | GCAGTAAGGAAGGGGCAGATTTGTACAAAAACT | |
| | TTTCTAGATTCCATCAGCAAAAACCAACACAGGTTTG | |
| | TCACGCTGCATGTCTGGCCAGCTAATCTCGGGG | |
| | GAAAAGCTACAAGTTATTTATTTTATTTTAAGAGAAT | |
| | AAAGTAGGTAATAATTTAAGGGATCAAATTCAA | |
| | GGAGGAATGTGCAATTTTAGAGCAAAGATTTGTTTA | |
| | AGGCAAATGAGACTTTGGGAGCATCCCATTCCAG | |
| | TTTTGTCTTTTTTTTCTCTGAAAGAAAAAAGCAAAAA | |
| | ATAAAATAAAATTCCACTTATACCTTCTGACAA | |
| | GTCCCTAAAGGTCTTGAAATAAAAGGTTCTATGCAA | |
| | GTGCAAAGTTTTATAGTTATTTTTATTGCTGATT | |
| | ATTACTATTACTATCTCTGTTGTCTTAAGAGTATGTGC | |
| | TGATTTCAGAGACATCTCAAATTGAAAGAATA | |
| | TCAGATTGCTTTTAAAGTAGCTGAACGAGCCACAGA | |
| | ATATCTGAAATTATTCATTGTTGTTCCTCCACCA | |
| | CCCCCTTTCTCATGGTCTGATTTTTAGAAGAGTGGCA | |
| | TCCTCGTTCTAAAATGTAATGATCACCAAATAC | |
| | GGCCTTCCATCAAATTTGTGAAAACTACAACAGTATA | |
| | ACAGTGACAAACCTAATTCTCTAGCCCAAACCT | |
| | GGTCTGACAATCATTTCCATTTAGAAGTCATTGAATA | |
| | GTTTTCCAAACACTTTCCATGTGTGTTAGCAAA | |
| | TTATTCCTATTTTGTGTAGATGAGGACGTTGAGACTC | |
| | AGAGACATTCAGAGGCACGCTAGAGGTCTCCAG | |
| | CCTAGCTTCCAGCACCATTGGGACTGAATCCAAGTAC | |
| | TCTCACTCTGAACTTCGTGGTTCTGTCCACTAG | |
| | AGACTCTAATATGCAAACAAGCAGTTCAGGAAAGAA | |
| | AGCATGCTAACACATTCATGAAGCAGTATATGAA | |
| | GTTAGAAGAACAAAAGGAAATACAGGAGATGACAA | |
| | GCAACTGAGATATTGTGATATATAATCATGCTCTT | |
| | AGCTTCAGCTAAATTCAGCTAAATTCTTGTACACTGA | |
| | ACCAATGTCATAATCAGGCTTATTTAGAAAACA | |
| | CTTTGAACTATGCTATAAAAGATTATATCAGAATTCA | |
| | TATTATACATGTGTTCACATCAGCGCTACCTGT | |
| | GATGTTTTCATGTATTTATGTATGTGTTATAAATACTT | |
| | GATTTATACATATACAAATGCACATACGTAGT | |
| | GTGTTTGTGTGTTTATGTATAAATTTATAGGCACACA | |
| | ATAATAGAGGTAATTATAAGTAGGATGCGGTAT | |
| | GAATAATTTGCTTAAAATATGCTAAATAACCAAAACT | |
| | GTTTAACGTCATGTTGCTGTTAGTGCTTCCATA | |
| | CTCCACGTGGGTAGGACTACATCACACTTTTCAACTC | |
| | TGTGCAGTACTGCATGGGTGGAAGACATATTTA | |
| | AGATAATGTGCTTCCCAAAACAACTGAATAAAAGCC | |
| | ATCCCACTACATTGAGTGCTTTCTCTGGCTCCTT | |
| | GCAAAGAAAGATACTTTTTGTAATGGTCCAGGAAAG | |
| | GAACATTGCTTTCTTTTTGTCTTTCAGCACATTT | |
| | GTATTATGCTCACCTTGTCTCTGTCTCACTGTGACCCC | |
| | TTTACACTTGAGTTCAGAGTTCAAGCATTCCA | |
| | AATATAAATTGGAATGTTGGCAGCCCAGTGGCTTGA | |
| | AGGCCAATGATGAGCAGTCCAAGACCCCACAGCG | |
| | AGATGAGCAACTCTTAGGAATTCCCACATCCTAGAGT | |
| | GAATGCACCAACTAACAGTATAGAATGCTGTCC | |
| | TTTTCAAAGCGTCCTAACAGCAGGATTACCTGGTCAA | |
| | GTATGGACTTTCTTTGAATCTTTCTTTTCACAA | |
| | ATTGGACTGCCTGTAATACCAATAACATTGTTGTATC | |
| | TAACTAAATAAATGACTGCATATACACACATAC | |
| | CCTCAATTCTCTTGCTTCCCCATTTTCTTTTTCATCCC | |
| | CTGTCTCAGGACTTTTATTTTCAATGTTGACC | |
| | TTTGGTTTGGCCATATATCACTGTTATAGGAAATCTC | |
| | ATGAGAGGAATGGCTAGTGACCCAACTCTCCAA | |
| | ATGTCTAAGTTAGTAGTTACAGCTGATTTTTTATGAT | |
| | GCATAATTGGAATGTGGAGCCTCTGAGGTTGTG | |
| | ATAGCTTGTACATGAATTTCAAATGTCATTCTAAAGA | |
| | ATGAGGGGTGGGAGGGATTTATAGTTAGAAACG | |
| | ACAGTGCAGGAAGGGGTATTTTCTTGTTGTCAGGGCT | |
| | GGAATGAATCACTGCTGCTCAAGTCAAAGGTTC | |
| | TTGAATATCCTTAGTTTTTGCATTTCCCCTCCTTTTCC | |
| | TTTGACCTTTATTTATTTAATTATGTATTTAT | |
| | TTATTTATTTATATACTTTTGCTCCATTCAGCACAAAC | |
| | ACAAAGCAAAGCAAAAAAAAAAATATATATAT | |
| | ATATATCTGTATATGTGTTGTAGGCAAAACACTGTGA | |
| | ATTTCACAACAACCACCACCAAGCAACTATTTT | |
| | GCCATCTTAACATACATCTCAGGAGACGAAATGAGA | |
| | AAAGATGGGGATGTCATTTTTTAGTCTATGCGTT | |
| | TGAGGCCAGGTCCATGTTTATTTATTTCTTTAGTCTAT | |
| | GCATTAATGAAAATGATCCTGAGTGGAGGTTA | |
| | GCTGAACGTTCAATGTACTGGAGCAAGCATCATAAA | |
| | AGCTGCTAGTAGCCATGTGTTTGAACAGGAAAAA | |
| | TATTACAGAAAATGAAATGTAAAGGCCTATATCTTGC | |
| | AGCTTGTATATCTTACTATTGCTTAAAAAATGT | |
| | ATAAAGCAGCTGGAAATGTTTTAAATACAAGGTCTTT | |
| | GAATTAAATGTGGATTTTAAATATGTAATCCCT | |
| | TGACAAATGACCAAATTATGGTGAACTATTGCTCCCT | |
| | GCGTTCTTTGATCATTACCTATGACTTACAAAT | |
| | CTGCCTGGAGATGTGGACATTCTGCATTTGCTTCTGT | |
| | ATCTGGAGAGATGTTTGTATATATCCAGGCCGT | |
| | ATACACACACATTTCCATATCTCTCTACAGATATATT | |
| | TCCCCTTCAATCGTGACCTGGTATTTGGAACTC | |
| | TCCTTTTCATTTGGCTTATCTTCCTTTTAATGTGATGT | |
| | CTCTGTGCTAATACTTACCAGTTCTTGTTTTG | |
| | CAATCTGTTTTGAGGTCCATTGCTTTACTAAGACCCA | |
| | CTGCATCTTGGCTGATTTCAAAGTGACACCTGA | |
| | ATACAGTGTTTAAAAAAAAAAAAGTTTTGTTTGTAAA | |
| | TCATGTGACCAGCTTCTCTCAACCTGACATGGA | |
| | AAGTCTCTTGTACTACAGTGTATTTAATAAAAATGAT | |
| | GTCTTACAATAAATAACATACTCCAAAAGAGAG | |
| | ACTAAAAATGAAAAAAAAAAAAAAAAAAAAAAAAA | |
| SEQ ID NO: 9 | MSSWIRWHGPAMARLWGFCWLVVGFWRAAFACPTSC | NTRK2 full |
| | KCSASRIWCSDPSPGIVAFPRLEPNSVDPENITEIFIANQK | amino acid |
| | RLEIINEDDVEAYVGLRNLTIVDSGLKFVAHKAFLKNSN | |
| | LQHINFTRNKLTSLSRKHFRHLDLSELILVGNPFTCSCDI | |
| | MWIKTLQEAKSSPDTQDLYCLNESSKNIPLANLQIPNCG | |
| | LPSANLAAPNLTVEEGKSITLSCSVAGDPVPNMYWDVG | |
| | NLVSKHMNETSHTQGSLRITNISSDDSGKQISCVAENLV | |
| | GEDQDSVNLTVHFAPTITFLESPTSDHHWCIPFTVKGNP | |
| | KPALQWFYNGAILNESKYICTKIHVTNHTEYHGCLQLD | |
| | NPTHMNNGDYTLIAKNEYGKDEKQISAHFMGWPGIDD | |
| | GANPNYPDVIYEDYGTAANDIGDTTNRSNEIPSTDVTD | |
| | KTGREHLSVYAVVVIASVVGFCLLVMLFLLKLARHSKF | |
| | GMKDFSWFGFGKVKSRQGVGPASVISNDDDSASPLHHI | |
| | SNGSNTPSSSEGGPDAVIIGMTKIPVIENPQYFGITNSQL | |
| | KPDTFVQHIKRHNIVLKRELGEGAFGKVFLAECYNLCP | |
| | EQDKILVAVKTLKDASDNARKDFHREAELLTNLQHEHI | |
| | VKFYGVCVEGDPLIMVFEYMKHGDLNKFLRAHGPDAV | |
| | LMAEGNPPTELTQSQMLHIAQQIAAGMVYLASQHFVH | |
| | RDLATRNCLVGENLLVKIGDFGMSRDVYSTDYYRVGG | |
| | HTMLPIRWMPPESIMYRKFTTESDVWSLGVVLWEIFTY | |
| | GKQPWYQLSNNEVIECITQGRVLQRPRTCPQEVYELML | |
| | GCWQREPHMRKNIKGIHTLLQNLAKASPVYLDILG | |
| SEQ ID NO: 10 | ATTGTTCTGAAAAGGGAGCTAGGCGAAGGAGCCTTT | NTRK2 kinase |
| | GGAAAAGTGTTCCTAGCTGAATGCTATAACCTCTGTC | domain mRNA |
| | CTGAGCAGGACAAGATCTTGGTGGCAGTGAAGACCC | |
| | TGAAGGATGCCAGTGACAATGCACGCAAGGACTTCC | |
| | ACCGTGAGGCCGAGCTCCTGACCAACCTCCAGCATG | |
| | AGCACATCGTCAAGTTCTATGGCGTCTGCGTGGAGG | |
| | GCGACCCCCTCATCATGGTCTTTGAGTACATGAAGCA | |
| | TGGGGACCTCAACAAGTTCCTCAGGGCACACGGCCC | |
| | TGATGCCGTGCTGATGGCTGAGGGCAACCCGCCCAC | |
| | GGAACTGACGCAGTCGCAGATGCTGCATATAGCCCA | |
| | GCAGATCGCCGCGGGCATGGTCTACCTGGCGTCCCA | |
| | GCACTTCGTGCACCGCGATTTGGCCACCAGGAACTGC | |
| | CTGGTCGGGGAGAACTTGCTGGTGAAAATCGGGGAC | |
| | TTTGGGATGTCCCGGGACGTGTACAGCACTGACTACT | |
| | ACAGGGTCGGTGGCCACACAATGCTGCCCATTCGCT | |
| | GGATGCCTCCAGAGAGCATCATGTACAGGAAATTCA | |
| | CGACGGAAAGCGACGTCTGGAGCCTGGGGGTCGTGT | |
| | TGTGGGAGATTTTCACCTATGGCAAACAGCCCTGGTA | |
| | CCAGCTGTCAAACAATGAGGTGATAGAGTGTATCAC | |
| | TCAGGGCCGAGTCCTGCAGCGACCCCGCACGTGCCC | |
| | CCAGGAGGTGTATGAGCTGATGCTGGGGTGCTGGCA | |
| | GCGAGAGCCCCACATGAGGAAGAACATCAAGGGCAT | |
| | CCATACCCTCCTT | |
| SEQ ID NO: 11 | IVLKRELGEGAFGKVFLAECYNLCPEQDKILVAVKTLK | NTRK2 kinase |
| | DASDNARKDFHREAELLTNLQHEHIVKFYGVCVEGDPL | domain amino |
| | IMVFEYMKHGDLNKFLRAHGPDAVLMAEGNPPTELTQ | acid |
| | SQMLHIAQQIAAGMVYLASQHFVHRDLATRNCLVGEN | |
| | LLVKIGDFGMSRDVYSTDYYRVGGHTMLPIRWMPPESI | |
| | MYRKFTTESDVWSLGVVLWEIFTYGKQPWYQLSNNEV | |
| | IECITQGRVLQRPRTCPQEVYELMLGCWQREPHMRKNI | |
| | KGIHTLL | |
| SEQ ID NO: 15 | ACATTTCTGCAGCCGCGCGGCGAGCCATTCGCGGCG | NTRK3 full |
| | GCTGCTGCAGCTCCTACTGCATCTTCCTTCTCTT | mRNA |
| | CCTTTCCTCGGGCTCCGGTCTCGGAGTCGGAGAGCGC | |
| | GCCTCGCTTCCAGAGCCCCCGGACCCGGCGAGT | |
| | CAGCGATCGCCGAGCCGGCCACCATGCCCGGCAGAC | |
| | CGCGCCACTAGGCGCTCCTCGCGGCTCCCACCCG | |
| | GCGGCGGCGGCGGCGGCGGCGGCGTCCGCGATGGTT | |
| | TCAGACGCTGAAGGATTTTGCATCTGATCGCTCG | |
| | GCGTTTCAAAGAAGCAGCGATCGGAGATGGATGTCT | |
| | CTCTTTGCCCAGCCAAGTGTAGTTTCTGGCGGAT | |
| | TTTCTTGCTGGGAAGCGTCTGGCTGGACTATGTGGGC | |
| | TCCGTGCTGGCTTGCCCTGCAAATTGTGTCTGC | |
| | AGCAAGACTGAGATCAATTGCCGGCGGCCGGACGAT | |
| | GGGAACCTCTTCCCCCTCCTGGAAGGGCAGGATT | |
| | CAGGGAACAGCAATGGGAACGCCAGTATCAACATCA | |
| | CGGACATCTCAAGGAATATCACTTCCATACACAT | |
| | AGAGAACTGGCGCAGTCTTCACACGCTCAACGCCGT | |
| | GGACATGGAGCTCTACACCGGACTTCAAAAGCTG | |
| | ACCATCAAGAACTCAGGACTTCGGAGCATTCAGCCC | |
| | AGAGCCTTTGCCAAGAACCCCCATTTGCGTTATA | |
| | TAAACCTGTCAAGTAACCGGCTCACCACACTCTCGTG | |
| | GCAGCTCTTCCAGACGCTGAGTCTTCGGGAATT | |
| | GCAGTTGGAGCAGAACTTTTTCAACTGCAGCTGTGAC | |
| | ATCCGCTGGATGCAGCTCTGGCAGGAGCAGGGG | |
| | GAGGCCAAGCTCAACAGCCAGAACCTCTACTGCATC | |
| | AACGCTGATGGCTCCCAGCTTCCTCTCTTCCGCA | |
| | TGAACATCAGTCAGTGTGACCTTCCTGAGATCAGCGT | |
| | GAGCCACGTCAACCTGACCGTACGAGAGGGTGA | |
| | CAATGCTGTTATCACTTGCAATGGCTCTGGATCACCC | |
| | CTTCCTGATGTGGACTGGATAGTCACTGGGCTG | |
| | CAGTCCATCAACACTCACCAGACCAATCTGAACTGG | |
| | ACCAATGTTCATGCCATCAACTTGACGCTGGTGA | |
| | ATGTGACGAGTGAGGACAATGGCTTCACCCTGACGT | |
| | GCATTGCAGAGAACGTGGTGGGCATGAGCAATGC | |
| | CAGTGTTGCCCTCACTGTCTACTATCCCCCACGTGTG | |
| | GTGAGCCTGGAGGAGCCTGAGCTGCGCCTGGAG | |
| | CACTGCATCGAGTTTGTGGTGCGTGGCAACCCCCCAC | |
| | CAACGCTGCACTGGCTGCACAATGGGCAGCCTC | |
| | TGCGGGAGTCCAAGATCATCCATGTGGAATACTACC | |
| | AAGAGGGAGAGATTTCCGAGGGCTGCCTGCTCTT | |
| | CAACAAGCCCACCCACTACAACAATGGCAACTATAC | |
| | CCTCATTGCCAAAAACCCACTGGGCACAGCCAAC | |
| | CAGACCATCAATGGCCACTTCCTCAAGGAGCCCTTTC | |
| | CAGAGAGCACGGATAACTTTATCTTGTTTGACG | |
| | AAGTGAGTCCCACACCTCCTATCACTGTGACCCACAA | |
| | ACCAGAAGAAGACACTTTTGGGGTATCCATAGC | |
| | AGTTGGACTTGCTGCTTTTGCCTGTGTCCTGTTGGTG | |
| | GTTCTCTTCGTCATGATCAACAAATATGGTCGA | |
| | CGGTCCAAATTTGGAATGAAGGGTCCCGTGGCTGTC | |
| | ATCAGTGGTGAGGAGGACTCAGCCAGCCCACTGC | |
| | ACCACATCAACCACGGCATCACCACGCCCTCGTCACT | |
| | GGATGCCGGGCCCGACACTGTGGTCATTGGCAT | |
| | GACTCGCATCCCTGTCATTGAGAACCCCCAGTACTTC | |
| | CGTCAGGGACACAACTGCCACAAGCCGGACACG | |
| | TATGTGCAGCACATTAAGAGGAGAGACATCGTGCTG | |
| | AAGCGAGAACTGGGTGAGGGAGCCTTTGGAAAGGTC | |
| | TTCCTGGCCGAGTGCTACAACCTCAGCCCGACCAAG | |
| | GACAAGATGCTTGTGGCTGTGAAGGCCCTGAA | |
| | GGATCCCACCCTGGCTGCCCGGAAGGATTTCCAGAG | |
| | GGAGGCCGAGCTGCTCACCAACCTGCAGCATGAG | |
| | CACATTGTCAAGTTCTATGGAGTGTGCGGCGATGGG | |
| | GACCCCCTCATCATGGTCTTTGAATACATGAAGC | |
| | ATGGAGACCTGAATAAGTTCCTCAGGGCCCATGGGC | |
| | CAGATGCAATGATCCTTGTGGATGGACAGCCACG | |
| | CCAGGCCAAGGGTGAGCTGGGGCTCTCCCAAATGCT | |
| | CCACATTGCCAGTCAGATCGCCTCGGGTATGGTG | |
| | TACCTGGCCTCCCAGCACTTTGTGCACCGAGACCTGG | |
| | CCACCAGGAACTGCCTGGTTGGAGCGAATCTGC | |
| | TAGTGAAGATTGGGGACTTCGGCATGTCCAGAGATG | |
| | TCTACAGCACGGATTATTACAGGCTCTTTAATCC | |
| | ATCTGGAAATGATTTTTGTATATGGTGTGAGGTGGGA | |
| | GGACACACCATGCTCCCCATTCGCTGGATGCCT | |
| | CCTGAAAGCATCATGTACCGGAAGTTCACTACAGAG | |
| | AGTGATGTATGGAGCTTCGGGGTGATCCTCTGGG | |
| | AGATCTTCACCTATGGAAAGCAGCCATGGTTCCAACT | |
| | CTCAAACACGGAGGTCATTGAGTGCATTACCCA | |
| | AGGTCGTGTTTTGGAGCGGCCCCGAGTCTGCCCCAAA | |
| | GAGGTGTACGATGTCATGCTGGGGTGCTGGCAG | |
| | AGGGAACCACAGCAGCGGTTGAACATCAAGGAGATC | |
| | TACAAAATCCTCCATGCTTTGGGGAAGGCCACCC | |
| | CAATCTACCTGGACATTCTTGGCTAGTGGTGGCTGGT | |
| | GGTCATGAATTCATACTCTGTTGCCTCCTCTCT | |
| | CCCTGCCTCACATCTCCCTTCCACCTCACAACTCCTTC | |
| | CATCCTTGACTGAAGCGAACATCTTCATATAA | |
| | ACTCAAGTGCCTGCTACACATACAACACTGAAAAAA | |
| | GGAAAAAAAAAGAAAGAAAAAAAAACCC | |
| SEQ ID NO: 16 | MDVSLCPAKCSFWRIFLLGSVWLDYVGSVLACPANCV | NTRK3 full |
| | CSKTEINCRRPDDGNLFPLLEGQDSGNSNGNASINITDIS | amino acid |
| | RNITSIHIENWRSLHTLNAVDMELYTGLQKLTIKNSGLR | |
| | SIQPRAFAKNPHLRYINLSSNRLTTLSWQLFQTLSLREL | |
| | QLEQNFFNCSCDIRWMQLWQEQGEAKLNSQNLYCINA | |
| | DGSQLPLFRMNISQCDLPEISVSHVNLTVREGDNAVITC | |
| | NGSGSPLPDVDWIVTGLQSINTHQTNLNWTNVHAINLT | |
| | LVNVTSEDNGFTLTCIAENVVGMSNASVALTVYYPPRV | |
| | VSLEEPELRLEHCIEFVVRGNPPPTLHWLHNGQPLRESK | |
| | IIHVEYYQEGEISEGCLLFNKPTHYNNGNYTLIAKNPLG | |
| | TANQTINGHFLKEPFPESTDNFILFDEVSPTPPITVTHKPE | |
| | EDTFGVSIAVGLAAFACVLLVVLFVMINKYGRRSKFGM | |
| | KGPVAVISGEEDSASPLHHINHGITTPSSLDAGPDTVVIG | |
| | MTRIPVIENPQYFRQGHNCHKPDTYVQHIKRRDIVLKRE | |
| | LGEGAFGKVFLAECYNLSPTKDKMLVAVKALKDPTLA | |
| | ARKDFQREAELLTNLQHEHIVKFYGVCGDGDPLIMVFE | |
| | YMKHGDLNKFLRAHGPDAMILVDGQPRQAKGELGLSQ | |
| | MLHIASQIASGMVYLASQHFVHRDLATRNCLVGANLL | |
| | VKIGDFGMSRDVYSTDYYRLFNPSGNDFCIWCEVGGHT | |
| | MLPIRWMPPESIMYRKFTTESDVWSFGVILWEIFTYGK | |
| | QPWFQLSNTEVIECITQGRVLERPRVCPKEVYDVMLGC | |
| | WQREPQQRLNIKEIYKILHALGKATPIYLDILG | |
| SEQ ID NO: 17 | ATCGTGCTGAAGCGAGAACTGGGTGAGGGAGCCTTT | NTRK3 kinase |
| | GGAAAGGTCTTCCTGGCCGAGTGCTACAACCTCAGC | domain mRNA |
| | CCGACCAAGGACAAGATGCTTGTGGCTGTGAAGGCC | |
| | CTGAAGGATCCCACCCTGGCTGCCCGGAAGGATTTCC | |
| | AGAGGGAGGCCGAGCTGCTCACCAACCTGCAGCATG | |
| | AGCACATTGTCAAGTTCTATGGAGTGTGCGGCGATG | |
| | GGGACCCCCTCATCATGGTCTTTGAATACATGAAGCA | |
| | TGGAGACCTGAATAAGTTCCTCAGGGCCCATGGGCC | |
| | AGATGCAATGATCCTTGTGGATGGACAGCCACGCCA | |
| | GGCCAAGGGTGAGCTGGGGCTCTCCCAAATGCTCCA | |
| | CATTGCCAGTCAGATCGCCTCGGGTATGGTGTACCTG | |
| | GCCTCCCAGCACTTTGTGCACCGAGACCTGGCCACCA | |
| | GGAACTGCCTGGTTGGAGCGAATCTGCTAGTGAAGA | |
| | TTGGGGACTTCGGCATGTCCAGAGATGTCTACAGCAC | |
| | GGATTATTACAGGCTCTTTAATCCATCTGGAAATGAT | |
| | TTTTGTATATGGTGTGAGGTGGGAGGACACACCATGC | |
| | TCCCCATTCGCTGGATGCCTCCTGAAAGCATCATGTA | |
| | CCGGAAGTTCACTACAGAGAGTGATGTATGGAGCTT | |
| | CGGGGTGATCCTCTGGGAGATCTTCACCTATGGAAA | |
| | GCAGCCATGGTTCCAACTCTCAAACACGGAGGTCATT | |
| | GAGTGCATTACCCAAGGTCGTGTTTTGGAGCGGCCCC | |
| | GAGTCTGCCCCAAAGAGGTGTACGATGTCATGCTGG | |
| | GGTGCTGGCAGAGGGAACCACAGCAGCGGTTGAACA | |
| | TCAAGGAGATCTACAAAATCCTC | |
| SEQ ID NO: 18 | IVLKRELGEGAFGKVFLAECYNLSPTKDKMLVAVKAL | NTRK3 kinase |
| | KDPTLAARKDFQREAELLTNLQHEHIVKFYGVCGDGDP | domain amino |
| | LIMVFEYMKHGDLNKFLRAHGPDAMILVDGQPRQAKG | acid |
| | ELGLSQMLHIASQIASGMVYLASQHFVHRDLATRNCLV | |
| | GANLLVKIGDFGMSRDVYSTDYYRLFNPSGNDFCIWCE | |
| | VGGHTMLPIRWMPPESIMYRKFTTESDVWSFGVILWEI | |
| | FTYGKQPWFQLSNTEVIECITQGRVLERPRVCPKEVYD | |
| | VMLGCWQREPQQRLNIKEIYKIL | |

Normally, physiological activation of the receptor through ligand binding activates the kinase domain, leading to receptor homodimerization, phosphorylation and activation of downstream signaling pathways. Although highly homologous, each receptor has a preferred ligand: TRKA has the highest affinity for neurotrophin nerve growth factor, TRKB has the highest affinity for brain-derived neurotrophic factor and neurotrophin-4 and TRKC has the highest affinity for neurotrophin-3. See, e.g., Solomon et al., Annals of Oncology, 30 (Suppl 8): viii 16 (2019).

Constitutive activation of the NTRKs and subsequent downstream pathways can occur through an in-frame fusion of the C-terminal tyrosine kinase domain of any of the NTRK genes with a fusion partner. A multitude of fusion partners have been described, and in virtually all cases, the fusion eliminates the ligand binding site, resulting in ligand-independent dimerization and phosphorylation. See, e.g., Solomon et al., Annals of Oncology, 30 (Suppl 8): viii 16 (2019).

Common NTRK gene fusion products include, but not limited to, NTRK1 fusion genes such as TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, and MRPL24-NTRK1; NTRK2 fusion genes such as OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, and SQSTM1-NTRK2; and NTRK3 fusion genes such as ETV6-NTRK3, BTBD1-NTRK3, EML4-NTRK3, TFG-NTRK3, RBPMS-NTRK3, LYN-NTRK3, and NTRK3-HOMER2.

In some embodiments, the method of detecting a NTRK fusion gene disclosed herein comprises detection of NTRK1 fusion gene. In some embodiments, NTRK1 fusion gene is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, and MRPL24-NTRK1.

In some embodiments, the method of detecting a NTRK fusion gene disclosed herein comprises detection of NTRK2 fusion gene. In some embodiments, the NTRK2 fusion gene is selected from a group consisting of OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, and SQSTM1-NTRK2

In some embodiments, the method of detecting a NTRK fusion gene disclosed herein comprises detection of NTRK3 fusion gene. In some embodiments, the NTRK3 fusion gene is selected from a group consisting of ETV6-NTRK3, BTBD1-NTRK3, EML4-NTRK3, TFG-NTRK3, RBPMS-NTRK3, LYN-NTRK3, and NTRK3-HOMER2.

### 7.3. Methods of Detecting NTRK Gene Fusion

In one aspect, provided herein is a method of detecting NTRK fusion using RNA in situ hybridization (ISH).

There are currently four main testing methodologies for NTRK gene fusions including IHC, DNA Fluorescence In Situ Hybridization (FISH), RT-PCR, and next generation sequencing (NGS).

IHC is a technique of visualizing proteins in histologic tissue sections using antibodies that recognize specific proteins and secondary chromogenic detection reagents. Pan-TRK IHC utilizes antibodies that detect all 3 fusion proteins—TRKA, TRKB, and TRKC—in addition to wild-type TRK proteins. Pan-TRK IHC detects expression of the TRK proteins, whose expression levels are potentially driven by the NTRK gene fusions. However, the technique lacks specificity to be a stand-alone assay, as it does not confirm the presence of a gene fusion event. Confirmatory testing with another technique such as NGS should be performed on Pan-TRK IHC positive tumors. In addition, for a subset of cases, in particular those with smooth muscle and neuronal differentiation, the interpretation of IHC results may prove challenging in tissues where TRKs are physiologically expressed. Last but not least, IHC has much weaker staining in tumors harboring NTRK3 fusions, a relatively more abundant gene fusion type, than tumors with NTRK1/NTRK2 fusions, which will significantly affect its sensitivity. In other words, pan-TRK IHC staining, particularly for low or weak expression, can appear negative or very weak in appearance (cytoplasmic staining), with signal difficult to distinguish over background. As such, IHC can lead to false negatives in cases of weak expression, and thereby disqualify patients from potentially life-saving therapy.

FISH is a DNA-based technique that allows for the detection of translocations, amplifications, or deletions on intact chromosomes. Fluorescence associated with so-called break-apart probes allows visualization of gene fusion events in the histologic context of the tumor. For NTRK gene fusions, three sets of break-apart probes are required, one set for each NTRK gene. In addition, it is typically a time and labor intensive manual procedure; lab automation for FISH, while available on some instruments, is not commonly used in anatomic pathology laboratories.

RT-PCR is a targeted amplification of a predefined region of interest on an RNA template. RT-PCR for NTRK gene fusions can detect fusion transcripts with known partners. One of the shortcomings of the technique is that it can be difficult to amplify across large introns and to identify NTRK fusions involving unknown or novel fusion partners. In addition, it cannot be performed using lab automation common in anatomic pathology laboratories across the US.

NGS is a high throughput technology that is capable of sequencing large numbers of DNA molecules in a massively parallel fashion. However, NGS has a relatively long turnaround time (1-3 weeks, compared to same-day results by IHC), and this longer turnaround time may represent a major issue in practice, and also hinders NGS serving as a screening tool. Additionally, NGS cannot be performed in typical pathology labs, instead it requires specialized instruments, facilities, and personnel, which significantly limit the wide usage of the technology, especially as a screening tool. Moreover, NGS can be expensive, which also limits its usage as a front-line detection method.

Detection methods using RNA in situ hybridization, as described herein, solves the above-mentioned deficiencies of currently available approaches and provides additional advantages. For example, detection methods of the present disclosure can be performed using lab automation common in anatomic pathology laboratories, making the use of these methods widely accessible and practical for testing larger numbers of tumors. Other techniques such as NGS, PCR, and DNA FISH are not as widely available or cost-efficient.

Additionally, the detection methods provided herein support accurate and straight forward interpretation of results, with clear-cut chromogenic dot signals indicating positive RNA expression, for example. In many cases, the signals are easier to interpret as compared to immunohistochemistry staining due to the clear identification of positive vs. negative signals. Unlike the sometimes weak signals from IHC, the chromogenic dots resulting from detection of, for example, NTRK RNA in the context of the methods described herein can be more readily identified with minimal background interference. An additional advantage of the detection methods of the present disclosure is that it is highly specific and includes specific probes for NTRK1, NTRK2, and NTRK3, unlike the IHC approach which uses an antibody that recognizes all three proteins.

Finally, the method provided herein does not require knowledge of the fusion partner for NTRK gene fusions. Because all the NTRK genes are upregulated as a result of the gene fusion events, any NTRK gene fusion can potentially be detected with this approach, regardless of the fusion partner. This is an advantage over some other techniques such as DNA FISH and RT-PCR, which require knowledge of the fusion partner.

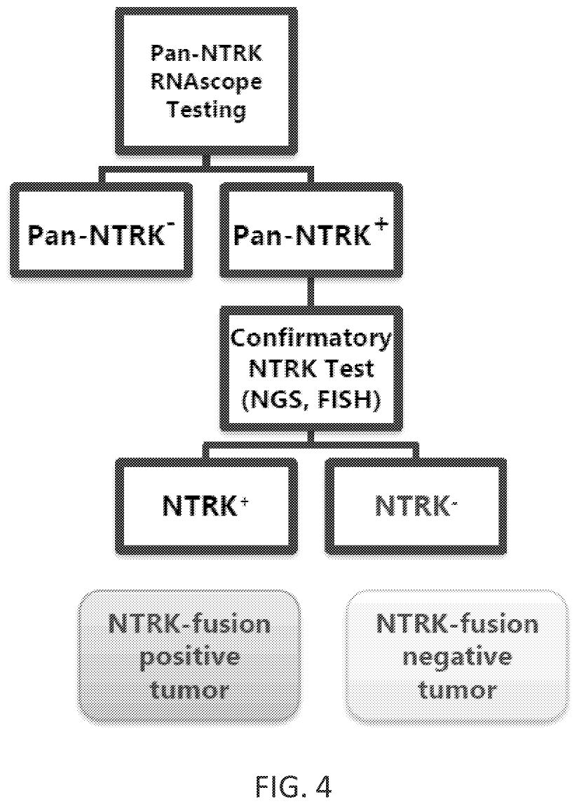

In accordance with these advantages, embodiments of the present disclosure include methods for detecting NTRK gene fusions using the RNAscope® detection platform. In one embodiment of the present disclosure, the NTRK gene fusion detection methods include use of an algorithm or workflow, as represented schematically in FIG. 4. For example, the algorithm or workflow can be implemented using an automated platform (e.g., standard protocol on Bond III clinical platform), and reproducible results can be obtained without necessarily needing to optimize a protocol. In some embodiments, the algorithm or workflow is designed to implement chromogenic detection methods using standard light microscope analysis and evaluation, which is typically used by pathologists in a clinical setting. In some embodiments, the algorithm or workflow implements the detection methods described further herein (e.g., for NTRK gene fusions), including the acquisition of test results (e.g., data from patient samples) and the accurate interpretation of the results. These methods facilitate the acquisition of results with high signal-to-noise ratios, a high degree of reproducibility, and high sensitivity for positives and clear-cut negatives (e.g., high negative predictive value or NPV). In some embodiments, the algorithm or workflow includes a confirmatory test as part of the methods (e.g., using NGS or FISH), and/or the ability to detect additional makers in a given sample from a patient (e.g., multiplexing).

The methods provided herein generally relate to RNA in situ detection of target nucleic acids (i.e., a NTRK fusion). Methods for in situ detection of nucleic acids are well known to those skilled in the art (see, for example, US 2008/0038725; US 2009/0081688; Hicks et al., J. Mol. Histol. 35:595-601 (2004)). As used herein, “in situ hybridization” or “ISH” refers to a type of hybridization that uses a directly or indirectly labeled complementary DNA or RNA strand, such as a probe, to bind to and localize a specific nucleic acid, such as mRNA, in a sample, in particular a portion or section of tissue or cells (in situ). The probe types can be double stranded DNA (dsDNA), single stranded DNA (ssDNA), single stranded complimentary RNA (sscRNA), messenger RNA (mRNA), micro RNA (miRNA), ribosomal RNA, mitochondrial RNA, and/or synthetic oligonucleotides.

In one embodiment, the RNA ISH used herein to detect NTRK fusion is RNAscope®, which is described in more detail in, e.g., U.S. Pat. Nos. 7,709,198, 8,604,182, and 8,951,726. Specifically, RNAscope® describes using specially designed oligonucleotide probes, sometimes referred to as “double-Z” or ZZ probes, in combination with a branched-DNA-like signal amplification system to reliably detect RNA as small as 1 kilobase at single-molecule sensitivity under standard bright-field microscopy (Anderson et al., J. Cell. Biochem. 117(10):2201-2208 (2016); Wang et al., J. Mol. Diagn. 14(1):22-29 (2012)). Such a probe design greatly improves the specificity of signal amplification because only when both probes in each pair bind to their intended target can signal amplification occur.

Thus, in some embodiments, provided herein is a method of detecting a neurotrophic tyrosine receptor kinase (NTRK) fusion gene, comprising: (i) obtaining a sample; (ii) providing at least one set of two or more target probes capable of hybridizing to a target nucleic acid; (iii) providing a SGC capable of hybridizing to said set of two or more target probes, wherein said SGC comprises a label probe and a nucleic acid component capable of hybridizing to said set of two or more target probes; (iv) hybridizing said target nucleic acid to said set of two or more target probes; and (v) capturing the SGC to said set of two or more target probes and thereby capturing the SGC to said target nucleic acid, wherein the target nucleic acid comprises a region of mRNA encoding a kinase domain of NTRK1, NTRK2, or NTRK3.

In some embodiments, the target probes comprises a target (T) section and a label (L) section, wherein the T section is a nucleic acid sequence complementary to a section on the target nucleic acid and the L section is a nucleic acid sequence complementary to a section on the nucleic acid component of the signal generating complex, and wherein the T sections of the two or more target probes are complementary to non-overlapping regions of the target nucleic acid, and the L sections of the two or more target probes are complementary to non-overlapping regions of the nucleic acid component of the generating complex.

In some embodiments, in all the two or more target probes the T section is at 5′ of the L section or in all the two or more target probes the T section is at 3′ of the L section.

In some embodiments, plural sets of target probes (e.g., 2 to 25 sets of target probes) are used to detect NTRK1 fusion, NTRK2 fusion, or NTRK3 fusion.

In some embodiments, one or more sets of target probes are used to detect one of NTRK1 fusion, NTRK2 fusion, and NTRK3 fusion. In other embodiments, two or more sets of target probes are used to detect two of the NTRK1 fusion, NTRK2 fusion, and NTRK3 fusion. In yet other embodiments, at least three sets of target probes are used to detect all of the NTRK1 fusion, NTRK2 fusion, and NTRK3 fusion, which each set of target probes for one of the three types of fusions.

In some embodiments of the various RNA in situ hybridization methods provided herein, method comprises using a probe comprising a nucleic acid sequence complementary to a region of mRNA encoding a kinase domain of NTRK1, NTRK2, or NTRK3. In some embodiments, a probe comprising a nucleic acid sequence complementary to a region of mRNA encoding a kinase domain of NTRK1, NTRK2, or NTRK3 comprises plural probes, each of which comprises a region complementary to a region of the mRNA encoding the kinase domain of NTRK1, NTRK2, or NTRK3. As described above, the mRNA encoding the kinase domain of NTRK1 comprises SEQ ID NO:3, the mRNA encoding the kinase domain of NTRK2 comprises SEQ ID NO:10, and the mRNA encoding the kinase domain of NTRK3 comprises SEQ ID NO:17.

In other embodiments, the method comprises using one or more probes, each of which comprises a region complementary to a region of the mRNA encoding the kinase domain of NTRK1. In other embodiments, the method comprises using one or more probes, each of which comprises a region complementary to a region of the mRNA encoding the kinase domain of NTRK2. In yet other embodiments, the method comprises using one or more probes, each of which comprises a region complementary to a region of the mRNA encoding the kinase domain of NTRK3.

In other embodiments, method comprises detecting a NTRK fusion gene in the sample by RNA in situ hybridization using a probe pool comprising at least two of (a) a first probe complementary to a region of the mRNA encoding the kinase domain of NTRK1; (b) a second probe complementary to a region of the mRNA encoding the kinase domain of NTRK2; and (c) a third probe complementary to a region of the mRNA encoding the kinase domain of NTRK3.

In some embodiments, the probe pool comprises the first probe complementary to a region of the mRNA encoding the kinase domain of NTRK1; and the second probe complementary to a region of the mRNA encoding the kinase domain of NTRK2. In other embodiments, the probe pool comprises the first probe complementary to a region of the mRNA encoding the kinase domain of NTRK1; and the third probe complementary to a region of the mRNA encoding the kinase domain of NTRK3. In yet other embodiments, the probe pool comprises the second probe complementary to a region of the mRNA encoding the kinase domain of NTRK2; and the third probe complementary to a region of the mRNA encoding the kinase domain of NTRK3. In yet other embodiments, the probe pool comprises the first probe complementary to a region of the mRNA encoding the kinase domain of NTRK1; the second probe complementary to a region of the mRNA encoding the kinase domain of NTRK2; and the third probe complementary to a region of the mRNA encoding the kinase domain of NTRK3.

As used herein, the term “a probe comprising a nucleic acid sequence complementary to a region” refers a probe, at least part of which is complementary to the region. Thus, the probe may contain additional nucleic acid sequence that is not complementary to the region. For example, one part of a probe provided herein is complementary to a 3′ prime or 5′ prime region of the mRNA encoding the kinase domain of NTRK1, and the rest of the probe is complementary to a region outside the kinase domain.

In a specific embodiment, the probe that comprises a region complementary to a region of the mRNA encoding the kinase domain of NTRK1, is complementary to a region of SEQ ID NO:5. In some embodiments, the method comprises using two or more probes complementary to different regions within SEQ ID NO:5. In a specific embodiment, the probe that comprises a region complementary to a region of the mRNA encoding the kinase domain of NTRK1, is complementary to a region SEQ ID NO:6. In some embodiments, the method comprises using two or more probes complementary to different regions within SEQ ID NO:6. In a specific embodiment, the probe that comprises a region complementary to a region of the mRNA encoding the kinase domain of NTRK1, is complementary to a region of SEQ ID NO:7. In some embodiments, the method comprises using two or more probes complementary to different regions within SEQ ID NO:7.

In a specific embodiment, the probe that comprises a region complementary to a region of the mRNA encoding the kinase domain of NTRK2, is complementary to a region of SEQ ID NO:12. In some embodiments, the method comprises using two or more probes complementary to different regions within SEQ ID NO:12. In a specific embodiment, the probe that comprises a region complementary to a region of the mRNA encoding the kinase domain of NTRK2, is complementary to a region of SEQ ID NO:13. In some embodiments, the method comprises using two or more probes complementary to different regions within SEQ ID NO:13. In a specific embodiment, the probe that comprises a region complementary to a region of the mRNA encoding the kinase domain of NTRK2, is complementary to a region of SEQ ID NO:14. In some embodiments, the method comprises using two or more probes complementary to different regions within SEQ ID NO:14.

In a specific embodiment, the probe that comprises a region complementary to a region of the mRNA encoding the kinase domain of NTRK3, is complementary to a region of SEQ ID NO:19. In some embodiments, the method comprises using two or more probes complementary to different regions within SEQ ID NO:19. In a specific embodiment, the probe that comprises a region complementary to a region of the mRNA encoding the kinase domain of NTRK3, is complementary to a region of SEQ ID NO:20. In some embodiments, the method comprises using two or more probes complementary to different regions within SEQ ID NO:20. In a specific embodiment, the probe that comprises a region complementary to a region of the mRNA encoding the kinase domain of NTRK3, is complementary to a region of SEQ ID NO:21. In some embodiments, the method comprises using two or more probes complementary to different regions within SEQ ID NO:21.

In some embodiments, the probes used in the RNA in situ hybridization provided herein comprise plural probes complementary to different regions within SEQ ID NO:5, plural probes complementary to different regions within SEQ ID NO:12, and plural probes complementary to different regions within SEQ ID NO:19.

In some embodiments, the probes used in the RNA in situ hybridization provided herein comprise plural probes complementary to different regions within SEQ ID NO:6, plural probes complementary to different regions within SEQ ID NO:13, and plural probes complementary to different regions within SEQ ID NO:20.

In some embodiments, the probes used in the RNA in situ hybridization provided herein comprise plural probes complementary to different regions within SEQ ID NO:7, plural probes complementary to different regions within SEQ ID NO:14, and plural probes complementary to different regions within SEQ ID NO:21.

In some embodiments, the probes used in the RNA in situ hybridization provided herein comprise plural probes complementary to different regions within a sequence that is selected from the group consisting of SEQ ID NO:3, SEQ ID NO:5, SEQ ID NO:6, and SEQ ID NO:7; plural probes complementary to different regions within a sequence that is selected from the group consisting of SEQ ID NO:10, SEQ ID NO:12, SEQ ID NO:13, and SEQ ID NO:14; and plural probes complementary to different regions within a sequence that is selected from the group consisting of SEQ ID NO:17, SEQ ID NO:19, SEQ ID NO:20, and SEQ ID NO:21.

SEQ ID NOs:5-7, 12-14 and 19-21 are as shown in the table below.

| SEQ ID # | Sequence | Name |
|---|---|---|
| SEQ ID NO: 5 | AGGCGTCCGAGAGTGCTCGGCAGGACTTCCAGCGT | NTRK1 region |
| | GAGGCTGAGCTGCTCACCATGCTGCAGCACCAGCA | (mRNA) targeted |
| | CATCGTGCGCTTCTTCGGCGTCTGCACCGAGGGCCG | by ACD Probe No. |
| | CCCCCTGCTCATGGTCTTTGAGTATATGCGGCACGG | 852778. |
| | GGACCTCAACCGCTTCCTCCGATCCCATGGACCTGA | |
| | TGCCAAGCTGCTGGCTGGTGGGGAGGATGTGGCTC | |
| | CAGGCCCCCTGGGTCTGGGGCAGCTGCTGGCCGTG | |
| | GCTAGCCAGGTCGCTGCGGGGATGGTGTACCTGGC | |
| | GGGTCTGCATTTTGTGCACCGGGACCTGGCCACACG | |
| | CAACTGTCTAGTGGGCCAGGGACTGGTGGTCAAGA | |
| | TTGGTGATTTTGGCATGAGCAGGGATATCTACAGCA | |
| | CCGACTATTACCGTGTGGGAGGCCGCACCATGCTGC | |
| | CCATTCGCTGGATGCCGCCCGAGAGCATCCTGTACC | |
| | GTAAGTTCACCACCGAGAGCGACGTGTGGAGCTTC | |
| | GGCGTGGTGCTCTGGGAGATCTTCACCTACGGCAAG | |
| | CAGCCCTGGTACCAGCTCTCCAACACGGAGGCAAT | |
| | CGACTGCATCACGCAGGGACGTGAGTTGGAGCGGC | |
| | CACGTGCCTGCCCACCAGAGGTCTACGCCATCATGC | |
| | GGGGCTGCTGGCAGCGGGAGCCCCAGCAACGCCAC | |
| | AGCATCAAGGATGTGCACGCCCGGCTGCAAGCCCT | |
| | GGCCCAGGCACCTCCTGTCTACCTGGATGTCCTGGG | |
| | CTAGGGGGCCGGCCCAGGGGCTGGGAGTGGTTAGC | |
| | CGGAATACTGGGGCCTGCCCTCAGCATCCCCCATAG | |
| | CTCCCAGCAGCCCCAGGGTGATCTCAAAGTATCTAA | |
| | TTCACCCTCAGCATGTGGGAAGGGACAGGTGGGGG | |
| | CTGGGAGTAGAGGATGTTCCTGCTTCTCTAGGCAAG | |
| | GTCCCGTCATAGCAATTATATTTATT | |
| SEQ ID NO: 6 | GCTCTTCAATGGCTCCGTGCTCAATGAGACCAGCTT | NTRK1 region |
| | CATCTTCACTGAGTTCCTGGAGCCGGCAGCCAATGA | (mRNA) targeted |
| | GACCGTGCGGCACGGGTGTCTGCGCCTCAACCAGC | by ACD Probe No. |
| | CCACCCACGTCAACAACGGCAACTACACGCTGCTG | 852788. |
| | GCTGCCAACCCCTTCGGCCAGGCCTCCGCCTCCATC | |
| | ATGGCTGCCTTCATGGACAACCCTTTCGAGTTCAAC | |
| | CCCGAGGACCCCATCCCTGACACTAACAGCACATCT | |
| | GGAGACCCGGTGGAGAAGAAGGACGAAACACCTTT | |
| | TGGGGTCTCGGTGGCTGTGGGCCTGGCCGTCTTTGC | |
| | CTGCCTCTTCCTTTCTACGCTGCTCCTTGTGCTCAAC | |
| | AAATGTGGACGGAGAAACAAGTTTGGGATCAACCG | |
| | CCCGGCTGTGCTGGCTCCAGAGGATGGGCTGGCCAT | |
| | GTCCCTGCATTTCATGACATTGGGTGGCAGCTCCCT | |
| | GTCCCCCACCGAGGGCAAAGGCTCTGGGCTCCAAG | |
| | GCCACATCATCGAGAACCCACAATACTTCAGTGATG | |
| | CCTGTGTTCACCACATCAAGCGCCGGGACATCGTGC | |
| | TCAAGTGGGAGCTGGGGGAGGGCGCCTTTGGGAAG | |
| | GTCTTCCTTGCTGAGTGCCACAACCTCCTGCCTGAG | |
| | CAGGACAAGATGCTGGTGGCTGTCAAGGCACTGAA | |
| | GGAGGCGTCCGAGAGTGCTCGGCAGGACTTCCAGC | |
| | GTGAGGCTGAGCTGCTCACCATGCTGCAGCACCAG | |
| | CACATCGTGCGCTTCTTCGGCGTCTGCACCGAGGGC | |
| | CGCCCCCTGCTCATGGTCTTTGAGTATATGCGGCAC | |
| | GGGGACCTCAACCGCTTCCTCCGATCCCATGGACCT | |
| | GATGCCAAGCTGCTGGCTGGTGGGGAGGATGTGGC | |
| | TCCAGGCCCCCTGGGTCTGGGGCAGCTGCTGGCCGT | |
| | GGCTAGCCAGGTCGCTGCGGGGATGGTGTACCTGG | |
| | CGGGTCTGCATTTTGTGCACCGGGACCTGGCCACAC | |
| | GCAACTGTCTAGTGGGCCAGGGACTGGTGGTCAAG | |
| | ATTGGTGATTTTGGCATGAGCAGGGATATCTACAGC | |
| | ACCGACTATTACCGTGTGGGAGGCCGCACCATGCTG | |
| | CCCATTCGCTGGATGCCGCCCGAGAGCATCCTGTAC | |
| | CGTAAGTTCACCACCGAGAGCGACGTGTGGAGCTT | |
| | CGGCGTGGTGCTCTGGGAGATCTTCACCTACGGCAA | |
| | GCAGCCCTGGTACCAGCTCTCCAACACGGAGGCAA | |
| | TCGACTGCATCACGCAGGGACGTGAGTTGGAGCGG | |
| | CCACGTGCCTGCCCACCAGAGGTCTACGCCATCATG | |
| | CGGGGCTGCTGGCAGCGGGAGCCCCAGCAACGCCA | |
| | CAGCATCAAGGATGTGCACGCCCGGCTGCAAGCCC | |
| | TGGCCCAGGCACCTCCTGTCTACCTGGATGTCCTGG | |
| | GCTAGGGGGCCGGCCCAGGGGCTGGGAGTGGTTAG | |
| | CCGGAATACTGGGGCCTGCCCTCAGCATCCCCCATA | |
| | GCTCCCAGCAGCCCCAGGGTGATCTCAAAGTATCTA | |
| | ATTCACCCTCAGCATGTGGGAAGGGACAGGTGGGG | |
| | GCTGGGAGTAGAGGATGTTCCTGCTTCTCTAGGCAA | |
| | GGTCCCGTCATAGC | |
| SEQ ID NO: 7 | AACCAGCCCACCCACGTCAACAACGGCAACTACAC | NTRK1 region |
| | GCTGCTGGCTGCCAACCCCTTCGGCCAGGCCTCCGC | (mRNA) targeted |
| | CTCCATCATGGCTGCCTTCATGGACAACCCTTTCGA | by ACD Probe No. |
| | GTTCAACCCCGAGGACCCCATCCCTGACACTAACAG | 852798. |
| | CACATCTGGAGACCCGGTGGAGAAGAAGGACGAAA | |
| | CACCTTTTGGGGTCTCGGTGGCTGTGGGCCTGGCCG | |
| | TCTTTGCCTGCCTCTTCCTTTCTACGCTGCTCCTTGT | |
| | GCTCAACAAATGTGGACGGAGAAACAAGTTTGGGA | |
| | TCAACCGCCCGGCTGTGCTGGCTCCAGAGGATGGG | |
| | CTGGCCATGTCCCTGCATTTCATGACATTGGGTGGC | |
| | AGCTCCCTGTCCCCCACCGAGGGCAAAGGCTCTGG | |
| | GCTCCAAGGCCACATCATCGAGAACCCACAATACTT | |
| | CAGTGATGCCTGTGTTCACCACATCAAGCGCCGGGA | |
| | CATCGTGCTCAAGTGGGAGCTGGGGGAGGGCGCCT | |
| | TTGGGAAGGTCTTCCTTGCTGAGTGCCACAACCTCC | |
| | TGCCTGAGCAGGACAAGATGCTGGTGGCTGTCAAG | |
| | GCACTGAAGGAGGCGTCCGAGAGTGCTCGGCAGGA | |
| | CTTCCAGCGTGAGGCTGAGCTGCTCACCATGCTGCA | |
| | GCACCAGCACATCGTGCGCTTCTTCGGCGTCTGCAC | |
| | CGAGGGCCGCCCCCTGCTCATGGTCTTTGAGTATAT | |
| | GCGGCACGGGGACCTCAACCGCTTCCTCCGATCCCA | |
| | TGGACCTGATGCCAAGCTGCTGGCTGGTGGGGAGG | |
| | ATGTGGCTCCAGGCCCCCTGGGTCTGGGGCAGCTGC | |
| | TGGCCGTGGCTAGCCAGGTCGCTGCGGGGATGGTG | |
| | TACCTGGCGGGTCTGCATTTTGTGCACCGGGACCTG | |
| | GCCACACGCAACTGTCTAGTGGGCCAGGGACTGGT | |
| | GGTCAAGATTGGTGATTTTGGCATGAGCAGGGATAT | |
| | CTACAGCACCGACTATTACCGTGTGGGAGGCCGCA | |
| | CCATGCTGCCCATTCGCTGGATGCCGCCCGAGAGCA | |
| | TCCTGTACCGTAAGTTCACCACCGAGAGCGACGTGT | |
| | GGAGCTTCGGCGTGGTGCTCTGGGAGATCTTCACCT | |
| | ACGGCAAGCAGCCCTGGTACCAGCTCTCCAACACG | |
| | GAGGCAATCGACTGCATCACGCAGGGACGTGAGTT | |
| | GGAGCGGCCACGTGCCTGCCCACCAGAGGTCTACG | |
| | CCATCATGCGGGGCTGCTGGCAGCGGGAGCCCCAG | |
| | CAACGCCACAGCATCAAGGATGTGCACGCCCGGCT | |
| | GCAAGCCCTGGCCCAGGCACCTCCTGTCTACCTGGA | |
| | TGTCCTGGGCTAGGGGGCCGGCCCAGGGGCTGGGA | |
| | GTGGTTAGCCGGAATACTGGGGCCTGCCCTCAGCAT | |
| | CCCCCATAGCTCCCAGCAGCCCCAGGGTGATCTCAA | |
| | AGTATCTAATTCACCCTCAGCATGTGGGAAGGGAC | |
| | AGGTGGGGGCTGGGAGTAGAGGATGTTCCTGCTTCT | |
| | CTAGGCAAGGTCCCGTCATAGC | |
| SEQ ID NO: 12 | AAATTATCCTGATGTAATTTATGAAGATTATGGAAC | NTRK2 region |
| | TGCAGCGAATGACATCGGGGACACCACGAACAGAA | (mRNA) targeted |
| | GTAATGAAATCCCTTCCACAGACGTCACTGATAAAA | by ACD Probe No. |
| | CCGGTCGGGAACATCTCTCGGTCTATGCTGTGGTGG | 852808 |
| | TGATTGCGTCTGTGGTGGGATTTTGCCTTTTGGTAAT | |
| | GCTGTTTCTGCTTAAGTTGGCAAGACACTCCAAGTT | |
| | TGGCATGAAAGATTTCTCATGGTTTGGATTTGGGAA | |
| | AGTAAAATCAAGACAAGGTGTTGGCCCAGCCTCCG | |
| | TTATCAGCAATGATGATGACTCTGCCAGCCCACTCC | |
| | ATCACATCTCCAATGGGAGTAACACTCCATCTTCTT | |
| | CGGAAGGTGGCCCAGATGCTGTCATTATTGGAATG | |
| | ACCAAGATCCCTGTCATTGAAAATCCCCAGTACTTT | |
| | GGCATCACCAACAGTCAGCTCAAGCCAGACACATT | |
| | TGTTCAGCACATCAAGCGACATAACATTGTTCTGAA | |
| | AAGGGAGCTAGGCGAAGGAGCCTTTGGAAAAGTGT | |
| | TCCTAGCTGAATGCTATAACCTCTGTCCTGAGCAGG | |
| | ACAAGATCTTGGTGGCAGTGAAGACCCTGAAGGAT | |
| | GCCAGTGACAATGCACGCAAGGACTTCCACCGTGA | |
| | GGCCGAGCTCCTGACCAACCTCCAGCATGAGCACA | |
| | TCGTCAAGTTCTATGGCGTCTGCGTGGAGGGCGACC | |
| | CCCTCATCATGGTCTTTGAGTACATGAAGCATGGGG | |
| | ACCTCAACAAGTTCCTCAGGGCACACGGCCCTGATG | |
| | CCGTGCTGATGGCTGAGGGCAACCCGCCCACGGAA | |
| | CTGACGCAGTCGCAGATGCTGCATATAGCCCAGCA | |
| | GATCGCCGCGGGCATGGTCTACCTGGCGTCCCAGCA | |
| | CTTCGTGCACCGCGATTTGGCCACCAGGAACTGCCT | |
| | GGTCGGGGAGAACTTGCTGGTGAAAATCGGGGACT | |
| | TTGGGATGTCCCGGGACGTGTACAGCACTGACTACT | |
| | ACAGGGTCGGTGGCCACACAATGCTGCCCATTCGCT | |
| | GGATGCCTCCAGAGAGCATCATGTACAGGAAATTC | |
| | ACGACGGAAAGCGACGTCTGGAGCCTGGGGGTCGT | |
| | GTTGTGGGAGATTTTCACCTATGGCAAACAGCCCTG | |
| | GTACCAGCTGTCAAACAATGAGGTGATAGAGTGTA | |
| | TCACTCAGGGCCGAGTCCTGCAGCGACCCCGCACGT | |
| | GCCCCCAGGAGGTGTATGAGCTGATGCTGGGGTGC | |
| | TGGCAGCGAGAGCCCCACATGAGGAAGAACATCAA | |
| | GGGCATCCATACCCTCCTTCAGAACTTGGCCAAGGC | |
| | ATCTCCGGTCTACCTGGACATTCTAGGCTAGGGCCC | |
| | TTTTCCCCAGACCGATCCTTCCCAACGTACTCCTCA | |
| | GACGGGCTGAGAGGATGAACATCTTTTAACTGCCG | |
| | CTGGAGGCCACCA | |
| SEQ ID NO: 13 | GCTCAAGCCAGACACATTTGTTCAGCACATCAAGCG | NTRK2 region |
| | ACATAACATTGTTCTGAAAAGGGAGCTAGGCGAAG | (mRNA) targeted |
| | GAGCCTTTGGAAAAGTGTTCCTAGCTGAATGCTATA | by ACD Probe No. |
| | ACCTCTGTCCTGAGCAGGACAAGATCTTGGTGGCAG | 852818 |
| | TGAAGACCCTGAAGGATGCCAGTGACAATGCACGC | |
| | AAGGACTTCCACCGTGAGGCCGAGCTCCTGACCAA | |
| | CCTCCAGCATGAGCACATCGTCAAGTTCTATGGCGT | |
| | CTGCGTGGAGGGCGACCCCCTCATCATGGTCTTTGA | |
| | GTACATGAAGCATGGGGACCTCAACAAGTTCCTCA | |
| | GGGCACACGGCCCTGATGCCGTGCTGATGGCTGAG | |
| | GGCAACCCGCCCACGGAACTGACGCAGTCGCAGAT | |
| | GCTGCATATAGCCCAGCAGATCGCCGCGGGCATGG | |
| | TCTACCTGGCGTCCCAGCACTTCGTGCACCGCGATT | |
| | TGGCCACCAGGAACTGCCTGGTCGGGGAGAACTTG | |
| | CTGGTGAAAATCGGGGACTTTGGGATGTCCCGGGA | |
| | CGTGTACAGCACTGACTACTACAGGGTCGGTGGCC | |
| | ACACAATGCTGCCCATTCGCTGGATGCCTCCAGAGA | |
| | GCATCATGTACAGGAAATTCACGACGGAAAGCGAC | |
| | GTCTGGAGCCTGGGGGTCGTGTTGTGGGAGATTTTC | |
| | ACCTATGGCAAACAGCCCTGGTACCAGCTGTCAAA | |
| | CAATGAGGTGATAGAGTGTATCACTCAGGGCCGAG | |
| | TCCTGCAGCGACCCCGCACGTGCCCCCAGGAGGTGT | |
| | ATGAGCTGATGCTGGGGTGCTGGCAGCGAGAGCCC | |
| | CACATGAGGAAGAACATCAAGGGCATCCATACCCT | |
| | CCTTCAGAACTTGGCCAAGGCATCTCCGGTCTACCT | |
| | GGACA | |
| SEQ ID NO: 14 | TCAACAAGTTCCTCAGGGCACACGGCCCTGATGCCG | NTRK2 region |
| | TGCTGATGGCTGAGGGCAACCCGCCCACGGAACTG | (mRNA) targeted |
| | ACGCAGTCGCAGATGCTGCATATAGCCCAGCAGAT | by ACD Probe No. |
| | CGCCGCGGGCATGGTCTACCTGGCGTCCCAGCACTT | 852828 |
| | CGTGCACCGCGATTTGGCCACCAGGAACTGCCTGGT | |
| | CGGGGAGAACTTGCTGGTGAAAATCGGGGACTTTG | |
| | GGATGTCCCGGGACGTGTACAGCACTGACTACTAC | |
| | AGGGTCGGTGGCCACACAATGCTGCCCATTCGCTGG | |
| | ATGCCTCCAGAGAGCATCATGTACAGGAAATTCAC | |
| | GACGGAAAGCGACGTCTGGAGCCTGGGGGTCGTGT | |
| | TGTGGGAGATTTTCACCTATGGCAAACAGCCCTGGT | |
| | ACCAGCTGTCAAACAATGAGGTGATAGAGTGTATC | |
| | ACTCAGGGCCGAGTCCTGCAGCGACCCCGCACGTG | |
| | CCCCCAGGAGGTGTATGAGCTGATGCTGGGGTGCT | |
| | GGCAGCGAGAGCCCCACATGAGGAAGAACATCAAG | |
| | GGCATCCATACCCTCCTTCAGAACTTGGCCAAGGCA | |
| | TCTCCGGTCTACCTGGACATTCTAGGCTAGGGCCCT | |
| | TTTCCCCAGACCGATCCTTCCCAACGTACTCCTCAG | |
| | ACGGGCTGAGAGGATGAACATCTTTTAACTGCCGCT | |
| | GGAGGCCACCAAGCTGCTCTCCTTCACTCTGACAGT | |
| | ATTAACATCAAAGACTCCGAGAAGCTCTCGAGGGA | |
| | AGCAGTGTGTACTTCTTCATCCATAGACACAGTATT | |
| | GACTTCTTTTTGGCATTATCTCTTTCTCTCTTTCCATC | |
| | TCCCTTGGTTGTTCCTTTTTCTTTTTTTAAATTTTCTT | |
| | TTTCTTTTTTTTTTCGTCTTCCCTGCTTCACGATTCTT | |
| | ACCCTTTCTTTTGAATCAATCTGGCTTCTGCATTACT | |
| | ATTAACTCTGCATAGACAAAGGCCTTAACAACGTA | |
| | ATTTGTTATATCAGCAGACACTCCAGTTTGCCCACC | |
| | ACAACTAACAATGCCTTGTTGTATTCCTGCCTTTGA | |
| | TGTGGATGAAAAAAAGGGAAAACAAATATTTCACT | |
| | TAAACTTTGTCACTTCTGCTGTACAGATATCGAGAG | |
| | TTTCTATGGATTCACTTCTATTTATTTATTATTATTA | |
| | CTGTTCTTATTGTTTTTGGATGGCTTAAGCCTGTGTA | |
| | TAAAAAAGAAAACTTGTGTTCAATCTGTGAAGCCTT | |
| | TATCTATGGGAGATTAAAACCAGAGAGAAAGAAGA | |
| | TTTATTATGAACCGCAATATGGGAGGAACAAAGAC | |
| | AACCACTGGGATCAGCTGGTGTCAGTCCCTACTTAG | |
| | GAAATACTCAGCAACTGTTAGCTGGGAAGAATGTA | |
| | TTCGGCACCTTCCCCTGAGGACCTTTCTGAGGAGTA | |
| | AAAAGACTACTGGCCTCTGTGCCATGGATGATTCTT | |
| | TTCCCATCACCAGAAATGATAGCGTGCAGTAGAGA | |
| | GCAAAGATGGCTTCCGTGAGACACAAGATGGCGCA | |
| | TAGTG | |
| SEQ ID NO: 19 | GAGTCCAAGATCATCCATGTGGAATACTACCAAGA | NTRK3 region |
| | GGGAGAGATTTCCGAGGGCTGCCTGCTCTTCAACAA | (mRNA) targeted |
| | GCCCACCCACTACAACAATGGCAACTATACCCTCAT | ACD Probe No. |
| | TGCCAAAAACCCACTGGGCACAGCCAACCAGACCA | 852838 mRNA |
| | TCAATGGCCACTTCCTCAAGGAGCCCTTTCCAGAGA | |
| | GCACGGATAACTTTATCTTGTTTGACGAAGTGAGTC | |
| | CCACACCTCCTATCACTGTGACCCACAAACCAGAAG | |
| | AAGACACTTTTGGGGTATCCATAGCAGTTGGACTTG | |
| | CTGCTTTTGCCTGTGTCCTGTTGGTGGTTCTCTTCGT | |
| | CATGATCAACAAATATGGTCGACGGTCCAAATTTGG | |
| | AATGAAGGGTCCCGTGGCTGTCATCAGTGGTGAGG | |
| | AGGACTCAGCCAGCCCACTGCACCACATCAACCAC | |
| | GGCATCACCACGCCCTCGTCACTGGATGCCGGGCCC | |
| | GACACTGTGGTCATTGGCATGACTCGCATCCCTGTC | |
| | ATTGAGAACCCCCAGTACTTCCGTCAGGGACACAA | |
| | CTGCCACAAGCCGGACACGTATGTGCAGCACATTA | |
| | AGAGGAGAGACATCGTGCTGAAGCGAGAACTGGGT | |
| | GAGGGAGCCTTTGGAAAGGTCTTCCTGGCCGAGTG | |
| | CTACAACCTCAGCCCGACCAAGGACAAGATGCTTG | |
| | TGGCTGTGAAGGCCCTGAAGGATCCCACCCTGGCTG | |
| | CCCGGAAGGATTTCCAGAGGGAGGCCGAGCTGCTC | |
| | ACCAACCTGCAGCATGAGCACATTGTCAAGTTCTAT | |
| | GGAGTGTGCGGCGATGGGGACCCCCTCATCATGGT | |
| | CTTTGAATACATGAAGCATGGAGACCTGAATAAGTT | |
| | CCTCAGGGCCCATGGGCCAGATGCAATGATCCTTGT | |
| | GGATGGACAGCCACGCCAGGCCAAGGGTGAGCTGG | |
| | GGCTCTCCCAAATGCTCCACATTGCCAGTCAGATCG | |
| | CCTCGGGTATGGTGTACCTGGCCTCCCAGCACTTTG | |
| | TGCACCGAGACCTGGCCACCAGGAACTGCCTGGTT | |
| | GGAGCGAATCTGCTAGTGAAGATTGGGGACTTCGG | |
| | CATGTCCAGAGATGTCTACAGCACGGATTATTACAG | |
| | GCTCTTTAATCCATCTGGAAATGATTTTTGTATATG | |
| | GTGTGAGGTGGGAGGACACACCATGCTCCCCATTC | |
| | GCTGGATGCCTCCTGAAAGCATCATGTACCGGAAGT | |
| | TCACTACAGAGAGTGATGTATGGAGCTTCGGGGTG | |
| | ATCCTCTGGGAGATCTTCACCTATGGAAAGCAGCCA | |
| | TGGTTCCAACTCTCAAACACGGAGGTCATTGAGTGC | |
| | ATTACCCAAGGTCGTGTTTTGGAGCGGCCCCGAGTC | |
| | TGCCCCAAAGAGGTGTACGATGTCATGCTGGGGTG | |
| | CTGGCAGAGGGAACCACAGCAGCGGTTGAACATCA | |
| | AGGAGATCTACAAAATCCTCCATG | |
| SEQ ID NO: 20 | GAGTCCAAGATCATCCATGTGGAATACTACCAAGA | NTRK3 region |
| | GGGAGAGATTTCCGAGGGCTGCCTGCTCTTCAACAA | (mRNA) targeted |
| | GCCCACCCACTACAACAATGGCAACTATACCCTCAT | by ACD Probe No. |
| | TGCCAAAAACCCACTGGGCACAGCCAACCAGACCA | 852848 |
| | TCAATGGCCACTTCCTCAAGGAGCCCTTTCCAGAGA | |
| | GCACGGATAACTTTATCTTGTTTGACGAAGTGAGTC | |
| | CCACACCTCCTATCACTGTGACCCACAAACCAGAAG | |
| | AAGACACTTTTGGGGTATCCATAGCAGTTGGACTTG | |
| | CTGCTTTTGCCTGTGTCCTGTTGGTGGTTCTCTTCGT | |
| | CATGATCAACAAATATGGTCGACGGTCCAAATTTGG | |
| | AATGAAGGGTCCCGTGGCTGTCATCAGTGGTGAGG | |
| | AGGACTCAGCCAGCCCACTGCACCACATCAACCAC | |
| | GGCATCACCACGCCCTCGTCACTGGATGCCGGGCCC | |
| | GACACTGTGGTCATTGGCATGACTCGCATCCCTGTC | |
| | ATTGAGAACCCCCAGTACTTCCGTCAGGGACACAA | |
| | CTGCCACAAGCCGGACACGTATGTGCAGCACATTA | |
| | AGAGGAGAGACATCGTGCTGAAGCGAGAACTGGGT | |
| | GAGGGAGCCTTTGGAAAGGTCTTCCTGGCCGAGTG | |
| | CTACAACCTCAGCCCGACCAAGGACAAGATGCTTG | |
| | TGGCTGTGAAGGCCCTGAAGGATCCCACCCTGGCTG | |
| | CCCGGAAGGATTTCCAGAGGGAGGCCGAGCTGCTC | |
| | ACCAACCTGCAGCATGAGCACATTGTCAAGTTCTAT | |
| | GGAGTGTGCGGCGATGGGGACCCCCTCATCATGGT | |
| | CTTTGAATACATGAAGCATGGAGACCTGAATAAGTT | |
| | CCTCAGGGCCCATGGGCCAGATGCAATGATCCTTGT | |
| | GGATGGACAGCCACGCCAGGCCAAGGGTGAGCTGG | |
| | GGCTCTCCCAAATGCTCCACATTGCCAGTCAGATCG | |
| | CCTCGGGTATGGTGTACCTGGCCTCCCAGCACTTTG | |
| | TGCACCGAGACCTGGCCACCAGGAACTGCCTGGTT | |
| | GGAGCGAATCTGCTAGTGAAGATTGGGGACTTCGG | |
| | CATGTCCAGAGATGTCTACAGCACGGATTATTACAG | |
| | GCTCTTTAATCCATCTGGAAATGATTTTTGTATATG | |
| | GTGTGAGGTGGGAGGACACACCATGCTCCCCATTC | |
| | GCTGGATGCCTCCTGAAAGCATCATGTACCGGAAGT | |
| | TCACTACAGAGAGTGATGTATGGAGCTTCGGGGTG | |
| | ATCCTCTGGGAGATCTTCACCTATGGAAAGCAGCCA | |
| | TGGTTCCAACTCTCAAACACGGAGGTCATTGAGTGC | |
| | ATTACCCAAGGTCGTGTTTTGGAGCGGCCCCGAGTC | |
| | TGCCCCAAAGAGGTGTACGATGTCATGCTGGGGTG | |
| | CTGGCAGAGGGAACCACAGCAGCGGTTGAACATCA | |
| | AGGAGATCTACAAAATCCTCCATGCTTTGGGGAAG | |
| | GCCACCCCAATCTACCTGGACATTCTTGGCTAGTGG | |
| | TGGCTGGTGGTCATGAATTC | |
| SEQ ID NO: 21 | CACATTAAGAGGAGAGACATCGTGCTGAAGCGAGA | NTRK3 region |
| | ACTGGGTGAGGGAGCCTTTGGAAAGGTCTTCCTGGC | (mRNA) targeted |
| | CGAGTGCTACAACCTCAGCCCGACCAAGGACAAGA | by ACD Probe No. |
| | TGCTTGTGGCTGTGAAGGCCCTGAAGGATCCCACCC | 852858 |
| | TGGCTGCCCGGAAGGATTTCCAGAGGGAGGCCGAG | |
| | CTGCTCACCAACCTGCAGCATGAGCACATTGTCAAG | |
| | TTCTATGGAGTGTGCGGCGATGGGGACCCCCTCATC | |
| | ATGGTCTTTGAATACATGAAGCATGGAGACCTGAAT | |
| | AAGTTCCTCAGGGCCCATGGGCCAGATGCAATGAT | |
| | CCTTGTGGATGGACAGCCACGCCAGGCCAAGGGTG | |
| | AGCTGGGGCTCTCCCAAATGCTCCACATTGCCAGTC | |
| | AGATCGCCTCGGGTATGGTGTACCTGGCCTCCCAGC | |
| | ACTTTGTGCACCGAGACCTGGCCACCAGGAACTGCC | |
| | TGGTTGGAGCGAATCTGCTAGTGAAGATTGGGGAC | |
| | TTCGGCATGTCCAGAGATGTCTACAGCACGGATTAT | |
| | TACAGGCTCTTTAATCCATCTGGAAATGATTTTTGT | |
| | ATATGGTGTGAGGTGGGAGGACACACCATGCTCCC | |
| | CATTCGCTGGATGCCTCCTGAAAGCATCATGTACCG | |
| | GAAGTTCACTACAGAGAGTGATGTATGGAGCTTCG | |
| | GGGTGATCCTCTGGGAGATCTTCACCTATGGAAAGC | |
| | AGCCATGGTTCCAACTCTCAAACACGGAGGTCATTG | |
| | AGTGCATTACCCAAGGTCGTGTTTTGGAGCGGCCCC | |
| | GAGTCTGCCCCAAAGAGGTGTACGATGTCATGCTG | |
| | GGGTGCTGGCAGAGGGAACCACAGCAGCGGTTGAA | |
| | CATCAAGGAGATCTACAAAATCCTCCATGCTTTGGG | |
| | GAAGGCCACCCCAATCTACCTGGACATTCTTGGCTA | |
| | GTGGTGGCTGGTGGTCATGAATTC | |

As used herein, a “target probe” is a polynucleotide that is capable of hybridizing to a target nucleic acid and capturing or binding a label probe or SGC component to that target nucleic acid. The target probe can hybridize directly to the label probe, or it can hybridize to one or more nucleic acids that in turn hybridize to the label probe; for example, the target probe can hybridize to an amplifier, a pre-amplifier or a pre-pre-amplifier in an SGC. The target probe thus includes a first polynucleotide sequence that is complementary to a polynucleotide sequence of the target nucleic acid and a second polynucleotide sequence that is complementary to a polynucleotide sequence of the label probe, amplifier, pre-amplifier, pre-pre-amplifier, or the like. The target probe is generally single stranded so that the complementary sequence is available to hybridize with a corresponding target nucleic acid, label probe, amplifier, pre-amplifier or pre-pre-amplifier. In some embodiments, the target probes are provided as a pair.

As used herein, the term “label probe” refers to an entity that binds to a target molecule, directly or indirectly, generally indirectly, and allows the target to be detected. A label probe (or “LP”) contains a nucleic acid binding portion that is typically a single stranded polynucleotide or oligonucleotide that comprises one or more labels which directly or indirectly provides a detectable signal. The label can be covalently attached to the polynucleotide, or the polynucleotide can be configured to bind to the label. For example, a biotinylated polynucleotide can bind a streptavidin-associated label. The label probe can, for example, hybridize directly to a target nucleic acid. In general, the label probe can hybridize to a nucleic acid that is in turn hybridized to the target nucleic acid or to one or more other nucleic acids that are hybridized to the target nucleic acid. Thus, the label probe can comprise a polynucleotide sequence that is complementary to a polynucleotide sequence, particularly a portion, of the target nucleic acid. Alternatively, the label probe can comprise at least one polynucleotide sequence that is complementary to a polynucleotide sequence in an amplifier, pre-amplifier, or pre-pre-amplifier in a SGC.

In some embodiments, the SGC provided herein comprises additional comments such an amplifier, a pre-amplifier, and/or a pre-pre-amplifier.

As used herein, an “amplifier” is a molecule, typically a polynucleotide, that is capable of hybridizing to multiple label probes. Typically, the amplifier hybridizes to multiple identical label probes. The amplifier can also hybridize to a target nucleic acid, to at least one target probe of a pair of target probes, to both target probes of a pair of target probes, or to nucleic acid bound to a target probe such as an amplifier, pre-amplifier or pre-pre-amplifier. For example, the amplifier can hybridize to at least one target probe and to a plurality of label probes, or to a pre-amplifier and a plurality of label probes. The amplifier can be, for example, a linear, forked, comb-like, or branched nucleic acid. As described herein for all polynucleotides, the amplifier can include modified nucleotides and/or nonstandard internucleotide linkages as well as standard deoxyribonucleotides, ribonucleotides, and/or phosphodiester bonds. Suitable amplifiers are described, for example, in U.S. Pat. Nos. 5,635,352, 5,124,246, 5,710,264, 5,849,481, and 7,709,198 and U.S. publications 2008/0038725 and 2009/0081688, each of which is incorporated by reference.

As used herein, a “pre-amplifier” is a molecule, typically a polynucleotide, that serves as an intermediate binding component between one or more target probes and one or more amplifiers. Typically, the pre-amplifier hybridizes simultaneously to one or more target probes and to a plurality of amplifiers. Exemplary pre-amplifiers are described, for example, in U.S. Pat. Nos. 5,635,352, 5,681,697 and 7,709,198 and U.S. publications 2008/0038725, 2009/0081688 and 2017/0101672, each of which is incorporated by reference.

As used herein, a “pre-pre-amplifier” is a molecule, typically a polynucleotide, that serves as an intermediate binding component between one or more target probes and one or more pre-amplifiers. Typically, the pre-pre-amplifier hybridizes simultaneously to one or more target probes and to a plurality of pre-amplifiers. Exemplary pre-pre-amplifiers are described, for example, in 2017/0101672, which is incorporated by reference.

A label is typically used in RNA in situ hybridization for detecting target nucleic acid. As used herein, a “label” is a moiety that facilitates detection of a molecule. Common labels include fluorescent, luminescent, light-scattering, and/or colorimetric labels. Suitable labels include enzymes, and fluorescent and chromogenic moieties, as well as radionuclides, substrates, cofactors, inhibitors, chemiluminescent moieties, magnetic particles, rare earth metals, metal isotopes, and the like. In a particular embodiment, the label is an enzyme. Exemplary enzyme labels include, but are not limited to Horse Radish Peroxidase (HRP), Alkaline Phosphatase (AP), β-galactosidase, glucose oxidase, and the like, as well as various proteases. Other labels include, but are not limited to, fluorophores, Dinitrophenyl (DNP), and the like. Labels are well known to those skilled in the art, as described, for example, in Hermanson, Bioconjugate Techniques, Academic Press, San Diego (1996), and U.S. Pat. Nos. 3,817,837; 3,850,752; 3,939,350; 3,996,345; 4,277,437; 4,275,149; and 4,366,241. Many labels are commercially available and can be used in methods and assays of the disclosure, including detectable enzyme/substrate combinations (Pierce, Rockford Ill.; Santa Cruz Biotechnology, Dallas Tex.; Life Technologies, Carlsbad Calif.). In a particular embodiment of the disclosure, the enzyme can utilize a chromogenic or fluorogenic substrate to produce a detectable signal, as described herein. Exemplary labels are described herein.

Any of a number of enzymes or non-enzyme labels can be utilized so long as the enzymatic activity or non-enzyme label, respectively, can be detected. The enzyme thereby produces a detectable signal, which can be utilized to detect a target nucleic acid. Particularly useful detectable signals are chromogenic or fluorogenic signals. Accordingly, particularly useful enzymes for use as a label include those for which a chromogenic or fluorogenic substrate is available. Such chromogenic or fluorogenic substrates can be converted by enzymatic reaction to a readily detectable chromogenic or fluorescent product, which can be readily detected and/or quantified using microscopy or spectroscopy. Such enzymes are well known to those skilled in the art, including but not limited to, horseradish peroxidase, alkaline phosphatase, β-galactosidase, glucose oxidase, and the like (see Hermanson, Bioconjugate Techniques, Academic Press, San Diego (1996)). Other enzymes that have well known chromogenic or fluorogenic substrates include various peptidases, where chromogenic or fluorogenic peptide substrates can be utilized to detect proteolytic cleavage reactions. The use of chromogenic and fluorogenic substrates is also well known in bacterial diagnostics, including but not limited to the use of α- and β-galactosidase, β-glucuronidase, 6-phospho-β-D-galactoside 6-phosphogalactohydrolase, β-glucosidase, α-glucosidase, amylase, neuraminidase, esterases, lipases, and the like (Manafi et al., Microbiol. Rev. 55:335-348 (1991)), and such enzymes with known chromogenic or fluorogenic substrates can readily be adapted for use in methods provided herein.

Various chromogenic or fluorogenic substrates to produce detectable signals are well known to those skilled in the art and are commercially available. Exemplary substrates that can be utilized to produce a detectable signal include, but are not limited to, 3,3′-diaminobenzidine (DAB), 3,3′,5,5′-tetramethylbenzidine (TMB), Chloronaphthol (4-CN)(4-chloro-1-naphthol), 2,2′-azino-bis(3-ethylbenzothiazoline-6-sulphonic acid) (ABTS), o-phenylenediamine dihydrochloride (OPD), and 3-amino-9-ethylcarbazole (AEC) for horseradish peroxidase; 5-bromo-4-chloro-3-indolyl-1-phosphate (BCIP), nitroblue tetrazolium (NBT), Fast Red (Fast Red TR/AS-MX), and p-Nitrophenyl Phosphate (PNPP) for alkaline phosphatase; 1-Methyl-3-indolyl-β-D-galactopyranoside and 2-Methoxy-4-(2-nitrovinyl)phenyl β-D-galactopyranoside for β-galactosidase; 2-Methoxy-4-(2-nitrovinyl)phenyl β-D-glucopyranoside for β-glucosidase; and the like. Exemplary fluorogenic substrates include, but are not limited to, 4-(Trifluoromethyl)umbelliferyl phosphate for alkaline phosphatase; 4-Methylumbelliferyl phosphate bis (2-amino-2-methyl-1,3-propanediol), 4-Methylumbelliferyl phosphate bis (cyclohexylammonium) and 4-Methylumbelliferyl phosphate for phosphatases; QuantaBlu™ and Quintolet for horseradish peroxidase; 4-Methylumbelliferyl β-D-galactopyranoside, Fluorescein di(β-D-galactopyranoside) and Naphthofluorescein di-(β-D-galactopyranoside) for 3-galactosidase; 3-Acetylumbelliferyl β-D-glucopyranoside and 4-Methylumbelliferyl-β-D-glucopyranoside for β-glucosidase; and 4-Methylumbelliferyl-α-D-galactopyranoside for α-galactosidase. Exemplary enzymes and substrates for producing a detectable signal are also described, for example, in US publication 2012/0100540. Various detectable enzyme substrates, including chromogenic or fluorogenic substrates, are well known and commercially available (Pierce, Rockford Ill.; Santa Cruz Biotechnology, Dallas Tex.; Invitrogen, Carlsbad Calif.; 42 Life Science; Biocare). Generally, the substrates are converted to products that form precipitates that are deposited at the site of the target nucleic acid. Other exemplary substrates include, but are not limited to, HRP-Green (42 Life Science), Betazoid DAB, Cardassian DAB, Romulin AEC, Bajoran Purple, Vina Green, Deep Space Black™, Warp Red™, Vulcan Fast Red and Ferangi Blue from Biocare (Concord Calif.; biocare.net/products/detection/chromogens).

Exemplary rare earth metals and metal isotopes suitable as a detectable label include, but are not limited to, lanthanide (III) isotopes such as 141Pr, 142Nd, 143Nd, 144Nd, 145Nd, 146Nd, 147Sm, 148Nd, 149Sm, 150Nd, 151Eu, 152Sm, 153Eu, 154Sm, 155Gd, 156Gd, 158Gd, 159Tb, 160Gd, 161Dy, 162Dy, 163Dy, 164Dy, 165Ho, 166Er, 167Er, 168Er, 169Tm, 170Er, 171Yb, 172Yb, 173Yb, 174Yb, 175Lu, and 176Yb. Metal isotopes can be detected, for example, using time-of-flight mass spectrometry (TOF-MS) (for example, Fluidigm Helios and Hyperion systems, fluidigm.com/systems; South San Francisco, Calif.).

Biotin-avidin (or biotin-streptavidin) is a well-known signal amplification system based on the fact that the two molecules have extraordinarily high affinity to each other and that one avidin/streptavidin molecule can bind four biotin molecules. Antibodies are widely used for signal amplification in immunohistochemistry and ISH. Tyramide signal amplification (TSA) is based on the deposition of a large number of haptenized tyramide molecules by peroxidase activity. Tyramine is a phenolic compound. In the presence of small amounts of hydrogen peroxide, immobilized Horse Radish Peroxidase (HRP) converts the labeled substrate into a short-lived, extremely reactive intermediate. The activated substrate molecules then very rapidly react with and covalently bind to electron-rich moieties of proteins, such as tyrosine, at or near the site of the peroxidase binding site. In this way, many hapten molecules conjugated to tyramide can be introduced at the hybridization site in situ. Subsequently, the deposited tyramide-hapten molecules can be visualized directly or indirectly. Such a detection system is described in more detail, for example, in U.S. publication 2012/0100540.

Embodiments described herein can utilize enzymes to generate a detectable signal using appropriate chromogenic or fluorogenic substrates. It is understood that, alternatively, a label probe can have a detectable label directly coupled to the nucleic acid portion of the label probe. Exemplary detectable labels are well known to those skilled in the art, including but not limited to chromogenic or fluorescent labels (see Hermanson, Bioconjugate Techniques, Academic Press, San Diego (1996)). Exemplary fluorophores useful as labels include, but are not limited to, rhodamine derivatives, for example, tetramethylrhodamine, rhodamine B, rhodamine 6G, sulforhodamine B, Texas Red (sulforhodamine 101), rhodamine 110, and derivatives thereof such as tetramethylrhodamine-5-(or 6), lissamine rhodamine B, and the like; 7-nitrobenz-2-oxa-1,3-diazole (NBD); fluorescein and derivatives thereof; napthalenes such as dansyl (5-dimethylaminonapthalene-1-sulfonyl); coumarin derivatives such as 7-amino-4-methylcoumarin-3-acetic acid (AMCA), 7-diethylamino-3-[(4′-(iodoacetyl)amino)phenyl]-4-methylcoumarin (DCIA), Alexa fluor dyes (Molecular Probes), and the like; 4,4-difluoro-4-bora-3a,4a-diaza-s-indacene (BODIPY™) and derivatives thereof (Molecular Probes; Eugene, Oreg.); pyrenes and sulfonated pyrenes such as Cascade Blue′ and derivatives thereof, including 8-methoxypyrene-1,3,6-trisulfonic acid, and the like; pyridyloxazole derivatives and dapoxyl derivatives (Molecular Probes); Lucifer Yellow (3,6-disulfonate-4-amino-naphthalimide) and derivatives thereof; CyDye™ fluorescent dyes (Amersham/GE Healthcare Life Sciences; Piscataway N.J.), ATTO 390, DyLight 395XL, ATTO 425, ATTO 465, ATTO 488, ATTO 490LS, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 643, ATTO 647, ATTO 647N, ATTO 655, ATTO Oxa12, ATTO 665, ATTO 680, ATTO 700, ATTO 725, ATTO 740, Cyan 500 NETS-Ester (ATTO-TECH, Siegen, Germany), and the like. Exemplary chromophores include, but are not limited to, phenolphthalein, malachite green, nitroaromatics such as nitrophenyl, diazo dyes, dabsyl (4-dimethylaminoazobenzene-4′-sulfonyl), and the like.

As disclosed herein, the methods provided herein can utilize concurrent detection of multiple target nucleic acids. In the case of using fluorophores as labels, the fluorophores to be used for detection of multiple target nucleic acids are selected so that each of the fluorophores are distinguishable and can be detected concurrently in the fluorescence microscope in the case of concurrent detection of target nucleic acids. Such fluorophores are selected to have spectral separation of the emissions so that distinct labeling of the target nucleic acids can be detected concurrently. Methods of selecting suitable distinguishable fluorophores for use in methods of the disclosure are well known in the art (see, for example, Johnson and Spence, “Molecular Probes Handbook, a Guide to Fluorescent Probes and Labeling Technologies, 11th ed., Life Technologies (2010)).

Well known methods such as microscopy, cytometry (e.g., mass cytometry, cytometry by time of flight (CyTOF), flow cytometry), or spectroscopy can be utilized to visualize chromogenic, fluorescent, or metal detectable signals associated with the respective target nucleic acids. In general, either chromogenic substrates or fluorogenic substrates, or chromogenic or fluorescent labels, or rare earth metal isotopes, will be utilized for a particular assay, if different labels are used in the same assay, so that a single type of instrument can be used for detection of nucleic acid targets in the same sample.

As disclosed herein, the label can be designed such that the labels are optionally cleavable. As used herein, a cleavable label refers to a label that is attached or conjugated to a label probe so that the label can be removed, for example, in order to use the same label in a subsequent round of labeling and detecting of target nucleic acids. Generally, the labels are conjugated to the label probe by a chemical linker that is cleavable. Methods of conjugating a label to a label probe so that the label is cleavable are well known to those skilled in the art (see, for example, Hermanson, Bioconjugate Techniques, Academic Press, San Diego (1996); Daniel et al., BioTechniques 24(3):484-489 (1998)). One particular system of labeling oligonucleotides is the FastTag system (Daniel et al., supra, 1998; Vector Laboratories, Burlinghame Calif.). Various cleavable moieties can be included in the linker so that the label can be cleaved from the label probe. Such cleavable moieties include groups that can be chemically, photo chemically or enzymatically cleaved. Cleavable chemical linkers can include a cleavable chemical moiety, such as disulfides, which can be cleaved by reduction, glycols or diols, which can be cleaved by periodate, diazo bonds, which can be cleaved by dithionite, esters, which can be cleaved by hydroxylamine, sulfones, which can be cleaved by base, and the like (see Hermanson, supra, 1996). One particularly useful cleavable linker is a linker containing a disulfide bond, which can be cleaved by reducing the disulfide bond. In other embodiments, the linker can include a site for cleavage by an enzyme. For example, the linker can contain a proteolytic cleavage site. Generally, such a cleavage site is for a sequence-specific protease. Such proteases include, but are not limited to, human rhinovirus 3C protease (cleavage site LEVLFQ/GP), enterokinase (cleavage site DDDDK/), factor Xa (cleavage site IEGR/), tobacco etch virus protease (cleavage site ENLYFQ/G), and thrombin (cleavage site LVPR/GS) (see, for example, Oxford Genetics, Oxford, UK). Another cleavable moiety can be, for example, uracil-DNA (DNA containing uracil), which can be cleaved by uracil-DNA glycosylase (UNG) (see, for example, Sidorenko et al., FEBS Lett. 582(3):410-404 (2008)).

The cleavable labels can be removed by applying an agent, such as a chemical agent or light, to cleave the label and release it from the label probe. As discussed above, useful cleaving agents for chemical cleavage include, but are not limited to, reducing agents, periodate, dithionite, hydroxylamine, base, and the like (see Hermanson, supra, 1996). One useful method for cleaving a linker containing a disulfide bond is the use of tris(2-carboxyethyl)phosphine (TCEP) (see Moffitt et al., Proc. Natl. Acad. Sci. USA 113:11046-11051 (2016)). In one embodiment, TCEP is used as an agent to cleave a label from a label probe.

For methods of the present disclosure for RNA in situ detection of nucleic acid targets in a cell, the cell is optionally fixed and/or permeabilized before hybridization of the target probes. Fixing and permeabilizing cells can facilitate retaining the nucleic acid targets in the cell and permit the target probes, label probes, and so forth, to enter the cell and reach the target nucleic acid molecule. The cell is optionally washed to remove materials not captured to a nucleic acid target. The cell can be washed after any of various steps, for example, after hybridization of the target probes to the nucleic acid targets to remove unbound target probes, and the like. Methods for fixing and permeabilizing cells for in situ detection of nucleic acids, as well as methods for hybridizing, washing and detecting target nucleic acids, are also well known in the art (see, for example, US 2008/0038725; US 2009/0081688; Hicks et al., J. Mol. Histol. 35:595-601 (2004); Stoler, Clinics in Laboratory Medicine 10(1):215-236 (1990); In situ hybridization. A practical approach, Wilkinson, ed., IRL Press, Oxford (1992); Schwarzacher and Heslop-Harrison, Practical in situ hybridization, BIOS Scientific Publishers Ltd, Oxford (2000); Shapiro, Practical Flow Cytometry 3rd ed., Wiley-Liss, New York (1995); Ormerod, Flow Cytometry, 2nd ed., Springer (1999)). Exemplary fixing agents include, but are not limited to, aldehydes (formaldehyde, glutaraldehyde, and the like), acetone, alcohols (methanol, ethanol, and the like). Exemplary permeabilizing agents include, but are not limited to, alcohols (methanol, ethanol, and the like), acids (glacial acetic acid, and the like), detergents (Triton, NP-40, Tween™ 20, and the like), saponin, digitonin, Leucoperm™ (BioRad, Hercules, Calif.), and enzymes (for example, lysozyme, lipases, proteases and peptidases). Permeabilization can also occur by mechanical disruption, such as in tissue slices.

RNA in situ detection methods can be used on tissue specimens immobilized on a glass slide, on single cells in suspension such as peripheral blood mononucleated cells (PBMCs) isolated from blood samples, and the like. Tissue specimens include, for example, tissue biopsy samples. Blood samples include, for example, blood samples taken for diagnostic purposes. In the case of a blood sample, the blood can be directly analyzed, such as in a blood smear, or the blood can be processed, for example, lysis of red blood cells, isolation of PBMCs or leukocytes, isolation of target cells, and the like, such that the cells in the sample analyzed by methods of the disclosure are in a blood sample or are derived from a blood sample. Similarly, a tissue specimen can be processed, for example, the tissue specimen minced and treated physically or enzymatically to disrupt the tissue into individual cells or cell clusters. Additionally, a cytological sample can be processed to isolate cells or disrupt cell clusters, if desired. Thus, the tissue, blood and cytological samples can be obtained and processed using methods well known in the art. The methods of the disclosure can be used in diagnostic applications to identify the presence or absence of pathological cells based on the presence or absence of a nucleic acid target that is a biomarker indicative of a pathology.

It is understood by those skilled in the art that any of a number of suitable samples can be used for detecting target nucleic acids using methods provided herein. The sample for use in methods provided herein will generally be a biological sample or tissue sample. Such a sample can be obtained from a biological subject, including a sample of biological tissue or fluid origin that is collected from an individual or some other source of biological material such as biopsy, autopsy or forensic materials. A biological sample also includes samples from a region of a biological subject containing or suspected of containing precancerous or cancer cells or tissues, for example, a tissue biopsy, including fine needle aspirates, blood sample or cytological specimen. Such samples can be, but are not limited to, organs, tissues, tissue fractions and/or cells isolated from an organism such as a mammal. Exemplary biological samples include, but are not limited to, a cell culture, including a primary cell culture, a cell line, a tissue, an organ, an organelle, a biological fluid, and the like. Additional biological samples include but are not limited to a skin sample, tissue biopsies, including fine needle aspirates, cytological samples, stool, bodily fluids, including blood and/or serum samples, saliva, semen, and the like. Such samples can be used for medical or veterinary diagnostic purposes.

Collection of cytological samples for analysis by methods provided herein are well known in the art (see, for example, Dey, “Cytology Sample Procurement, Fixation and Processing” in Basic and Advanced Laboratory Techniques in Histopathology and Cytology pp. 121-132, Springer, Singapore (2018); “Non-Gynecological Cytology Practice Guideline” American Society of Cytopathology, Adopted by the ASC executive board Mar. 2, 2004).

For example, methods for processing samples for analysis of cervical tissue, including tissue biopsy and cytology samples, are well known in the art (see, for example, Cecil Textbook of Medicine, Bennett and Plum, eds., 20th ed., WB Saunders, Philadelphia (1996); Colposcopy and Treatment of Cervical Intraepithelial Neoplasia: A Beginner's Manual, Sellors and Sankaranarayanan, eds., International Agency for Research on Cancer, Lyon, France (2003); Kalaf and Cooper, J. Clin. Pathol. 60:449-455 (2007); Brown and Trimble, Best Pract Res. Clin. Obstet. Gynaecol. 26:233-242 (2012); Waxman et al., Obstet. Gynecol. 120:1465-1471 (2012); Cervical Cytology Practice Guidelines TOC, Approved by the American Society of Cytopathology (ASC) Executive Board, Nov. 10, 2000)).

In particular embodiments, the sample is a tissue specimen or is derived from a tissue specimen. In some embodiments, the tissue specimen is formalin-fixed paraffin-embedded (FFPE). In some embodiments, the tissue specimen is fresh frozen. In some embodiments, the tissue specimen is prepared with a fixative other than formalin. In some embodiments, the fixative other than formalin is selected from the group consisting of ethanol, methanol, Bouin's, B5, and I.B.F. In other particular embodiments, the sample is a blood sample or is derived from a blood sample. In still other particular embodiments, the sample is a cytological sample or is derived from a cytological sample.

### 7.4. Methods of Diagnosing

In another aspect, provided herein is a method of determining if a subject has cancer or is likely to develop cancer comprising detecting a NTRK fusion gene in a sample by RNA in situ hybridization using the methods as described above.

In some embodiments, the presence of one or more NTRK fusion gene as determined by the methods provided herein indicates that the subject has cancer or is likely to develop cancer.

In some embodiments, the method provided herein can be used for diagnosing cancer in a subject. In some embodiments, the cancer is selected from a group consisting of mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, the cancer is a hematological cancer, such as leukemia, lymphoma, or myeloma. In some embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CIVIL), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the cancer is a solid tumor cancer. In some embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer.

In certain embodiments, the cancer is selected from a group consisting of colorectal cancer (CRC), papillary thyroid cancer (PTC), non-small-cell lung carcinoma (NSCLC), sarcoma, pediatric glioma, breast cancer, gallbladder, cholangiocarcinoma, spitzoid melanoma, astrocytoma, glioblastoma (GBM), pancreatic cancer, uterus carcinoma, pilocytic astrocytoma, pediatric glioma, head and neck squamous cell carcinoma (HNSCC), glioma, salivary gland tumor (including acinic cell carcinoma), adult acute myeloid leukemia (AML), nephroma, and inflammatory myofibroblastic tumor (IMT).

The best-known form of NTRK fusion gene is the ETV6-NTRK3, which is present in >95% of secretory carcinomas of the breast. See, e.g., Tognon et al., Cancer Cell, 2(5): 367 (2002), and of the salivary glands (i.e. mammary analogue secretory carcinoma of the salivary glands) See, e.g., Skalova et al., the American Journal of Surgical Pathology, 34: 599 (2010), congenital fibrosarcoma See, e.g., Knezevich et al., Nature Genetics, 18(2): 184 (1998), and cellular mesoblastic nephromas. See, e.g., Knezevich et al., Cancer Research, 58: 5046 (1998). This fusion gene is the product of the t(12;15)(p13;q25) chromosomal translocation, which results in a chimeric transcript encompassing exon 4, 5 or 6 of ETV6 and the kinase domain of NTRK3. See, e.g., Tognon et al., Cancer Cell, 2(5): 367 (2002). The ETV6-NTRK3 fusion gene leads to constitutive activation of the TRKC kinase domain, with downstream activation of the PI3K/AKT and MAPK pathways.

TRK gene fusions are found across several adult and pediatric tumor types and across diverse tissue and cell lineages. However, the prevalence of the NTRK gene fusions is dramatically different among tumor types, with some are rare in common solid tumors with a frequency of <1%, but occur more frequently in some rare pediatric and adult tumor types, with a frequency of up to 100% in infantile fibrosarcoma and secretory breast cancer.

Accordingly, NTRK gene fusions can be categorized into two groups according to the frequency at which they are detected. One group consists of rare cancers which are enriched for NTRK gene fusion to the extent that these alterations can be considered diagnostic features of the tumor. These are also known as high-prevalence tumors.

In some embodiments, the method of detecting a NTRK fusion gene disclosed herein comprises obtaining a sample from a subject having or who is likely to develop a high-prevalence tumor. In one embodiment, the method of detecting a NTRK fusion gene disclosed herein comprises obtaining a sample from a subject having or who is likely to develop breast secretory carcinoma, approximately 95% of which has NTRK gene fusion, especially NTRK3 gene fusion. In one embodiment, the method of detecting a NTRK fusion gene disclosed herein comprises obtaining a sample from a subject having or who is likely to develop infantile (congenital) fibrosarcoma, approximately 95% of which has NTRK gene fusion, especially NTRK3 gene fusion. In one embodiment, the method of detecting a NTRK fusion gene disclosed herein comprises obtaining a sample from a subject having or who is likely to develop mammary analogue secretory carcinoma of salivary glands, approximately 90% of which has NTRK gene fusion, especially NTRK3 gene fusion. In one embodiment, the method of detecting a NTRK fusion gene disclosed herein comprises obtaining a sample from a subject having or who is likely to develop congenital mesoblastic nephroma, approximately 70% of which has NTRK gene fusion, especially NTRK3 gene fusion. In one embodiment, the method of detecting a NTRK fusion gene disclosed herein comprises obtaining a sample from a subject having or who is likely to develop spitz tumors and spitzoid melanoma, approximately 15% of which has NTRK gene fusion, especially NTRK1 gene fusion.

In some embodiments, the method disclosed herein comprises determining if a subject has or is likely to develop a high-prevalence tumor. In one embodiment, the method disclosed herein comprises determining if a subject has or is likely to develop breast secretory carcinoma, approximately 95% of which has NTRK gene fusion, especially NTRK3 gene fusion. In one embodiment, the method disclosed herein comprises determining if a subject has or is likely to develop infantile (congenital) fibrosarcoma, approximately 95% of which has NTRK gene fusion, especially NTRK3 gene fusion. In one embodiment, the method disclosed herein comprises determining if a subject has or is likely to develop mammary analogue secretory carcinoma of salivary glands, approximately 90% of which has NTRK gene fusion, especially NTRK3 gene fusion. In one embodiment, the method disclosed herein comprises determining if a subject has or is likely to develop congenital mesoblastic nephroma, approximately 70% of which has NTRK gene fusion, especially NTRK3 gene fusion. In one embodiment, the method disclosed herein comprises determining if a subject has or is likely to develop spitz tumors and spitzoid melanoma, approximately 15% of which has NTRK gene fusion, especially NTRK1 gene fusion.

The other group of NTRK related tumors consists of more common cancers (e.g. lung cancer), in which NTRK gene fusion are rare, which are also known as low-prevalence tumors. The rarity of NTRK gene fusion in the low-prevalence tumors has made it challenging to detect TRK fusion proteins in the scattered incidence across various tumor types, especially if the detection method employed, for example, has long turnaround time, has to be done in certain facility, costly with poor reimbursement from insurance companies, and/or requires many different specifically designed probes.

In some embodiments, the method of detecting a NTRK fusion gene disclosed herein comprises obtaining a sample from a subject having or who is likely to develop a low-prevalence tumor. In one embodiment, the method of detecting a NTRK fusion gene disclosed herein comprises obtaining a sample from a subject having or who is likely to develop spitz tumors and spitzoid melanoma. In one embodiment, the method of detecting a NTRK fusion gene disclosed herein comprises obtaining a sample from a subject having or who is likely to develop papillary thyroid carcinoma. In one embodiment, the method of detecting a NTRK fusion gene disclosed herein comprises obtaining a sample from a subject having or who is likely to develop intrahepatic cholangiocarcinoma. In one embodiment, the method of detecting a NTRK fusion gene disclosed herein comprises obtaining a sample from a subject having or who is likely to develop astrocytoma. In one embodiment, the method of detecting a NTRK fusion gene disclosed herein comprises obtaining a sample from a subject having or who is likely to develop appendiceal adenocarcinoma. In one embodiment, the method of detecting a NTRK fusion gene disclosed herein comprises obtaining a sample from a subject having or who is likely to develop pediatric DIPG and non-brainstem high-grade glioma. In one embodiment, the method of detecting a NTRK fusion gene disclosed herein comprises obtaining a sample from a subject having or who is likely to develop uterine sarcoma. In one embodiment, the method of detecting a NTRK fusion gene disclosed herein comprises obtaining a sample from a subject having or who is likely to develop glioblastoma. In one embodiment, the method of detecting a NTRK fusion gene disclosed herein comprises obtaining a sample from a subject having or who is likely to develop thyroid carcinoma. In one embodiment, the method of detecting a NTRK fusion gene disclosed herein comprises obtaining a sample from a subject having or who is likely to develop sarcoma (NOS). In one embodiment, the method of detecting a NTRK fusion gene disclosed herein comprises obtaining a sample from a subject having or who is likely to develop GIST. In one embodiment, the method of detecting a NTRK fusion gene disclosed herein comprises obtaining a sample from a subject having or who is likely to develop lung adenocarcinoma. In one embodiment, the method of detecting a NTRK fusion gene disclosed herein comprises obtaining a sample from a subject having or who is likely to develop ph-like acute lymphoblastic leukemia. In one embodiment, the method of detecting a NTRK fusion gene disclosed herein comprises obtaining a sample from a subject having or who is likely to develop colon adenocarcinoma. In one embodiment, the method of detecting a NTRK fusion gene disclosed herein comprises obtaining a sample from a subject having or who is likely to develop brain low-grade glioma. In one embodiment, the method of detecting a NTRK fusion gene disclosed herein comprises obtaining a sample from a subject having or who is likely to develop melanoma (skin cutaneous). In one embodiment, the method of detecting a NTRK fusion gene disclosed herein comprises obtaining a sample from a subject having or who is likely to develop head and neck squamous cell carcinoma. In one embodiment, the method of detecting a NTRK fusion gene disclosed herein comprises obtaining a sample from a subject having or who is likely to develop breast invasive carcinoma.

In some embodiments, the method of detecting a NTRK fusion gene in a low-prevalence tumor with RNA ISH as disclosed further comprises following up with detecting a NTRK fusion gene with another detection technology. In some embodiments, the RNA ISH serves as an initial screening tool to enrich the NTRK positive samples before another detection technology for NTRK fusion gene is employed. In one embodiment, the method of detecting a NTRK fusion gene in spitz tumors and spitzoid with RNA ISH as disclosed further comprises following up with detecting a NTRK fusion gene with another detection technology, wherein RNA ISH as disclosed is a screening tool. In one embodiment, the method of detecting a NTRK fusion gene in papillary thyroid carcinoma with RNA ISH as disclosed further comprises following up with detecting a NTRK fusion gene with another detection technology, wherein RNA ISH as disclosed is a screening tool. In one embodiment, the method of detecting a NTRK fusion gene in intrahepatic cholangiocarcinoma with RNA ISH as disclosed further comprises following up with detecting a NTRK fusion gene with another detection technology, wherein RNA ISH as disclosed is a screening tool. In one embodiment, the method of detecting a NTRK fusion gene in astrocytoma with RNA ISH as disclosed further comprises following up with detecting a NTRK fusion gene with another detection technology, wherein RNA ISH as disclosed is a screening tool. In one embodiment, the method of detecting a NTRK fusion gene in appendiceal adenocarcinoma with RNA ISH as disclosed further comprises following up with detecting a NTRK fusion gene with another detection technology, wherein RNA ISH as disclosed is a screening tool. In one embodiment, the method of detecting a NTRK fusion gene in pediatric DIPG and non-brainstem high-grade glioma with RNA ISH as disclosed further comprises following up with detecting a NTRK fusion gene with another detection technology, wherein RNA ISH as disclosed is a screening tool. In one embodiment, the method of detecting a NTRK fusion gene in uterine sarcoma with RNA ISH as disclosed further comprises following up with detecting a NTRK fusion gene with another detection technology, wherein RNA ISH as disclosed is a screening tool. In one embodiment, the method of detecting a NTRK fusion gene in glioblastoma with RNA ISH as disclosed further comprises following up with detecting a NTRK fusion gene with another detection technology, wherein RNA ISH as disclosed is a screening tool. In one embodiment, the method of detecting a NTRK fusion gene in thyroid carcinoma with RNA ISH as disclosed further comprises following up with detecting a NTRK fusion gene with another detection technology, wherein RNA ISH as disclosed is a screening tool. In one embodiment, the method of detecting a NTRK fusion gene in sarcoma (NOS) with RNA ISH as disclosed further comprises following up with detecting a NTRK fusion gene with another detection technology, wherein RNA ISH as disclosed is a screening tool. In one embodiment, the method of detecting a NTRK fusion gene in GIST with RNA ISH as disclosed further comprises following up with detecting a NTRK fusion gene with another detection technology, wherein RNA ISH as disclosed is a screening tool. In one embodiment, the method of detecting a NTRK fusion gene in lung adenocarcinoma with RNA ISH as disclosed further comprises following up with detecting a NTRK fusion gene with another detection technology, wherein RNA ISH as disclosed is a screening tool. In one embodiment, the method of detecting a NTRK fusion gene in ph-like acute lymphoblastic leukemia with RNA ISH as disclosed further comprises following up with detecting a NTRK fusion gene with another detection technology, wherein RNA ISH as disclosed is a screening tool. In one embodiment, the method of detecting a NTRK fusion gene in colon adenocarcinoma with RNA ISH as disclosed further comprises following up with detecting a NTRK fusion gene with another detection technology, wherein RNA ISH as disclosed is a screening tool. In one embodiment, the method of detecting a NTRK fusion gene in brain low-grade glioma with RNA ISH as disclosed further comprises following up with detecting a NTRK fusion gene with another detection technology, wherein RNA ISH as disclosed is a screening tool. In one embodiment, the method of detecting a NTRK fusion gene in melanoma (skin cutaneous) with RNA ISH as disclosed further comprises following up with detecting a NTRK fusion gene with another detection technology, wherein RNA ISH as disclosed is a screening tool. In one embodiment, the method of detecting a NTRK fusion gene in head and neck squamous cell carcinoma with RNA ISH as disclosed further comprises following up with detecting a NTRK fusion gene with another detection technology, wherein RNA ISH as disclosed is a screening tool. In one embodiment, the method of detecting a NTRK fusion gene in breast invasive carcinoma with RNA ISH as disclosed further comprises following up with detecting a NTRK fusion gene with another detection technology, wherein RNA ISH as disclosed is a screening tool.

In some embodiments, the method disclosed herein comprises determining if a subject has or is likely to develop a low-prevalence tumor. In one embodiment, the method disclosed herein comprises determining if a subject has or is likely to develop spitz tumors and spitzoid melanoma. In one embodiment, the method disclosed herein comprises determining if a subject has or is likely to develop papillary thyroid carcinoma. In one embodiment, the method disclosed herein comprises determining if a subject has or is likely to develop intrahepatic cholangiocarcinoma. In one embodiment, the method disclosed herein comprises determining if a subject has or is likely to develop astrocytoma. In one embodiment, the method disclosed herein comprises determining if a subject has or is likely to develop appendiceal adenocarcinoma. In one embodiment, the method disclosed herein comprises determining if a subject has or is likely to develop pediatric DIPG and non-brainstem high-grade glioma. In one embodiment, the method disclosed herein comprises determining if a subject has or is likely to develop uterine sarcoma. In one embodiment, the method disclosed herein comprises determining if a subject has or is likely to develop glioblastoma. In one embodiment, the method disclosed herein comprises determining if a subject has or is likely to develop thyroid carcinoma. In one embodiment, the method disclosed herein comprises determining if a subject has or is likely to develop sarcoma (NOS). In one embodiment, the method disclosed herein comprises determining if a subject has or is likely to develop GIST. In one embodiment, the method disclosed herein comprises determining if a subject has or is likely to develop lung adenocarcinoma. In one embodiment, the method disclosed herein comprises determining if a subject has or is likely to develop ph-like acute lymphoblastic leukemia. In one embodiment, the method disclosed herein comprises determining if a subject has or is likely to develop colon adenocarcinoma. In one embodiment, the method disclosed herein comprises determining if a subject has or is likely to develop brain low-grade glioma. In one embodiment, the method disclosed herein comprises determining if a subject has or is likely to develop melanoma (skin cutaneous). In one embodiment, the method disclosed herein comprises determining if a subject has or is likely to develop head and neck squamous cell carcinoma. In one embodiment, the method disclosed herein comprises determining if a subject has or is likely to develop breast invasive carcinoma.

In some embodiments, the method disclosed herein comprises determining if a subject has or is likely to develop a low-prevalence tumor further comprises following up with another detection technology for NTRK gene fusion after detection with RNA ISH disclosed herein. In one embodiment, the method disclosed herein comprises determining if a subject has or is likely to develop spitz tumors and spitzoid melanoma further comprises following up with another detection technology for NTRK gene fusion after detection with RNA ISH disclosed herein. In one embodiment, the method disclosed herein comprises determining if a subject has or is likely to develop papillary thyroid carcinoma further comprises following up with another detection technology for NTRK gene fusion after detection with RNA ISH disclosed herein. In one embodiment, the method disclosed herein comprises determining if a subject has or is likely to develop intrahepatic cholangiocarcinoma further comprises following up with another detection technology for NTRK gene fusion after detection with RNA ISH disclosed herein. In one embodiment, the method disclosed herein comprises determining if a subject has or is likely to develop astrocytoma further comprises following up with another detection technology for NTRK gene fusion after detection with RNA ISH disclosed herein. In one embodiment, the method disclosed herein comprises determining if a subject has or is likely to develop appendiceal adenocarcinoma further comprises following up with another detection technology for NTRK gene fusion after detection with RNA ISH disclosed herein. In one embodiment, the method disclosed herein comprises determining if a subject has or is likely to develop pediatric DIPG and non-brainstem high-grade glioma further comprises following up with another detection technology for NTRK gene fusion after detection with RNA ISH disclosed herein. In one embodiment, the method disclosed herein comprises determining if a subject has or is likely to develop uterine sarcoma further comprises following up with another detection technology for NTRK gene fusion after detection with RNA ISH disclosed herein. In one embodiment, the method disclosed herein comprises determining if a subject has or is likely to develop glioblastoma further comprises following up with another detection technology for NTRK gene fusion after detection with RNA ISH disclosed herein. In one embodiment, the method disclosed herein comprises determining if a subject has or is likely to develop thyroid carcinoma further comprises following up with another detection technology for NTRK gene fusion after detection with RNA ISH disclosed herein. In one embodiment, the method disclosed herein comprises determining if a subject has or is likely to develop sarcoma (NOS) further comprises following up with another detection technology for NTRK gene fusion after detection with RNA ISH disclosed herein. In one embodiment, the method disclosed herein comprises determining if a subject has or is likely to develop GIST further comprises following up with another detection technology for NTRK gene fusion after detection with RNA ISH disclosed herein. In one embodiment, the method disclosed herein comprises determining if a subject has or is likely to develop lung adenocarcinoma further comprises following up with another detection technology for NTRK gene fusion after detection with RNA ISH disclosed herein. In one embodiment, the method disclosed herein comprises determining if a subject has or is likely to develop ph-like acute lymphoblastic leukemia further comprises following up with another detection technology for NTRK gene fusion after detection with RNA ISH disclosed herein. In one embodiment, the method disclosed herein comprises determining if a subject has or is likely to develop colon adenocarcinoma further comprises following up with another detection technology for NTRK gene fusion after detection with RNA ISH disclosed herein. In one embodiment, the method disclosed herein comprises determining if a subject has or is likely to develop brain low-grade glioma further comprises following up with another detection technology for NTRK gene fusion after detection with RNA ISH disclosed herein. In one embodiment, the method disclosed herein comprises determining if a subject has or is likely to develop melanoma (skin cutaneous) further comprises following up with another detection technology for NTRK gene fusion after detection with RNA ISH disclosed herein. In one embodiment, the method disclosed herein comprises determining if a subject has or is likely to develop head and neck squamous cell carcinoma further comprises following up with another detection technology for NTRK gene fusion after detection with RNA ISH disclosed herein. In one embodiment, the method disclosed herein comprises determining if a subject has or is likely to develop breast invasive carcinoma further comprises following up with another detection technology for NTRK gene fusion after detection with RNA ISH disclosed herein.

The fusions are more common in the NTRK1 and NTRK3 genes, and less frequent in NTRK2. See, e.g., Chen et al., Journal of Hematology & Oncology, 11(78): 1(2018); and Lassen, ESMO, 4:e000612: 1(2019). Therefore, if a detection technology is intrinsically poor at detecting one or more of the NTRK family member(s), it will cast a significant doubt on at least the sensitivity of the detection technology. For example, for immunohistochemistry (IHC), the available TRK antibodies have much weaker staining in tumors harboring NTRK3 fusions than tumors with NTRK1/NTRK2 fusions. Therefore, a sample with true NTRK3 fusions might be detected by IHC as NTRK-fusion negative, which impairs the sensitivity and negative predictive value of IHC. According to the current practice and expert recommendation of IHC (see, e.g., Marchio et al., Annals of Oncology, 30: 1417(2019).), IHC is being used as a screening tool before other more confirmative detection methods are employed. However, the above-mentioned low sensitivity and negative predictive value will significantly compromise IHC serving as a qualified screening tool. The present methods provide advantages particularly in this regard.

### 7.5. Methods of Treating

In another aspect, provided here is a selective treatment method comprising administering a NTRK kinase inhibitor to a subject determined to have a NTRK fusion gene using the method provided here (e.g., in Sections 7.3 and 7.4 above).

In some embodiments, provided herein is a method of treating cancer comprising administering a NTRK kinase inhibitor to a subject determined to have a NTRK1 fusion gene. In some embodiments, provided herein is a method of treating cancer comprising administering a NTRK kinase inhibitor to a subject determined to have a NTRK2 fusion gene. In some embodiments, provided herein is a method of treating cancer comprising administering a NTRK kinase inhibitor to a subject determined to have a NTRK3 fusion gene.

In some embodiments, the NTRK kinase inhibitor provided herein is a selective TRK inhibitor (e.g., larotrectinib). In other embodiments, the NTRK kinase inhibitor provided herein is a multikinase inhibitor with anti-TRK activity (e.g., entrectinib, TPX-0005/repotrectinib, crizotinib, cabozantinib, altiratinib, foretinib, ponatinib, nintedanib, merestinib, BAY2731954/LOXO-195, MGCD516, PLX7486, DS-6051b and TSR-011). In a specific embodiment, the NTRK kinase inhibitor is larotrectinib (formerly known as LOXO-101, Vitrakviv®). In another specific embodiment, the NTRK kinase inhibitor is entrectinib (formerly known as RXDX-101, Rozlytrek).

The NTRK kinase inhibitor provided herein can be formulated in a pharmaceutical composition which comprises a NTRK kinase inhibitor and a pharmaceutically acceptable excipient. The treatment compound provided herein can be formulated into suitable pharmaceutical compositions for different routes of administration, such as injection, sublingual and buccal, rectal, vaginal, ocular, otic, nasal, inhalation, nebulization, cutaneous, or transdermal. The compounds described above may be formulated into pharmaceutical compositions using techniques and procedures well known in the art (see, e.g., Ansel, Introduction to Pharmaceutical Dosage Forms, (7th ed. 1999)).

The subject administered a therapy provided herein can be a mammal. In one embodiment, the subject is a human. In another embodiment, the subject is a human with cancer.

In some embodiments, the cancer is associated with NTRK fusion. In some embodiments, the cancer is associated with TPM3-NTRK1. In some embodiments, the cancer is associated with LMNA-NTRK1. In some embodiments, the cancer is associated with SOSTM1-NTRK1. In some embodiments, the cancer is associated with TPR-NTRK1. In some embodiments, the cancer is associated with CD74-NTRK1. In some embodiments, the cancer is associated with IRF2BP2-NTRK1. In some embodiments, the cancer is associated with 1MPRIP-NTRK1. In some embodiments, the cancer is associated with RFWD2-NTRK1. In some embodiments, the cancer is associated with TP53-NTRK1. In some embodiments, the cancer is associated with TFG-NTRK1. In some embodiments, the cancer is associated with NFASC-NTRK1. In some embodiments, the cancer is associated with BCAN-NTRK1. In some embodiments, the cancer is associated with MDM4-NTRK1. In some embodiments, the cancer is associated with RABGAP1L-NTRK1. In some embodiments, the cancer is associated with PPL-NTRK1. In some embodiments, the cancer is associated with CHTOP-NTRK1. In some embodiments, the cancer is associated with ARHGEF2-NTRK1. In some embodiments, the cancer is associated with TAF-NTRK1. In some embodiments, the cancer is associated with CEL-NTRK1. In some embodiments, the cancer is associated with SSBP2-NTRK1. In some embodiments, the cancer is associated with GRIPAP1-NTRK1. In some embodiments, the cancer is associated with LRRC71-NTRK1. In some embodiments, the cancer is associated with MRPL24-NTRK1. In some embodiments, the cancer is associated with OKI-NTRK2. In some embodiments, the cancer is associated with NACC2-NTRK2. In some embodiments, the cancer is associated with VCL-NTRK2. In some embodiments, the cancer is associated with AGBL4-NTRK2. In some embodiments, the cancer is associated with PAN3-NTRK2. In some embodiments, the cancer is associated with AFAP1-NTRK2. In some embodiments, the cancer is associated with DAB2IP-NTRK2. In some embodiments, the cancer is associated with TRIM24-NTRK2. In some embodiments, the cancer is associated with SQSTM1-NTRK2. In some embodiments, the cancer is associated with ETV6-NTRK3. In some embodiments, the cancer is associated with BTBD1-NTRK3. In some embodiments, the cancer is associated with EML4-NTRK3. In some embodiments, the cancer is associated with TFG-NTRK3. In some embodiments, the cancer is associated with RBPMS-NTRK3. In some embodiments, the cancer is associated with LYN-NTRK3. In some embodiments, the cancer is associated with NTRK3-HOMER2.

In some embodiments, the cancer is selected from a group consisting of mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, the cancer is a hematological cancer, such as leukemia, lymphoma, or myeloma. In some embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CIVIL), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the cancer is a solid tumor cancer. In some embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer.

In certain embodiments, the cancer is selected from a group consisting of colorectal cancer (CRC), papillary thyroid cancer (PTC), non-small-cell lung carcinoma (NSCLC), sarcoma, pediatric glioma, breast cancer, gallbladder, cholangiocarcinoma, spitzoid melanoma, astrocytoma, glioblastoma (GBM), pancreatic cancer, uterus carcinoma, pilocytic astrocytoma, pediatric glioma, head and neck squamous cell carcinoma (HNSCC), glioma, salivary gland tumor (including acinic cell carcinoma), adult acute myeloid leukemia (AML), nephroma, and inflammatory myofibroblastic tumor (IMT).

Various delivery systems are known and can be used to administer a NTRK inhibitor provided herein, including, but not limited to, parenteral administration (e.g., intradermal, intramuscular, intraperitoneal, intravenous and subcutaneous), epidural, and mucosal (e.g., intranasal and oral routes).

In a specific embodiment, a NTRK inhibitor is administered intranasally, intramuscularly, intravenously, or subcutaneously. In another specific embodiment, a NTRK inhibitor is administered orally. The NTRK inhibitors, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent.

In a specific embodiment, it may be desirable to administer a NTRK inhibitor provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In another embodiment, a NTRK inhibitor, or a composition provided herein can be delivered in a vesicle, in particular a liposome. In another embodiment, a NTRK inhibitor, or a composition provided herein can be delivered in a controlled release or sustained release system.

The amount of a NTRK inhibitor provided herein that will be effective in the prevention and/or treatment of cancer can be determined by standard clinical techniques. In addition, in vitro assays may optionally be employed to help identify optimal dosage ranges. The precise dose to be employed in the formulation will also depend on the route of administration, and the seriousness of a disease or condition, and in some embodiments, should be decided according to the judgment of the practitioner and each patient's circumstances. Effective doses may also be extrapolated from dose-response curves derived from in vitro or animal model test systems.

The dose administered to a mammal, particularly a human, in the context of the present disclosure should be sufficient to affect a therapeutic response in the mammal over a reasonable time frame. One skilled in the art will recognize that dosage will depend upon a variety of factors including the potency of the specific compound, the age, condition and body weight of the patient, as well as the stage/severity of the disease. The dose will also be determined by the route (administration form) timing and frequency of administration.

The NTRK inhibitor can be delivered as a single dose (e.g., a single bolus injection), or over time (e.g., continuous infusion over time or divided bolus doses over time). The agent can be administered repeatedly if necessary, for example, until the patient experiences stable disease or regression, or until the patient experiences disease progression or unacceptable toxicity. Stable disease or lack is determined by methods known in the art such as evaluation of patient symptoms, physical examination, and visualization of the tumor that has been imaged using X-ray, CAT, PET, MRI scan, or other commonly accepted evaluation modalities.

The NTRK inhibitor can be administered once daily (QD) or divided into multiple daily doses such as twice daily (BID), three times daily (TID), and four times daily (QID). In addition, the administration can be continuous (i.e., daily for consecutive days or every day) or intermittent, e.g., in cycles (i.e., including days, weeks, or months of rest without drug). In some embodiments, the frequency of administration is in the range of about a daily dose to about a monthly dose. In certain embodiments, administration is once a day, twice a day, three times a day, four times a day, once every other day, twice a week, once every week, once every two weeks, once every three weeks, or once every four weeks. In certain embodiments, the compound is administered once per day from one day to six months, from one week to three months, from one week to four weeks, from one week to three weeks, or from one week to two weeks.

### 7.6. Kit

In another aspect, provided herein is a kit for performing the methods provided herein, e.g., for detecting a NTRK fusion gene, comprising an agent for detecting a NTRK fusion gene in the sample by RNA in situ hybridization, wherein the agent comprises a probe comprising a nucleic acid sequence complementary to a region of mRNA encoding a kinase domain of NTRK1, NTRK2, or NTRK3.

In some embodiments, the kit provided herein comprises a probe comprising a nucleic acid sequence complementary to a region of mRNA encoding a kinase domain of NTRK1, NTRK2, or NTRK3. In some embodiments, a probe comprising a nucleic acid sequence complementary to a region of mRNA encoding a kinase domain of NTRK1, NTRK2, or NTRK3 comprises plural probes, each of which comprises a region complementary to a region of the mRNA encoding the kinase domain of NTRK1, NTRK2, or NTRK3. As described above, the mRNA encoding the kinase domain of NTRK1 comprises SEQ ID NO:3, the mRNA encoding the kinase domain of NTRK2 comprises SEQ ID NO:10, and the mRNA encoding the kinase domain of NTRK3 comprises SEQ ID NO:17.

In other embodiments, the kit provided herein comprises one or more probes, each of which comprises a region complementary to a region of the mRNA encoding the kinase domain of NTRK1. In other embodiments, the method comprises using one or more probes, each of which comprises a region complementary to a region of the mRNA encoding the kinase domain of NTRK2. In yet other embodiments, the method comprises using one or more probes, each of which comprises a region complementary to a region of the mRNA encoding the kinase domain of NTRK3.

In other embodiments, the kit comprises a probe pool comprising at least two of (a) a first probe complementary to a region of the mRNA encoding the kinase domain of NTRK1; (b) a second probe complementary to a region of the mRNA encoding the kinase domain of NTRK2; and (c) a third probe complementary to a region of the mRNA encoding the kinase domain of NTRK3.

In some embodiments, the probe pool comprises the first probe complementary to a region of the mRNA encoding the kinase domain of NTRK1; and the second probe complementary to a region of the mRNA encoding the kinase domain of NTRK2. In other embodiments, the probe pool comprises the first probe complementary to a region of the mRNA encoding the kinase domain of NTRK1; and the third probe complementary to a region of the mRNA encoding the kinase domain of NTRK3. In yet other embodiments, the probe pool comprises the second probe complementary to a region of the mRNA encoding the kinase domain of NTRK2; and the third probe complementary to a region of the mRNA encoding the kinase domain of NTRK3. In yet other embodiments, the probe pool comprises the first probe complementary to a region of the mRNA encoding the kinase domain of NTRK1; the second probe complementary to a region of the mRNA encoding the kinase domain of NTRK2; and the third probe complementary to a region of the mRNA encoding the kinase domain of NTRK3.

In a specific embodiment, the probe that comprises a region complementary to a region of the mRNA encoding the kinase domain of NTRK1, is complementary to a region of SEQ ID NO:5. In some embodiments, the method comprises use two or more probes complementary to different regions within SEQ ID NO:5. In a specific embodiment, the probe that comprises a region complementary to a region of the mRNA encoding the kinase domain of NTRK1, is complementary to a region SEQ ID NO:6. In some embodiments, the method comprises use two or more probes complementary to different regions within SEQ ID NO:6. In a specific embodiment, the probe that comprises a region complementary to a region of the mRNA encoding the kinase domain of NTRK1, is complementary to a region of SEQ ID NO:7. In some embodiments, the method comprises use two or more probes complementary to different regions within SEQ ID NO:7.

In a specific embodiment, the probe that comprises a region complementary to a region of the mRNA encoding the kinase domain of NTRK2, is complementary to a region of SEQ ID NO:12. In some embodiments, the method comprises use two or more probes complementary to different regions within SEQ ID NO:12. In a specific embodiment, the probe that comprises a region complementary to a region of the mRNA encoding the kinase domain of NTRK2, is complementary to a region of SEQ ID NO:13. In some embodiments, the method comprises use two or more probes complementary to different regions within SEQ ID NO:13. In a specific embodiment, the probe that comprises a region complementary to a region of the mRNA encoding the kinase domain of NTRK2, is complementary to a region of SEQ ID NO:14. In some embodiments, the method comprises use two or more probes complementary to different regions within SEQ ID NO:14.

In a specific embodiment, the probe that comprises a region complementary to a region of the mRNA encoding the kinase domain of NTRK3, is complementary to a region of SEQ ID NO:19. In some embodiments, the method comprises use two or more probes complementary to different regions within SEQ ID NO:19. In a specific embodiment, the probe that comprises a region complementary to a region of the mRNA encoding the kinase domain of NTRK3, is complementary to a region of SEQ ID NO:20. In some embodiments, the method comprises use two or more probes complementary to different regions within SEQ ID NO:20. In a specific embodiment, the probe that comprises a region complementary to a region of the mRNA encoding the kinase domain of NTRK3, is complementary to a region of SEQ ID NO:21. In some embodiments, the method comprises use two or more probes complementary to different regions within SEQ ID NO:21.

In some embodiments, the probes used in the RNA in situ hybridization provided herein comprise plural probes complementary to different regions within SEQ ID NO:5, plural probes complementary to different regions within SEQ ID NO:12, and plural probes complementary to different regions within SEQ ID NO:19.

In some embodiments, the probes used in the RNA in situ hybridization provided herein comprise plural probes complementary to different regions within SEQ ID NO:6, plural probes complementary to different regions within SEQ ID NO:13, and plural probes complementary to different regions within SEQ ID NO:20.

In some embodiments, the probes used in the RNA in situ hybridization provided herein comprise plural probes complementary to different regions within SEQ ID NO:7, plural probes complementary to different regions within SEQ ID NO:14, and plural probes complementary to different regions within SEQ ID NO:21.

In some embodiments, the probes used in the RNA in situ hybridization provided herein comprise plural probes complementary to different regions within a sequence that is selected from the group consisting of SEQ ID NO:3, SEQ ID NO:5, SEQ ID NO:6, and SEQ ID NO:7; plural probes complementary to different regions within a sequence that is selected from the group consisting of SEQ ID NO:10, SEQ ID NO:12, SEQ ID NO:13, and SEQ ID NO:14; and plural probes complementary to different regions within a sequence that is selected from the group consisting of SEQ ID NO:17, SEQ ID NO:19, SEQ ID NO:20, and SEQ ID NO:21.

In a specific embodiment, the kit provided herein comprises agents for performing RNAscope® as described in more detail in, e.g., U.S. Pat. Nos. 7,709,198, 8,604,182, and 8,951,726. In some embodiments, the kit comprises at least one set of two or more target probes capable of hybridizing to a target nucleic acid; a SGC capable of hybridizing to said set of two or more target probes, wherein said SGC comprises a label probe and a nucleic acid component capable of hybridizing to said set of two or more target probes, wherein the target nucleic acid comprises a region of mRNA encoding a kinase domain of NTRK1, NTRK2, or NTRK3.

In some embodiments, the target probes comprises a target (T) section and a label (L) section, wherein the T section is a nucleic acid sequence complementary to a section on the target nucleic acid and the L section is a nucleic acid sequence complementary to a section on the nucleic acid component of the signal generating complex, and wherein the T sections of the two or more target probes are complementary to non-overlapping regions of the target nucleic acid, and the L sections of the two or more target probes are complementary to non-overlapping regions of the nucleic acid component of the generating complex. In some embodiments, in all the two or more target probes the T section is at 5′ of the L section or in all the two or more target probes the T section is at 3′ of the L section.

In some embodiments, the kit further comprises SGC as described in Section 7.3 above, which may incudes label probe, amplifier, pre-amplifier, and/or pre-pre-amplifier.

In some embodiments, the kit further comprises other agents or materials for performing RNA ISH, including fixing agents and agents for treating samples for preparing hybridization, agents for washing samples, and so on.

The kit may further comprise “packaging material” which refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits provided herein can include labels or inserts. Labels or inserts include information on a condition, disorder, disease, or symptom for which the kit component may be used for. Labels or inserts can include instructions for a clinician or for a subject to use one or more of the kit components in a method, treatment protocol, or therapeutic regimen. In some embodiments, labels or inserts include information on cancers for which the kit component may be used for, such as colorectal cancer (CRC), papillary thyroid cancer (PTC), non-small-cell lung carcinoma (NSCLC), sarcoma, pediatric glioma, breast cancer, gallbladder, cholangiocarcinoma, spitzoid melanoma, astrocytoma, glioblastoma (GBM), pancreatic cancer, uterus carcinoma, pilocytic astrocytoma, pediatric glioma, head and neck squamous cell carcinoma (HNSCC), glioma, salivary gland tumor (including acinic cell carcinoma), adult acute myeloid leukemia (AML), nephroma, and inflammatory myofibroblastic tumor (IMT), breast secretory carcinoma, infantile (congenital) fibrosarcoma, mammary analogue secretory carcinoma of salivary glands, congenital mesoblastic nephroma, spitz tumors, intrahepatic cholangiocarcinoma, appendiceal adenocarcinoma, pediatric DIPG and non-brainstem high-grade glioma, uterine sarcoma, thyroid carcinoma, sarcoma (NOS), GIST, lung adenocarcinoma, ph-like acute lymphoblastic leukemia, colon adenocarcinoma, brain low-grade glioma, or breast invasive carcinoma. In some embodiments, labels or inserts include information on cancers for which the kit component may be used for, such as mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof. In some embodiments, labels or inserts include information on cancers for which the kit component may be used for a hematological cancer, such as Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the cancer is chronic lymphocytic leukemia (CLL). In yet another specific embodiment, the cancer is multiple myeloma (MM). In other embodiments, labels or inserts include information on cancers for which the kit component may be used for a solid tumor, such as a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer. In some embodiments, labels or inserts include information on cancers for which the kit component may be used for a cancer that is selected from a group consisting of colorectal cancer (CRC), papillary thyroid cancer (PTC), non-small-cell lung carcinoma (NSCLC), sarcoma, pediatric glioma, breast cancer, gallbladder, cholangiocarcinoma, spitzoid melanoma, astrocytoma, glioblastoma (GBM), pancreatic cancer, uterus carcinoma, pilocytic astrocytoma, pediatric glioma, head and neck squamous cell carcinoma (HNSCC), glioma, salivary gland tumor (including acinic cell carcinoma), adult acute myeloid leukemia (AML), nephroma, and inflammatory myofibroblastic tumor (IMT).

In some embodiments, the labels or inserts include instructions for a clinician to interpret the RNA ISH data produced by the reagents from the kit on a subject, whether and when to employ other detection technology, and subsequently to make a decision on whether to administer a compound, including the ones disclosed in Section 7.5 hereinabove, to the subject. In some embodiments, if RNA ISH produces an unambiguously positive staining produced by the reagents from the kit on a subject, a clinician starts administering a compound, including the ones disclosed in Section 7.5 hereinabove, to the subject. In some embodiments, if RNA ISH produces an unambiguously positive staining produced by the reagents from the kit on a subject, a clinician employs other one or more detection technology for NTRK gene fusion, as disclosed in Section 7.2 hereinabove, with a confirmatory positive result, the clinician starts administering a compound, including the ones disclosed in Section 6.5 hereinabove, to the subject. In some embodiments, if RNA ISH produces an ambiguously positive staining (i.e., weak staining) produced by the reagents from the kit on a subject, a clinician employs other one or more detection technology for NTRK gene fusion, as disclosed in Section 7.2 hereinabove, with a confirmatory positive result, the clinician starts administering a compound, including the ones disclosed in Section 7.5 hereinabove, to the subject. In some embodiments, if RNA ISH produces an ambiguously positive staining (i.e., weak staining) produced by the reagents from the kit on a subject, a clinician employs other one or more detection technology for NTRK gene fusion, as disclosed in Section 7.2 hereinabove, with a negative result from the other detection technologies, especially NGS technology, the clinician does not start administering a compound that targets NTRK to the subject.

Labels or inserts can include “printed matter,” e.g., paper or cardboard, separate or affixed to a component, a kit or packing material (e.g., a box), or attached to, for example, an ampule, tube or vial containing a kit component. Labels or inserts can additionally include a computer readable medium, such as a disk (e.g., hard disk, card, and memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

In some embodiments, the kit provided herein is for determination if a subject has cancer or is likely to develop cancer.

### 8. EXAMPLES

The following is a description of various methods and materials used in the studies, and are put forth so as to provide those of ordinary skill in the art with a complete disclosure and description of how to make and use the present disclosure, and are not intended to limit the scope of what the inventors regard as their disclosure nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present disclosure. Efforts have been made to ensure accuracy with respect to numbers used (e.g., amounts, percentages, etc.), but some experimental errors and deviations should be accounted for.

### 8.1. Example I—Detection of NTRK Gene Fusion by RNAscope® in Comparison with IHC in NTRK Fusion-Positive and NTRK Fusion-Negative Tumor Samples

This example exemplifies the detection of NTRK gene fusion using the present method (e.g., by RNAscope®).

RNA ISH was performed using pan-NTRK probe (Hs-NTRK pool, RNAscope® 2.5 LS, Advanced Cell Diagnostics). Specifically, RNAscope® detection for pan-NTRK (NTRK1 (ACD No. 852778), NTRK2 (ACD No. 852808), and NTRK3 (ACD No. 852838)) was performed on a Leica Biosystems BOND RX research staining robot using the RNAscope® 2.5 LS Reagent Kit-BROWN (ACD No. 322100) per the manufacturer's recommendations. NTRK ISH positivity was graded semi-quantitatively as grade 1, 2, and 3. Grading was performed as follows: Grade 1 for staining conspicuous at 400× magnification, Grade 2 at 100× magnification and Grade 3 at 20× magnification. The results were compared to IHC results using pan-TRK IHC (EPR17341, Abcam).

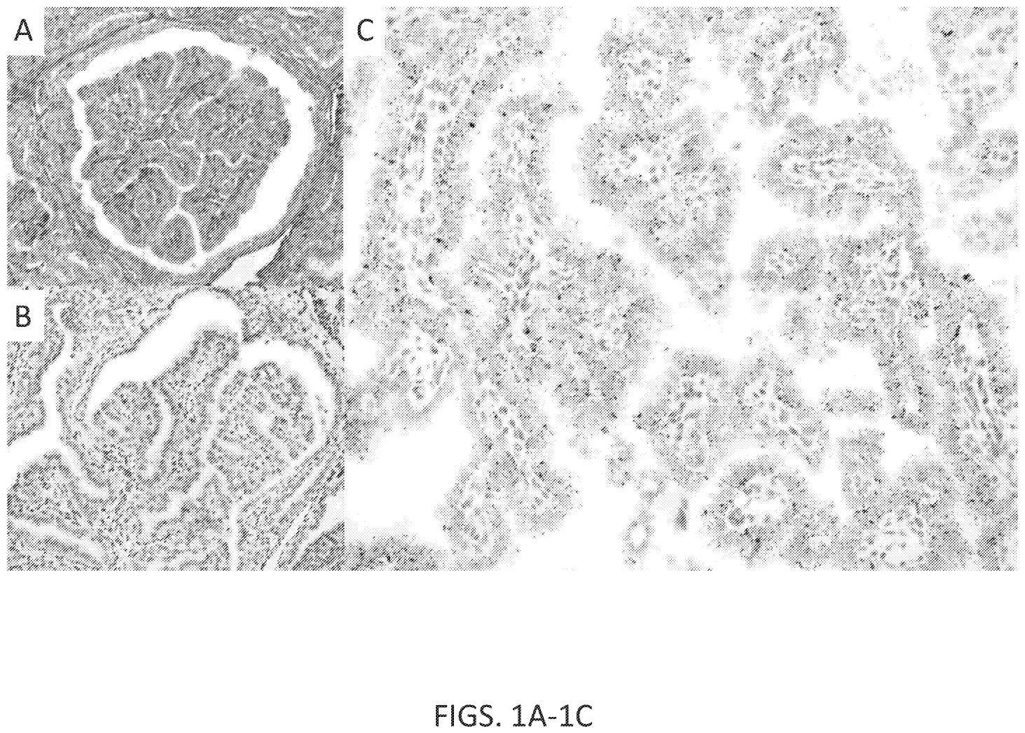

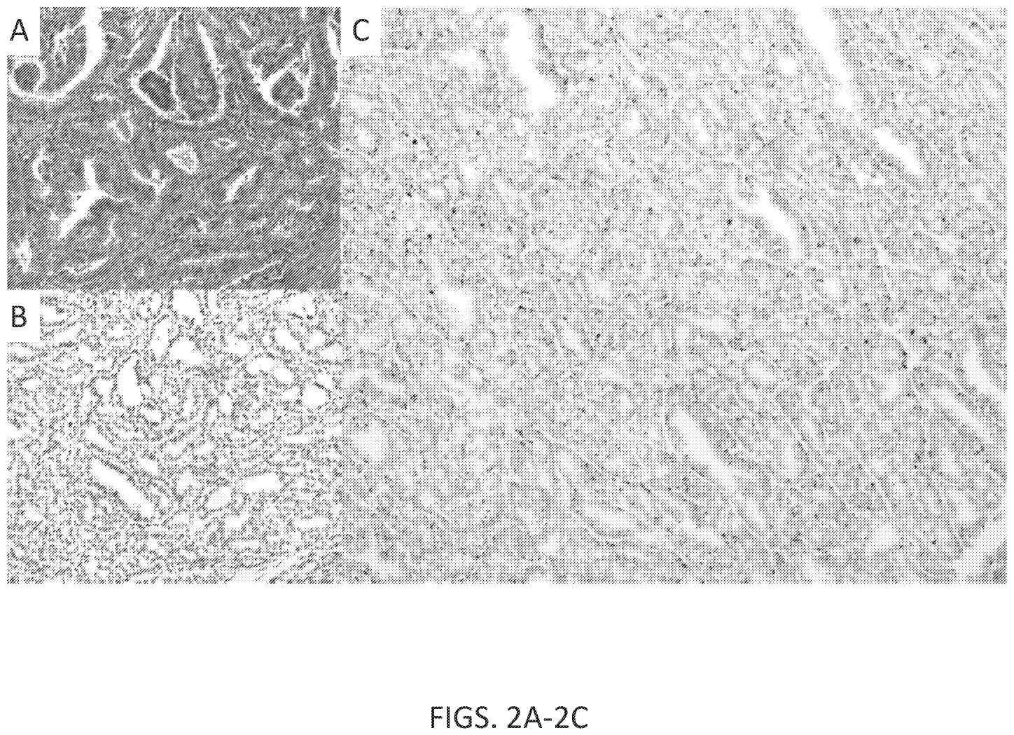

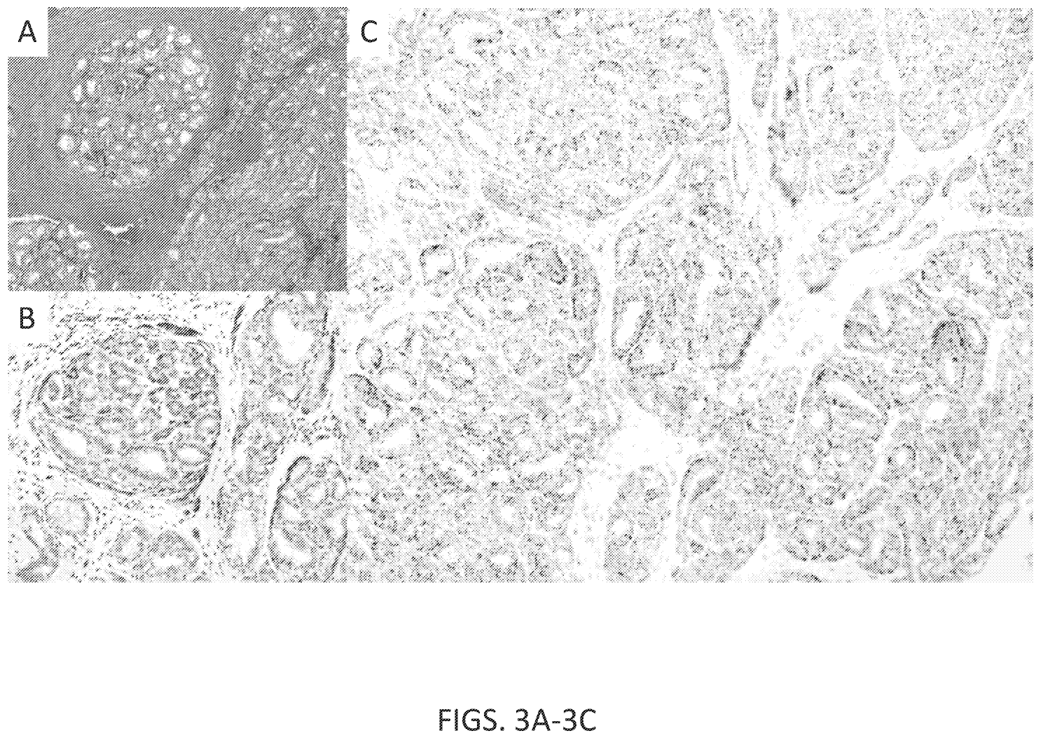

The results in the NTRK fusion-positive tumor samples are shown in Table 1 (below). Additionally, as shown in FIGS. 1-3, representative sample sections were obtained from each patient's thyroid gland (Cases 1-7, 1-6, and 1-1, respectively), and were stained for hematoxylin-eosin (FIGS. 1A, 2A, and 3A), pan-TRK immunohistochemistry (FIGS. 1B, 2B, and 3B), and pan-NTRK in situ hybridization (FIGS. 1C, 2C, and 3C). All eight NTRK fusion-positive tumor samples, including NTRK1, NTRK2, or NTRK3 gene fusion, were positive for pan-NTRK RNA ISH. All eight NTRK fusion-negative tumors were negative for pan-TRK IHC and ISH.

TABLE 1
Comparison between RNA ISH and IHC in the NTRK fusion-positive cases

| Case | | | | | NTRK- | Pan-TRK | Pan-NTRK |
|---|---|---|---|---|---|---|---|
| Number | Diagnosis | Age | Gender | Site | Fusion | IHC | ISH |
| 1-1 | Papillary Thyroid | 67 | F | Thyroid | RBPMS- | Positive | 3+ |
| | Carcinoma | | | Gland | NTRK3 | | |
| 1-2 | Papillary Thyroid | 21 | F | Thyroid | RBPMS- | Positive | 2+ |
| | Carcinoma | | | Gland | NTRK3 | | |
| 1-3 | Secretory | 25 | M | Parotid | ETV6- | Positive | 2+ |
| | Carcinoma | | | Gland | NTRK3 | | |
| 1-4 | Metastatic | 78 | M | Brain | EML4- | Positive | 2+ |
| | Papillary | | | | NTRK3 | | |
| | Thyroid | | | | | | |
| | Carcinoma | | | | | | |
| 1-5 | Low-Grade | 54 | M | Brain | BCAN- | Positive | 3+ |
| | Glioneuronal | | | | NTRK1 | | |
| | Tumor | | | | | | |
| 1-6 | Papillary Thyroid | 74 | F | Thyroid | ETV6- | Positive | 3+ |
| | Carcinoma | | | Gland | NTRK3 | | |
| 1-7 | Papillary Thyroid | 24 | M | Thyroid | TPR- | Positive | 3+ |
| | Carcinoma | | | Gland | NTRK1 | | |
| 1-8 | Low-Grade | 26 | M | Brain | SDCCAG8- | Not | 2+ |
| | Glioneuronal | | | | NTRK2 | Performed | |
| | Tumor | | | | | | |

### 8.2. Example II—NTRK Gene Fusion by RNAscope® in Comparison with IHC in NTRK Associated Mesenchymal Tumors

This example exemplifies the detection of NTRK gene fusion using the present method (e.g., by RNAscope®) in NTRK associated mesenchymal tumors (n=9).

RNA ISH was performed using pan-NTRK probe (Hs-NTRK pool, RNAscopex 2.5 LS, Advanced Cell Diagnostics). Specifically, RNAscope® detection for pan-NTRK (NTRK1 (ACD No. 852778), NTRK2 (ACD No. 852808), and NTRK3 (ACD No. 852838)) was performed on a Leica Biosystems BOND RX research staining robot using the RNAscope® 2.5 LS Reagent Kit-BROWN (ACD No. 322100) per the manufacturer's recommendations. NTRK ISH positivity was graded semi-quantitatively as grade 1, 2, and 3. Grading was performed as follows: Grade 1 for staining conspicuous at 400× magnification, Grade 2 at 100× magnification and Grade 3 at 20× magnification. The results were compared to IHC results using pan-TRK IHC (EPR17341, Abcam).

The results in the NTRK associated mesenchymal tumors are shown in Table 2. All mesenchymal tumors including infantile fibrosarcoma (n=4) and lipofibromatosis-like neural tumor (n=4) were positive for both the pan-TRK IHC and ISH.

TABLE 2
Comparison between RNA ISH and IHC in the NTRK associated mesenchymal tumors

| Case | | | | | NTRK- | Pan-TRK | Pan-NTRK |
|---|---|---|---|---|---|---|---|
| Number | Diagnosis | Age | Gender | Site | Fusion | IHC | ISH |
| 2-1 | Lipofibromatosis- | 17 | M | Thigh | NA | positive | 2+ |
| | Like | | | | | | |
| | Neural Tumor | | | | | | |
| 2-2 | Infantile | 3m | M | Buttock | ETV6- | positive | 3+ |
| | Fibrosarcoma | | | | NTRK3 | | |
| 2-3 | NTRK-Rearranged | 26 | M | Palm | NA | positive | 3+ |
| | Spindle Cell | | | | | | |
| | Neoplasm (NOS) | | | | | | |
| 2-4 | Lipofibromatosis- | 8 | M | Forearm | NA | positive | 2+ |
| | Like | | | | | | |
| | Neural Tumor | | | | | | |
| 2-5 | Infantile | 22d | F | leg | ETV6- | positive | 2+ |
| | Fibrosarcoma | | | | NTRK3 | | |
| 2-6 | Lipofibromatosis- | 6 | M | Abdominal | NA | positive | 2+ |
| | Like | | | wall | | | |
| | Neural Tumor | | | | | | |
| 2-7 | Lipofibromatosis- | 10 | M | Abdominal | NA | positive | 2+ |
| | Like | | | wall | | | |
| | Neural Tumor | | | | | | |
| 2-8 | Infantile | 3m | F | Vertex | ETV6- | positive | 2+ |
| | Fibrosarcoma | | | | NTRK3 | | |
| 2-9 | Infantile | 7d | F | Paraspinal | ETV6- | positive | 3+ |
| | Fibrosarcoma | | | | NTRK3 | | |

### 8.3. Example III—NTRK Gene Fusion by RNAscope® in Comparison with IHC in Synovial Sarcoma

This example exemplifies the detection of NTRK gene fusion by the present method (e.g., by RNAscope®) in synovial sarcoma cases (n=83).

RNA ISH was performed using pan-NTRK probe (Hs-NTRK pool, RNAscope® 2.5 LS, Advanced Cell Diagnostics). Specifically, RNAscope® detection for pan-NTRK (NTRK1 (ACD No. 852778), NTRK2 (ACD No. 852808), and NTRK3 (ACD No. 852838)) was performed on a Leica Biosystems BOND RX research staining robot using the RNAscope® 2.5 LS Reagent Kit-BROWN (ACD No. 322100) per the manufacturer's recommendations. NTRK ISH positivity was graded semi-quantitatively as grade 1, 2, and 3. Grading was performed as follows: Grade 1 for staining conspicuous at 400× magnification, Grade 2 at 100× magnification and Grade 3 at 20× magnification. The results were compared to IHC results using pan-TRK IHC (EPR17341, Abcam).

Two synovial sarcoma cases (2.4% of the cases) were identified to be positive for both pan-NTRK ISH and pan-TRK IHC.

These results also suggest that pan-NTRK ISH could serve as a marker of NTRK fusions and may have a role in cases in which IHC results are ambiguous. In addition, combined use of pan-TRK ISH and IHC could also be used to screen for NTRK fusion positive neoplasms.

What is claimed is:

1. A method of detecting a neurotrophic tyrosine receptor kinase (NTRK) fusion gene, comprising:
(i) obtaining a sample; and
(ii) detecting a NTRK fusion gene in the sample by RNA in situ hybridization using a probe comprising a nucleic acid sequence complementary to a region of mRNA encoding a kinase domain of NTRK1, NTRK2, or NTRK3.

2. The method of claim 1, wherein the probe comprises a nucleic acid sequence complementary to a region of the mRNA encoding the kinase domain of NTRK1.

3. The method of claim 2, wherein the probe comprises a nucleic acid sequence complementary to a region within SEQ ID NO:3, SEQ ID NO:5, SEQ ID NO:6, or SEQ ID NO:7.

4. The method of claim 1, wherein the probe comprises a nucleic acid sequence complementary to a region of the mRNA encoding the kinase domain of NTRK2.

5. The method of claim 4, wherein the probe comprises a nucleic acid sequence complementary to a region within SEQ ID NO:10, SEQ ID NO:12, SEQ ID NO:13 or SEQ ID NO:14.

6. The method of claim 1, wherein the probe comprises a nucleic acid sequence complementary to a region of the mRNA encoding the kinase domain of NTRK3.

7. The method of claim 6, wherein the probe comprises a nucleic acid sequence complementary to a region within SEQ ID NO:17, SEQ ID NO:19, SEQ ID NO:20 or SEQ ID NO:21.

8. The method of claim 1, wherein the method comprises detecting a NTRK fusion gene in the sample by RNA in situ hybridization using a probe pool comprising at least two of (a) a first probe comprising a nucleic acid sequence complementary to a region of the mRNA encoding the kinase domain of NTRK1; (b) a second probe comprising a nucleic acid sequence complementary to a region of the mRNA encoding the kinase domain of NTRK2; and (c) a third probe comprising a nucleic acid sequence complementary to a region of the mRNA encoding the kinase domain of NTRK3.

9. The method of claim 8, wherein the probe pool comprises the first probe comprising a nucleic acid sequence complementary to a region of the mRNA encoding the kinase domain of NTRK1; and the second probe comprising a nucleic acid sequence complementary to a region of the mRNA encoding the kinase domain of NTRK2.

10. The method of claim 8, wherein the probe pool comprises the first probe comprising a nucleic acid sequence complementary to a region of the mRNA encoding the kinase domain of NTRK1; and the third probe comprising a nucleic acid sequence complementary to a region of the mRNA encoding the kinase domain of NTRK3.

11. The method of claim 8, wherein the probe pool comprises the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3.

12. The method of claim 8, wherein the probe pool comprises the first probe comprising a nucleic acid sequence complementary to a region of the mRNA encoding the kinase domain of NTRK1; the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3.

13. The method of any one of claims 8 to 12, wherein the first probe comprises a nucleic acid sequence complementary to a region within SEQ ID NO:3, SEQ ID NO:5, SEQ ID NO:6, or SEQ ID NO:7, the second probe comprises a nucleic acid sequence complementary to a region within SEQ ID NO:10, SEQ ID NO:12, SEQ ID NO:13, or SEQ ID NO:14, and the third probe comprises a nucleic acid sequence complementary to a region within SEQ ID NO:17, SEQ ID NO:19, SEQ ID NO:20, or SEQ ID NO:21.

14. The method of any one of claims 1 to 13, wherein the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, SQSTM1-NTRK2, ETV6-NTRK3, BTBD1-NTRK3, EML4-NTRK3, TFG-NTRK3, RBPMS-NTRK3, LYN-NTRK3, and NTRK3-HOMER2.

15. The method of any one of claims 1 to 13, wherein the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, and MRPL24-NTRK1.

16. The method of any one of claims 1 to 13, wherein the NTRK fusion is selected from a group consisting of OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, and SQSTM1-NTRK2.

17. The method of any one of claims 1 to 13, wherein the NTRK fusion is selected from a group consisting of ETV6-NTRK3, BTBD1-NTRK3, EML4-NTRK3, TFG-NTRK3, RBPMS-NTRK3, LYN-NTRK3, and NTRK3-HOMER2.

18. A method of determining if a subject has cancer or is likely to develop cancer, comprising:
(i) obtaining a sample from the subject; and
(ii) detecting a NTRK fusion gene in the sample by RNA in situ hybridization using a probe comprising a nucleic acid sequence complementary to a region of mRNA encoding a kinase domain of NTRK1, NTRK2, or NTRK3.

19. The method of claim 18, wherein the probe comprises a nucleic acid sequence complementary to a region of the mRNA encoding the kinase domain of NTRK1.

20. The method of claim 17, wherein the probe comprises a nucleic acid sequence complementary to a region within SEQ ID NO:3, SEQ ID NO:5, SEQ ID NO:6, or SEQ ID NO:7.

21. The method of claim 18, wherein the probe comprises a nucleic acid sequence complementary to a region of the mRNA encoding the kinase domain of NTRK2.

22. The method of claim 21, wherein the probe comprises a nucleic acid sequence complementary to a region within SEQ ID NO:10, SEQ ID NO:12, SEQ ID NO:13 or SEQ ID NO:14.

23. The method of claim 18, wherein the probe comprises a nucleic acid sequence complementary to a region of the mRNA encoding the kinase domain of NTRK3.

24. The method of claim 23, wherein the probe comprises a nucleic acid sequence complementary to a region within SEQ ID NO:17, SEQ ID NO:19, SEQ ID NO:20 or SEQ ID NO:21.

25. The method of claim 18, wherein the method comprises detecting a NTRK fusion gene in the sample by RNA in situ hybridization using a probe pool comprising at least two of (a) a first probe comprising a nucleic acid sequence complementary to a region of the mRNA encoding the kinase domain of NTRK1; (b) a second probe comprising a nucleic acid sequence complementary to a region of the mRNA encoding the kinase domain of NTRK2; and (c) a third probe comprising a nucleic acid sequence complementary to a region of the mRNA encoding the kinase domain of NTRK3.

26. The method of claim 25, wherein the probe pool comprises the first probe comprising a nucleic acid sequence complementary to a region of the mRNA encoding the kinase domain of NTRK1; and the second probe comprising a nucleic acid sequence complementary to a region of the mRNA encoding the kinase domain of NTRK2.

27. The method of claim 25, wherein the probe pool comprises the first probe comprising a nucleic acid sequence complementary to a region of the mRNA encoding the kinase domain of NTRK1; and the third probe comprising a nucleic acid sequence complementary to a region of the mRNA encoding the kinase domain of NTRK3.

28. The method of claim 25, wherein the probe pool comprises the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3.

29. The method of claim 25, wherein the probe pool comprises the first probe comprising a nucleic acid sequence complementary to a region of the mRNA encoding the kinase domain of NTRK1; the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3.

30. The method of any one of claims 18 to 29, wherein the first probe comprises a nucleic acid sequence complementary to a region within SEQ ID NO:3, SEQ ID NO:5, SEQ ID NO:6, or SEQ ID NO:7, the second probe comprises a nucleic acid sequence complementary to a region within SEQ ID NO:10, SEQ ID NO:12, SEQ ID NO:13, or SEQ ID NO:14, and the third probe comprises a nucleic acid sequence complementary to a region within SEQ ID NO:17, SEQ ID NO:19, SEQ ID NO:20, or SEQ ID NO:21.

31. The method of any one of claims 18 to 30, wherein the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, SQSTM1-NTRK2, ETV6-NTRK3, BTBD1-NTRK3, EML4-NTRK3, TFG-NTRK3, RBPMS-NTRK3, LYN-NTRK3, and NTRK3-HOMER2.

32. The method of any one of claims 18 to 30, wherein the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, and MRPL24-NTRK1.

33. The method of any one of claims 18 to 30, wherein the NTRK fusion is selected from a group consisting of OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, and SQSTM1-NTRK2.

34. The method of any one of claims 18 to 30, wherein the NTRK fusion is selected from a group consisting of ETV6-NTRK3, BTBD1-NTRK3, EML4-NTRK3, TFG-NTRK3, RBPMS-NTRK3, LYN-NTRK3, and NTRK3-HOMER2.

35. The method of any one of claims 18 to 34, wherein the cancer is associated with NTRK fusion.

36. The method of any one of claims 18 to 34, wherein the cancer is selected from a group consisting of colorectal cancer (CRC), papillary thyroid cancer (PTC), non-small-cell lung carcinoma (NSCLC), sarcoma, pediatric glioma, breast cancer, gallbladder, cholangiocarcinoma, spitzoid melanoma, astrocytoma, glioblastoma (GBM), pancreatic cancer, uterus carcinoma, pilocytic astrocytoma, pediatric glioma, head and neck squamous cell carcinoma (HNSCC), glioma, salivary gland tumor (including acinic cell carcinoma), adult acute myeloid leukemia (AML), nephroma, and inflammatory myofibroblastic tumor (IMT).

37. The method of any one of claims 18 to 36, wherein the presence of the NTRK fusion gene indicates that the subject has cancer or is likely to develop cancer.

38. The method of any one of claims 18 to 36, wherein the method further comprises administering a treatment compound to the subject in whom the NTRK fusion gene is detected.

39. The method of claim 38, wherein the treatment compound is a NTRK kinase inhibitor.

40. The method of claim 38, wherein the treatment compound is selected from a group consisting of larotrectinib, entrectinib, TPX-0005/repotrectinib, crizotinib, cabozantinib, altiratinib, foretinib, ponatinib, nintedanib, merestinib, BAY2731954 (LOXO-195), MGCD516, PLX7486, DS-6051b, and TSR-011.

41. The method of claim 38, wherein the treatment compound is larotrectinib or entrectinib.

42. A method of treating cancer comprising administering a NTRK kinase inhibitor to a subject determined to have a NTRK fusion gene using a method comprising:
(i) obtaining a sample from the subject; and
(ii) detecting a NTRK fusion gene in the sample by RNA in situ hybridization using a probe comprising a nucleic acid sequence complementary to a region of mRNA encoding a kinase domain of NTRK1, NTRK2, or NTRK3.

43. The method of claim 42, wherein the probe comprises a nucleic acid sequence complementary to a region of the mRNA encoding the kinase domain of NTRK1.

44. The method of claim 43, wherein the probe comprises a nucleic acid sequence complementary to a region within SEQ ID NO:3, SEQ ID NO:5, SEQ ID NO:6, or SEQ ID NO:7.

45. The method of claim 42, wherein the probe comprises a nucleic acid sequence complementary to a region of the mRNA encoding the kinase domain of NTRK2.

46. The method of claim 45, wherein the probe comprises a nucleic acid sequence complementary to a region within SEQ ID NO:10, SEQ ID NO:12, SEQ ID NO:13 or SEQ ID NO:14.

47. The method of claim 42, wherein the probe comprises a nucleic acid sequence complementary to a region of the mRNA encoding the kinase domain of NTRK3.

48. The method of claim 47, wherein the probe comprises a nucleic acid sequence complementary to a region within SEQ ID NO:17, SEQ ID NO:19, SEQ ID NO:20 or SEQ ID NO:21.

49. The method of claim 42, wherein the method comprises detecting a NTRK fusion gene in the sample by RNA in situ hybridization using a probe pool comprising at least two of (a) a first probe comprising a nucleic acid sequence complementary to a region of the mRNA encoding the kinase domain of NTRK1; (b) a second probe comprising a nucleic acid sequence complementary to a region of the mRNA encoding the kinase domain of NTRK2; and (c) a third probe comprising a nucleic acid sequence complementary to a region of the mRNA encoding the kinase domain of NTRK3.

50. The method of claim 49, wherein the probe pool comprises the first probe comprising a nucleic acid sequence complementary to a region of the mRNA encoding the kinase domain of NTRK1; and the second probe comprising a nucleic acid sequence complementary to a region of the mRNA encoding the kinase domain of NTRK2.

51. The method of claim 49, wherein the probe pool comprises the first probe comprising a nucleic acid sequence complementary to a region of the mRNA encoding the kinase domain of NTRK1; and the third probe comprising a nucleic acid sequence complementary to a region of the mRNA encoding the kinase domain of NTRK3.

52. The method of claim 49, wherein the probe pool comprises the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3.

53. The method of claim 49, wherein the probe pool comprises the first probe comprising a nucleic acid sequence complementary to a region of the mRNA encoding the kinase domain of NTRK1; the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3.

54. The method of any one of claims 42 to 53, wherein the first probe comprises a nucleic acid sequence complementary to a region within SEQ ID NO:3, SEQ ID NO:5, SEQ ID NO:6, or SEQ ID NO:7, the second probe comprises a nucleic acid sequence complementary to a region within SEQ ID NO:10, SEQ ID NO:12, SEQ ID NO:13, or SEQ ID NO:14, and the third probe comprises a nucleic acid sequence complementary to a region within SEQ ID NO:17, SEQ ID NO:19, SEQ ID NO:20, or SEQ ID NO:21.

55. The method of any one of claims 49 to 54, wherein the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, SQSTM1-NTRK2, ETV6-NTRK3, BTBD1-NTRK3, EML4-NTRK3, TFG-NTRK3, RBPMS-NTRK3, LYN-NTRK3, and NTRK3-HOMER2.

56. The method of any one of claims 49 to 54, wherein the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, and MRPL24-NTRK1.

57. The method of any one of claims 49 to 54, wherein the NTRK fusion is selected from a group consisting of OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, and SQSTM1-NTRK2.

58. The method of any one of claims 49 to 54, wherein the NTRK fusion is selected from a group consisting of ETV6-NTRK3, BTBD1-NTRK3, EML4-NTRK3, TFG-NTRK3, RBPMS-NTRK3, LYN-NTRK3, and NTRK3-HOMER2.

59. The method of any one of claims 42 to 58, wherein the cancer is associated with NTRK fusion.

60. The method of any one of claims 42 to 58, wherein the cancer is selected from a group consisting of wherein the cancer is selected from a group consisting of colorectal cancer (CRC), papillary thyroid cancer (PTC), non-small-cell lung carcinoma (NSCLC), sarcoma, pediatric glioma, breast cancer, gallbladder cancer, cholangiocarcinoma, spitzoid melanoma, astrocytoma, glioblastoma (GBM), pancreatic cancer, uterus carcinoma, pilocytic astrocytoma, pediatric glioma, head and neck squamous cell carcinoma (HNSCC), glioma, salivary gland tumor (including acinic cell carcinoma), adult acute myeloid leukemia (AML), nephroma, and inflammatory myofibroblastic tumor (IMT).

61. The method of any one of claims 42 to 60, wherein the presence of the NTRK fusion gene indicates that the subject has cancer or is likely to develop cancer.

62. The method of any one of claims 42 to 61, wherein the treatment compound is a NTRK kinase inhibitor.

63. The method of claim 62, wherein the treatment compound is selected from a group consisting of larotrectinib, entrectinib, TPX-0005/repotrectinib, crizotinib, cabozantinib, altiratinib, foretinib, ponatinib, nintedanib, merestinib, BAY2731954 (LOXO-195), MGCD516, PLX7486, DS-6051b, and TSR-011.

64. The method of claim 63, wherein the treatment compound is larotrectinib.

65. The method of claim 63, wherein the treatment compound is entrectinib.

66. The method of any one of claims 1 to 65, wherein the sample is a tissue specimen or is derived from a tissue specimen.

67. The method of any one of claims 1 to 65, wherein the sample is a blood sample or is derived from a blood sample.

68. The method of any one of claims 1 to 65, wherein the sample is a cytological sample or is derived from a cytological sample.

69. The method of any one of claims 1 to 65, wherein the sample is a tumor sample.

70. The method of any one of claims 1 to 69, wherein the sample is processed for RNA in situ hybridization, wherein optionally the sample is paraffin embedded and/or formalin fixed, wherein optionally the tissue specimen is fresh frozen, wherein optionally the tissue specimen is prepared with a fixative other than formalin, and wherein optionally the fixative other than formalin is selected from the group consisting of ethanol, methanol, Bouin's, B5, and I.B.F.

71. The method of any one of claims 1 to 70, wherein the probe is entirely complementary to the region of mRNA encoding a kinase domain of NTRK1, NTRK2, or NTRK3.

72. The method of any one of claims 1 to 70, wherein the probe further comprises a nucleic acid sequence not complementary to the region of mRNA encoding a kinase domain of NTRK1, NTRK2, or NTRK3.

73. The method of claim 72, wherein a 3′ region of the probe is complementary to the region of mRNA encoding a kinase domain of NTRK1, NTRK2, or NTRK3.

74. The method of claim 72, wherein a 5′ region of the probe is complementary to the region of mRNA encoding a kinase domain of NTRK1, NTRK2, or NTRK3.

75. A kit for detecting a neurotrophic tyrosine receptor kinase (NTRK) fusion gene, comprising an agent for detecting a NTRK fusion gene in the sample by RNA in situ hybridization, wherein the agent comprises a probe comprising a nucleic acid sequence complementary to a region of mRNA encoding a kinase domain of NTRK1, NTRK2, or NTRK3.

76. The kit of claim 75, wherein the probe comprises a nucleic acid sequence complementary to a region of the mRNA encoding the kinase domain of NTRK1.

77. The kit of claim 76, wherein the probe comprises a nucleic acid sequence complementary to a region within SEQ ID NO:3, SEQ ID NO:5, SEQ ID NO:6, or SEQ ID NO:7.

78. The kit of claim 75, wherein the probe comprises a nucleic acid sequence complementary to a region of the mRNA encoding the kinase domain of NTRK2.

79. The kit of claim 78, wherein the probe comprises a nucleic acid sequence complementary to a region within SEQ ID NO:10, SEQ ID NO:12, SEQ ID NO:13 or SEQ ID NO:14.

80. The kit of claim 75, wherein the probe comprises a nucleic acid sequence complementary to a region of the mRNA encoding the kinase domain of NTRK3.

81. The kit of claim 80, wherein the probe comprises a nucleic acid sequence complementary to a region within SEQ ID NO:17, SEQ ID NO:19, SEQ ID NO:20 or SEQ ID NO:21.

82. The kit of claim 75, wherein the kit comprises a probe pool comprising at least two of (a) a first probe comprising a nucleic acid sequence complementary to a region of the mRNA encoding the kinase domain of NTRK1; (b) a second probe comprising a nucleic acid sequence complementary to a region of the mRNA encoding the kinase domain of NTRK2; and (c) a third probe comprising a nucleic acid sequence complementary to a region of the mRNA encoding the kinase domain of NTRK3.

83. The kit of claim 82, wherein the probe pool comprises the first probe comprising a nucleic acid sequence complementary to a region of the mRNA encoding the kinase domain of NTRK1; and the second probe comprising a nucleic acid sequence complementary to a region of the mRNA encoding the kinase domain of NTRK2.

84. The kit of claim 82, wherein the probe pool comprises the first probe comprising a nucleic acid sequence complementary to a region of the mRNA encoding the kinase domain of NTRK1; and the third probe comprising a nucleic acid sequence complementary to a region of the mRNA encoding the kinase domain of NTRK3.

85. The kit of claim 82, wherein the probe pool comprises the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3.

86. The kit of claim 82, wherein the probe pool comprises the first probe comprising a nucleic acid sequence complementary to a region of the mRNA encoding the kinase domain of NTRK1; the second probe comprising a nucleic acid sequence complementary to a region of the mRNA encoding the kinase domain of NTRK2; and the third probe comprising a nucleic acid sequence complementary to a region of the mRNA encoding the kinase domain of NTRK3.

87. The kit of any one of claims 75 to 86, wherein the first probe comprises a nucleic acid sequence complementary to a region within SEQ ID NO:3, SEQ ID NO:5, SEQ ID NO:6, or SEQ ID NO:7, the second probe comprises a nucleic acid sequence complementary to a region within SEQ ID NO:10, SEQ ID NO:12, SEQ ID NO:13, or SEQ ID NO:14, and the third probe comprises a nucleic acid sequence complementary to a region within SEQ ID NO:17, SEQ ID NO:19, SEQ ID NO:20, or SEQ ID NO:21.

88. The kit of any one of claims 75 to 87, wherein the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, MRPL24-NTRK1, OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, SQSTM1-NTRK2, ETV6-NTRK3, BTBD1-NTRK3, EML4-NTRK3, TFG-NTRK3, RBPMS-NTRK3, LYN-NTRK3, and NTRK3-HOMER2.

89. The kit of any one of claims 75 to 87, wherein the NTRK fusion is selected from a group consisting of TPM3-NTRK1, LMNA-NTRK1, SOSTM1-NTRK1, TPR-NTRK1, CD74-NTRK1, IRF2BP2-NTRK1, MPRIP-NTRK1, RFWD2-NTRK1, TP53-NTRK1, TFG-NTRK1, NFASC-NTRK1, BCAN-NTRK1, MDM4-NTRK1, RABGAP1L-NTRK1, PPL-NTRK1, CHTOP-NTRK1, ARHGEF2-NTRK1, TAF-NTRK1, CEL-NTRK1, SSBP2-NTRK1, GRIPAP1-NTRK1, LRRC71-NTRK1, and MRPL24-NTRK1.

90. The kit of any one of claims 75 to 87, wherein the NTRK fusion is selected from a group consisting of OKI-NTRK2, NACC2-NTRK2, VCL-NTRK2, AGBL4-NTRK2, PAN3-NTRK2, AFAP1-NTRK2, DAB2IP-NTRK2, TRIM24-NTRK2, and SQSTM1-NTRK2.

91. The kit of any one of claims 75 to 87, wherein the NTRK fusion is selected from a group consisting of ETV6-NTRK3, BTBD1-NTRK3, EML4-NTRK3, TFG-NTRK3, RBPMS-NTRK3, LYN-NTRK3, and NTRK3-HOMER2.

92. The kit of any one of claims 75 to 91, wherein the probe is entirely complementary to the region of mRNA encoding a kinase domain of NTRK1, NTRK2, or NTRK3.

93. The kit of any one of claims 75 to 91, wherein the probe further comprises a nucleic acid sequence not complementary to the region of mRNA encoding a kinase domain of NTRK1, NTRK2, or NTRK3.

94. The kit of claim 93, wherein a 3′ region of the probe is complementary to the region of mRNA encoding a kinase domain of NTRK1, NTRK2, or NTRK3.

95. The kit of claim 93, wherein a 5′ region of the probe is complementary to the region of mRNA encoding a kinase domain of NTRK1, NTRK2, or NTRK3.


SEQUENCE LISTING (21 sequences)

SEQ ID NO: 1 - DNA, 2647 bp, Artificial Sequence
  Synthetic
tgcagctgggagcgcacagacggctgccccgcctgagcgaggcgggcgccgccgcgatgctgcgaggcggacggcgcgggcagcttggctggcacagctgggctgcggggccgggcagcctgctggcttggctgatactggcatctgcgggcgccgcaccctgccccgatgcctgctgcccccacggctcctcgggactgcgatgcacccgggatggggccctggatagcctccaccacctgcccggcgcagagaacctgactgagctctacatcgagaaccagcagcatctgcagcatctggagctccgtgatctgaggggcctgggggagctgagaaacctcaccatcgtgaagagtggtctccgtttcgtggcgccagatgccttccatttcactcctcggctcagtcgcctgaatctctccttcaacgctctggagtctctctcctggaaaactgtgcagggcctctccttacaggaactggtcctgtcggggaaccctctgcactgttcttgtgccctgcgctggctacagcgctgggaggaggagggactgggcggagtgcctgaacagaagctgcagtgtcatgggcaagggcccctggcccacatgcccaatgccagctgtggtgtgcccacgctgaaggtccaggtgcccaatgcctcggtggatgtgggggacgacgtgctgctgcggtgccaggtggaggggcggggcctggagcaggccggctggatcctcacagagctggagcagtcagccacggtgatgaaatctgggggtctgccatccctggggctgaccctggccaatgtcaccagtgacctcaacaggaagaacgtgacgtgctgggcagagaacgatgtgggccgggcagaggtctctgttcaggtcaacgtctccttcccggccagtgtgcagctgcacacggcggtggagatgcaccactggtgcatccccttctctgtggatgggcagccggcaccgtctctgcgctggctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagcaattatatttattatcccttgaaaaaaaaaa

SEQ ID NO: 2 - protein, 790 aa, Artificial Sequence
  Synthetic
MLRGGRRGQLGWHSWAAGPGSLLAWLILASAGAAPCPDACCPHGSSGLRCTRDGALDSLHHLPGAENLTELYIENQQHLQHLELRDLRGLGELRNLTIVKSGLRFVAPDAFHFTPRLSRLNLSFNALESLSWKTVQGLSLQELVLSGNPLHCSCALRWLQRWEEEGLGGVPEQKLQCHGQGPLAHMPNASCGVPTLKVQVPNASVDVGDDVLLRCQVEGRGLEQAGWILTELEQSATVMKSGGLPSLGLTLANVTSDLNRKNVTCWAENDVGRAEVSVQVNVSFPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPDTNSTSGDPVEKKDETPFGVSVAVGLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 3 - DNA, 813 bp, Artificial Sequence
  Synthetic
atcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccgg

SEQ ID NO: 4 - protein, 271 aa, Artificial Sequence
  Synthetic
IVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHAR

SEQ ID NO: 5 - DNA, 949 bp, Artificial Sequence
  Synthetic
aggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagcaattatatttatt

SEQ ID NO: 6 - DNA, 1616 bp, Artificial Sequence
  Synthetic
gctcttcaatggctccgtgctcaatgagaccagcttcatcttcactgagttcctggagccggcagccaatgagaccgtgcggcacgggtgtctgcgcctcaaccagcccacccacgtcaacaacggcaactacacgctgctggctgccaaccccttcggccaggcctccgcctccatcatggctgccttcatggacaaccctttcgagttcaaccccgaggaccccatccctgacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagc

SEQ ID NO: 7 - DNA, 1516 bp, Artificial Sequence
  Synthetic
aaccagcccacccacgtcaacaacggcaactacacgctgctggctgccaaccccttcggccaggcctccgcctccatcatggctgccttcatggacaaccctttcgagttcaaccccgaggaccccatccctgacactaacagcacatctggagacccggtggagaagaaggacgaaacaccttttggggtctcggtggctgtgggcctggccgtctttgcctgcctcttcctttctacgctgctccttgtgctcaacaaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctagggggccggcccaggggctgggagtggttagccggaatactggggcctgccctcagcatcccccatagctcccagcagccccagggtgatctcaaagtatctaattcaccctcagcatgtgggaagggacaggtgggggctgggagtagaggatgttcctgcttctctaggcaaggtcccgtcatagc

SEQ ID NO: 8 - DNA, 9205 bp, Artificial Sequence
  Synthetic
accagagccctcggaagtgtcaggcactgcggtgtattttccccgttctcttttaaatgactgcggaaaaacagattccgagccgcaaaagggaagacggattctcagacaaggcttgcaaatgccccgcagccatcatttaactgcacccgcagaatagttacggtttgtcacccgaccctcccggatcgcctaatttgtccctagtgagaccccgaggctctgcccgcgcctggcttcttcgtagctggatgcatatcgtgctccgggcagcgcgggcgcagggcacgcgttcgcgcacaccctagcacacatgaacacgcgcaagagctgaaccaagcacggtttccatttcaaaaagggagacagcctctaccgcgattgtagaagagactgtggtgtgaattagggaccgggaggcgtcgaacggaggaacggttcatcttagagactaattttctggagtttctgcccctgctctgcgtcagccctcacgtcacttcgccagcagtagcagaggcggcggcggcggctcccggaattgggttggagcaggagcctcgctggctgcttcgctcgcgctctacgcgctcagtccccggcggtagcaggagcctggacccaggcgccgccggcgggcgtgaggcgccggagcccggcctcgaggtgcataccggacccccattcgcatctaacaaggaatctgcgccccagagagtcccgggagcgccgccggtcggtgcccggcgcgccgggccatgcagcgacggccgccgcggagctccgagcagcggtagcgcccccctgtaaagcggttcgctatgccggggccactgtgaaccctgccgcctgccggaacactcttcgctccggaccagctcagcctctgataagctggactcggcacgcccgcaacaagcaccgaggagttaagagagccgcaagcgcagggaaggcctccccgcacgggtgggggaaagcggccggtgcagcgcggggacaggcactcgggctggcactggctgctagggatgtcgtcctggataaggtggcatggacccgccatggcgcggctctggggcttctgctggctggttgtgggcttctggagggccgctttcgcctgtcccacgtcctgcaaatgcagtgcctctcggatctggtgcagcgacccttctcctggcatcgtggcatttccgagattggagcctaacagtgtagatcctgagaacatcaccgaaattttcatcgcaaaccagaaaaggttagaaatcatcaacgaagatgatgttgaagcttatgtgggactgagaaatctgacaattgtggattctggattaaaatttgtggctcataaagcatttctgaaaaacagcaacctgcagcacatcaattttacccgaaacaaactgacgagtttgtctaggaaacatttccgtcaccttgacttgtctgaactgatcctggtgggcaatccatttacatgctcctgtgacattatgtggatcaagactctccaagaggctaaatccagtccagacactcaggatttgtactgcctgaatgaaagcagcaagaatattcccctggcaaacctgcagatacccaattgtggtttgccatctgcaaatctggccgcacctaacctcactgtggaggaaggaaagtctatcacattatcctgtagtgtggcaggtgatccggttcctaatatgtattgggatgttggtaacctggtttccaaacatatgaatgaaacaagccacacacagggctccttaaggataactaacatttcatccgatgacagtgggaagcagatctcttgtgtggcggaaaatcttgtaggagaagatcaagattctgtcaacctcactgtgcattttgcaccaactatcacatttctcgaatctccaacctcagaccaccactggtgcattccattcactgtgaaaggcaaccccaaaccagcgcttcagtggttctataacggggcaatattgaatgagtccaaatacatctgtactaaaatacatgttaccaatcacacggagtaccacggctgcctccagctggataatcccactcacatgaacaatggggactacactctaatagccaagaatgagtatgggaaggatgagaaacagatttctgctcacttcatgggctggcctggaattgacgatggtgcaaacccaaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaagatttctcatggtttggatttgggaaagtaaaatcaagacaaggtgttggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctagggcccttttccccagaccgatccttcccaacgtactcctcagacgggctgagaggatgaacatcttttaactgccgctggaggccaccaagctgctctccttcactctgacagtattaacatcaaagactccgagaagctctcgagggaagcagtgtgtacttcttcatccatagacacagtattgacttctttttggcattatctctttctctctttccatctcccttggttgttcctttttctttttttaaattttctttttcttttttttttcgtcttccctgcttcacgattcttaccctttcttttgaatcaatctggcttctgcattactattaactctgcatagacaaaggccttaacaaacgtaatttgttatatcagcagacactccagtttgcccaccacaactaacaatgccttgttgtattcctgcctttgatgtggatgaaaaaaagggaaaacaaatatttcacttaaactttgtcacttctgctgtacagatatcgagagtttctatggattcacttctatttatttattattattactgttcttattgtttttggatggcttaagcctgtgtataaaaaagaaaacttgtgttcaatctgtgaagcctttatctatgggagattaaaaccagagagaaagaagatttattatgaaccgcaatatgggaggaacaaagacaaccactgggatcagctggtgtcagtccctacttaggaaatactcagcaactgttagctgggaagaatgtattcggcaccttcccctgaggacctttctgaggagtaaaaagactactggcctctgtgccatggatgattcttttcccatcaccagaaatgatagcgtgcagtagagagcaaagatggcttccgtgagacacaagatggcgcatagtgtgctcggacacagttttgtcttcgtaggttgtgatgatagcactggtttgtttctcaagcgctatccacagaacctttgtcaacttcagttgaaaagaggtggattcatgtccagagctcatttcggggtcaggtgggaaagccaagaacttggaaaagataagacaagctataaattcggaggcaagtttcttttacaatgaacttttcagatctcacttccctccgacccctaacttccatgcccacccgtccttttaactgtgcaagcaaaattgtgcatggtcttcgtcgattaataccttgtgtgcagacactactgctccagacgtcgtttccctgataggtagagcagatccataaaaaggtatgacttatacaattaggggaagctaatggagtttattagctgagtatcaatgtctctgcgttgtacggtggtgatgggttttaatgaatatggaccctgaagcctggaaatcctcatccacgtcgaacccacaggactgtgggaagggcagaatcaatccctaagggaaaggaaacctcaccctgagggcatcacatgcactcatgttcagtgtacacaggtcaagtcccttgctctgggctctagttgggagagtggtttcattccaagtgtactccattgtcagtatgctgtttttgtttccttcactccattcaaaaagtcaaaatacaaaatttggcacagcatgccaacgggaggctgtgcccagaccaagcactggaagtgtgcttctaggcatagtcattggttttgcaaaaagagggctcaaatttaaatagaaatttacagctatttgaatggtcagatataccaagaaagaaaaatatttctgttcctcaagaaaacttgctaccctctgtgaggggaattttgctaaacttgacatctttataacatgagccagattgaaagggagtgattttcattcatcttaggtcatgttatttcatatttgtttctgaaggtgcgatagctctgttttaggttttgcttgcgcctgttaattactggaacaccttatttttcattaaaggctttgaaagccaattctcaaaaattcaaaagtgcaaattaacagaacaaaaggaaatccagtagcaactgcagtcaagcgagggagttgacaagataaaccttacgtccattcaagttatatgctggcctatgagagatgagagttgggtcgtttgttctctttgttgatgattttaaaaaaaccctctagaatacacataataacataatgaaagccatatctccatgatatatatgtgcacatatatatacatacatgtgcatgtatgtatcatattaaggacccatggtactcttaaaacactgtagaactctgtgacgcagtaaggaaggggcagatttgtacaaaaacttttctagattccatcagcaaaaaccaacacaggtttgtcacgctgcatgtctggccagctaatctcgggggaaaagctacaagttatttattttattttaagagaataaagtaggtaataatttaagggatcaaattcaaggaggaatgtgcaattttagagcaaagatttgtttaaggcaaatgagactttgggagcatcccattccagttttgtcttttttttctctgaaagaaaaaagcaaaaaataaaataaaattccacttataccttctgacaagtccctaaaggtcttgaaataaaaggttctatgcaagtgcaaagttttatagttatttttattgctgattattactattactatctctgttgtcttaagagtatgtgctgatttcagagacatctcaaattgaaagaatatcagattgcttttaaagtagctgaacgagccacagaatatctgaaattattcattgttgttcctccaccaccccctttctcatggtctgatttttagaagagtggcatcctcgttctaaaatgtaatgatcaccaaatacggccttccatcaaatttgtgaaaactacaacagtataacagtgacaaacctaattctctagcccaaacctggtctgacaatcatttccatttagaagtcattgaatagttttccaaacactttccatgtgtgttagcaaattattcctattttgtgtagatgaggacgttgagactcagagacattcagaggcacgctagaggtctccagcctagcttccagcaccattgggactgaatccaagtactctcactctgaacttcgtggttctgtccactagagactctaatatgcaaacaagcagttcaggaaagaaagcatgctaacacattcatgaagcagtatatgaagttagaagaacaaaaggaaatacaggagatgacaagcaactgagatattgtgatatataatcatgctcttagcttcagctaaattcagctaaattcttgtacactgaaccaatgtcataatcaggcttatttagaaaacactttgaactatgctataaaagattatatcagaattcatattatacatgtgttcacatcagcgctacctgtgatgttttcatgtatttatgtatgtgttataaatacttgatttatacatatacaaatgcacatacgtagtgtgtttgtgtgtttatgtataaatttataggcacacaataatagaggtaattataagtaggatgcggtatgaataatttgcttaaaatatgctaaataaccaaaactgtttaacgtcatgttgctgttagtgcttccatactccacgtgggtaggactacatcacacttttcaactctgtgcagtactgcatgggtggaagacatatttaagataatgtgcttcccaaaacaactgaataaaagccatcccactacattgagtgctttctctggctccttgcaaagaaagatactttttgtaatggtccaggaaaggaacattgctttctttttgtctttcagcacatttgtattatgctcaccttgtctctgtctcactgtgacccctttacacttgagttcagagttcaagcattccaaatataaattggaatgttggcagcccagtggcttgaaggccaatgatgagcagtccaagaccccacagcgagatgagcaactcttaggaattcccacatcctagagtgaatgcaccaactaacagtatagaatgctgtccttttcaaagcgtcctaacagcaggattacctggtcaagtatggactttctttgaatctttcttttcacaaattggactgcctgtaataccaataacattgttgtatctaactaaataaatgactgcatatacacacataccctcaattctcttgcttccccattttctttttcatcccctgtctcaggacttttattttcaatgttgacctttggtttggccatatatcactgttataggaaatctcatgagaggaatggctagtgacccaactctccaaatgtctaagttagtagttacagctgattttttatgatgcataattggaatgtggagcctctgaggttgtgatagcttgtacatgaatttcaaatgtcattctaaagaatgaggggtgggagggatttatagttagaaacgacagtgcaggaaggggtattttcttgttgtcagggctggaatgaatcactgctgctcaagtcaaaggttcttgaatatccttagtttttgcatttcccctccttttcctttgacctttatttatttaattatgtatttatttatttatttatatacttttgctccattcagcacaaacacaaagcaaagcaaaaaaaaaaatatatatatatatatctgtatatgtgttgtaggcaaaacactgtgaatttcacaacaaccaccaccaagcaactattttgccatcttaacatacatctcaggagacgaaatgagaaaagatggggatgtcattttttagtctatgcgtttgaggccaggtccatgtttatttatttctttagtctatgcattaatgaaaatgatcctgagtggaggttagctgaacgttcaatgtactggagcaagcatcataaaagctgctagtagccatgtgtttgaacaggaaaaatattacagaaaatgaaatgtaaaggcctatatcttgcagcttgtatatcttactattgcttaaaaaatgtataaagcagctggaaatgttttaaatacaaggtctttgaattaaatgtggattttaaatatgtaatcccttgacaaatgaccaaattatggtgaactattgctccctgcgttctttgatcattacctatgacttacaaatctgcctggagatgtggacattctgcatttgcttctgtatctggagagatgtttgtatatatccaggccgtatacacacacatttccatatctctctacagatatatttccccttcaatcgtgacctggtatttggaactctccttttcatttggcttatcttccttttaatgtgatgtctctgtgctaatacttaccagttcttgttttgcaatctgttttgaggtccattgctttactaagacccactgcatcttggctgatttcaaagtgacacctgaatacagtgtttaaaaaaaaaaaagttttgtttgtaaatcatgtgaccagcttctctcaacctgacatggaaagtctcttgtactacagtgtatttaataaaaatgatgtcttacaataaataacatactccaaaagagagactaaaaatgaaaaaaaaaaaaaaaaaaaaaaaaa

SEQ ID NO: 9 - protein, 838 aa, Artificial Sequence
  Synthetic
MSSWIRWHGPAMARLWGFCWLVVGFWRAAFACPTSCKCSASRIWCSDPSPGIVAFPRLEPNSVDPENITEIFIANQKRLEIINEDDVEAYVGLRNLTIVDSGLKFVAHKAFLKNSNLQHINFTRNKLTSLSRKHFRHLDLSELILVGNPFTCSCDIMWIKTLQEAKSSPDTQDLYCLNESSKNIPLANLQIPNCGLPSANLAAPNLTVEEGKSITLSCSVAGDPVPNMYWDVGNLVSKHMNETSHTQGSLRITNISSDDSGKQISCVAENLVGEDQDSVNLTVHFAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREHLSVYAVVVIASVVGFCLLVMLFLLKLARHSKFGMKDFSWFGFGKVKSRQGVGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 10 - DNA, 810 bp, Artificial Sequence
  Synthetic
attgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctcctt

SEQ ID NO: 11 - protein, 270 aa, Artificial Sequence
  Synthetic
IVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLL

SEQ ID NO: 12 - DNA, 1437 bp, Artificial Sequence
  Synthetic
aaattatcctgatgtaatttatgaagattatggaactgcagcgaatgacatcggggacaccacgaacagaagtaatgaaatcccttccacagacgtcactgataaaaccggtcgggaacatctctcggtctatgctgtggtggtgattgcgtctgtggtgggattttgccttttggtaatgctgtttctgcttaagttggcaagacactccaagtttggcatgaaagatttctcatggtttggatttgggaaagtaaaatcaagacaaggtgttggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctagggcccttttccccagaccgatccttcccaacgtactcctcagacgggctgagaggatgaacatcttttaactgccgctggaggccacca

SEQ ID NO: 13 - DNA, 890 bp, Artificial Sequence
  Synthetic
gctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggaca

SEQ ID NO: 14 - DNA, 1508 bp, Artificial Sequence
  Synthetic
tcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggctagggcccttttccccagaccgatccttcccaacgtactcctcagacgggctgagaggatgaacatcttttaactgccgctggaggccaccaagctgctctccttcactctgacagtattaacatcaaagactccgagaagctctcgagggaagcagtgtgtacttcttcatccatagacacagtattgacttctttttggcattatctctttctctctttccatctcccttggttgttcctttttctttttttaaattttctttttcttttttttttcgtcttccctgcttcacgattcttaccctttcttttgaatcaatctggcttctgcattactattaactctgcatagacaaaggccttaacaacgtaatttgttatatcagcagacactccagtttgcccaccacaactaacaatgccttgttgtattcctgcctttgatgtggatgaaaaaaagggaaaacaaatatttcacttaaactttgtcacttctgctgtacagatatcgagagtttctatggattcacttctatttatttattattattactgttcttattgtttttggatggcttaagcctgtgtataaaaaagaaaacttgtgttcaatctgtgaagcctttatctatgggagattaaaaccagagagaaagaagatttattatgaaccgcaatatgggaggaacaaagacaaccactgggatcagctggtgtcagtccctacttaggaaatactcagcaactgttagctgggaagaatgtattcggcaccttcccctgaggacctttctgaggagtaaaaagactactggcctctgtgccatggatgattcttttcccatcaccagaaatgatagcgtgcagtagagagcaaagatggcttccgtgagacacaagatggcgcatagtg

SEQ ID NO: 15 - DNA, 3004 bp, Artificial Sequence
  Synthetic
acatttctgcagccgcgcggcgagccattcgcggcggctgctgcagctcctactgcatcttccttctcttcctttcctcgggctccggtctcggagtcggagagcgcgcctcgcttccagagcccccggacccggcgagtcagcgatcgccgagccggccaccatgcccggcagaccgcgccactaggcgctcctcgcggctcccacccggcggcggcggcggcggcggcggcgtccgcgatggtttcagacgctgaaggattttgcatctgatcgctcggcgtttcaaagaagcagcgatcggagatggatgtctctctttgcccagccaagtgtagtttctggcggattttcttgctgggaagcgtctggctggactatgtgggctccgtgctggcttgccctgcaaattgtgtctgcagcaagactgagatcaattgccggcggccggacgatgggaacctcttccccctcctggaagggcaggattcagggaacagcaatgggaacgccagtatcaacatcacggacatctcaaggaatatcacttccatacacatagagaactggcgcagtcttcacacgctcaacgccgtggacatggagctctacaccggacttcaaaagctgaccatcaagaactcaggacttcggagcattcagcccagagcctttgccaagaacccccatttgcgttatataaacctgtcaagtaaccggctcaccacactctcgtggcagctcttccagacgctgagtcttcgggaattgcagttggagcagaactttttcaactgcagctgtgacatccgctggatgcagctctggcaggagcagggggaggccaagctcaacagccagaacctctactgcatcaacgctgatggctcccagcttcctctcttccgcatgaacatcagtcagtgtgaccttcctgagatcagcgtgagccacgtcaacctgaccgtacgagagggtgacaatgctgttatcacttgcaatggctctggatcaccccttcctgatgtggactggatagtcactgggctgcagtccatcaacactcaccagaccaatctgaactggaccaatgttcatgccatcaacttgacgctggtgaatgtgacgagtgaggacaatggcttcaccctgacgtgcattgcagagaacgtggtgggcatgagcaatgccagtgttgccctcactgtctactatcccccacgtgtggtgagcctggaggagcctgagctgcgcctggagcactgcatcgagtttgtggtgcgtggcaaccccccaccaacgctgcactggctgcacaatgggcagcctctgcgggagtccaagatcatccatgtggaatactaccaagagggagagatttccgagggctgcctgctcttcaacaagcccacccactacaacaatggcaactataccctcattgccaaaaacccactgggcacagccaaccagaccatcaatggccacttcctcaaggagccctttccagagagcacggataactttatcttgtttgacgaagtgagtcccacacctcctatcactgtgacccacaaaccagaagaagacacttttggggtatccatagcagttggacttgctgcttttgcctgtgtcctgttggtggttctcttcgtcatgatcaacaaatatggtcgacggtccaaatttggaatgaagggtcccgtggctgtcatcagtggtgaggaggactcagccagcccactgcaccacatcaaccacggcatcaccacgccctcgtcactggatgccgggcccgacactgtggtcattggcatgactcgcatccctgtcattgagaacccccagtacttccgtcagggacacaactgccacaagccggacacgtatgtgcagcacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacaggctctttaatccatctggaaatgatttttgtatatggtgtgaggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctccatgctttggggaaggccaccccaatctacctggacattcttggctagtggtggctggtggtcatgaattcatactctgttgcctcctctctccctgcctcacatctcccttccacctcacaactccttccatccttgactgaagcgaacatcttcatataaactcaagtgcctgctacacatacaacactgaaaaaaggaaaaaaaaagaaagaaaaaaaaaccc

SEQ ID NO: 16 - protein, 839 aa, Artificial Sequence
  Synthetic
MDVSLCPAKCSFWRIFLLGSVWLDYVGSVLACPANCVCSKTEINCRRPDDGNLFPLLEGQDSGNSNGNASINITDISRNITSIHIENWRSLHTLNAVDMELYTGLQKLTIKNSGLRSIQPRAFAKNPHLRYINLSSNRLTTLSWQLFQTLSLRELQLEQNFFNCSCDIRWMQLWQEQGEAKLNSQNLYCINADGSQLPLFRMNISQCDLPEISVSHVNLTVREGDNAVITCNGSGSPLPDVDWIVTGLQSINTHQTNLNWTNVHAINLTLVNVTSEDNGFTLTCIAENVVGMSNASVALTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEEDTFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 17 - DNA, 861 bp, Artificial Sequence
  Synthetic
atcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacaggctctttaatccatctggaaatgatttttgtatatggtgtgaggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctc

SEQ ID NO: 18 - protein, 287 aa, Artificial Sequence
  Synthetic
IVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKIL

SEQ ID NO: 19 - DNA, 1447 bp, Artificial Sequence
  Synthetic
gagtccaagatcatccatgtggaatactaccaagagggagagatttccgagggctgcctgctcttcaacaagcccacccactacaacaatggcaactataccctcattgccaaaaacccactgggcacagccaaccagaccatcaatggccacttcctcaaggagccctttccagagagcacggataactttatcttgtttgacgaagtgagtcccacacctcctatcactgtgacccacaaaccagaagaagacacttttggggtatccatagcagttggacttgctgcttttgcctgtgtcctgttggtggttctcttcgtcatgatcaacaaatatggtcgacggtccaaatttggaatgaagggtcccgtggctgtcatcagtggtgaggaggactcagccagcccactgcaccacatcaaccacggcatcaccacgccctcgtcactggatgccgggcccgacactgtggtcattggcatgactcgcatccctgtcattgagaacccccagtacttccgtcagggacacaactgccacaagccggacacgtatgtgcagcacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacaggctctttaatccatctggaaatgatttttgtatatggtgtgaggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctccatg

SEQ ID NO: 20 - DNA, 1514 bp, Artificial Sequence
  Synthetic
gagtccaagatcatccatgtggaatactaccaagagggagagatttccgagggctgcctgctcttcaacaagcccacccactacaacaatggcaactataccctcattgccaaaaacccactgggcacagccaaccagaccatcaatggccacttcctcaaggagccctttccagagagcacggataactttatcttgtttgacgaagtgagtcccacacctcctatcactgtgacccacaaaccagaagaagacacttttggggtatccatagcagttggacttgctgcttttgcctgtgtcctgttggtggttctcttcgtcatgatcaacaaatatggtcgacggtccaaatttggaatgaagggtcccgtggctgtcatcagtggtgaggaggactcagccagcccactgcaccacatcaaccacggcatcaccacgccctcgtcactggatgccgggcccgacactgtggtcattggcatgactcgcatccctgtcattgagaacccccagtacttccgtcagggacacaactgccacaagccggacacgtatgtgcagcacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacaggctctttaatccatctggaaatgatttttgtatatggtgtgaggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctccatgctttggggaaggccaccccaatctacctggacattcttggctagtggtggctggtggtcatgaattc

SEQ ID NO: 21 - DNA, 950 bp, Artificial Sequence
  Synthetic
cacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacaggctctttaatccatctggaaatgatttttgtatatggtgtgaggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctccatgctttggggaaggccaccccaatctacctggacattcttggctagtggtggctggtggtcatgaattc